(19) United States
(12) Patent Application Publication
(10) Pub. No.: US 2023/0348572 A1
(43) Pub. Date: Nov. 2, 2023

(54) SINGLE DOMAIN ANTIBODIES BINDING TO SARS-COV-2 SPIKE PROTEIN

(71) Applicant: THE ROSALIND FRANKLIN INSTITUTE, DIDCOT, OXFORDSHIRE (GB)

(72) Inventors: Jiandong HUO, Oxfordshire (GB); Raymond OWENS, Oxfordshire (GB); James NAISMITH, Oxfordshire (GB); David STUART, Oxfordshire (GB)

(21) Appl. No.: 17/923,142

(22) PCT Filed: May 4, 2021
(86) PCT No.: PCT/GB2021/051069

§ 371 Date: Nov. 3, 2022

(30) Foreign Application Priority Data
May 4, 2020 (GB) 2006589.2
Jul. 14, 2020 (GB) 2010861.9

(51) Int. Cl. C07K 16/10; A61P 31/14; G01N 33/569

(52) U.S. Cl. CPC C07K 16/1003; A61P 31/14; G01N 33/56983; C07K 2317/569; C07K 2317/76; C07K 2317/92; C07K 2317/565; C07K 2317/567; G01N 2333/165; G01N 2469/10; C07K 2317/35; C07K 2317/33

(57) ABSTRACT

The present invention relates to improved single domain antibodies that target SARS-CoV-2, the use of said single domain antibodies in treating and/or preventing coronavirus, as well as the use of said single domain antibodies in the detection and diagnosis of coronavirus using various methods, assays and kits.

### FIELD OF THE INVENTION

The invention provides improved single domain antibodies that target SARS-CoV-2, the use of said single domain antibodies in treating and/or preventing coronavirus, as well as the use of said single domain antibodies in the detection and diagnosis of coronavirus using various methods, assays and kits.

### BACKGROUND OF THE INVENTION

Single domain antibodies or nanobodies are recombinant antigen-specific variable domains (VHHs) derived from the heavy chain only subset of camelid immunoglobulins. Their small molecular size, facile expression, high affinity and stability have combined to make them unique targeting reagents with numerous applications in the biomedical sciences. Nanobodies have emerged as alternatives to conventional antibodies for the development of diagnostic reagents, and for use in non-invasive bioimaging. The therapeutic potential of nanobodies is being explored in a number of indications, including oncology and inflammatory diseases (Revets, De Baetselier et al. 2005, Chanier and Chames 2019).

The very recent emergence of the SARS-nCoronavirus 2 (SARS-Cov-2) (Zhou, Yang et al. 2020) has focused attention on the use of nanobodies in virology for research into the basic biology of the virus but also as possible anti-viral agents. Clearly, an understanding of the structure-function relationships of the key protein of SARS-CoV2 is required to develop vaccines and therapeutics. In common with other coronaviruses, the single positive strand RNA genome of SARS-Cov2 encodes four major structural proteins, Spike (S), Envelope (E), Membrane (M) and Nucleocapsid (NP). Of these, the S protein plays the key role in viral attachment, fusion and entry into host cells and comprises a N-terminal (S1) subunit responsible for virus-receptor binding and C-terminal domain (S2) that mediates membrane fusion (Li 2016). The cell surface receptor for SARS-CoV2, like the related coronavirus SARS-CoV, is angiotensin-converting enzyme 2 (ACE-2) and the interaction has been mapped to an approximately 300 amino acid receptor-binding domain (RBD) in S1 (Wan, Shang et al. 2020, Yan, Zhang et al. 2020).

We report the surprising identification of a number of nanobodies with high affinity for the RBD by screening a naïve llama VHH library in vitro by phage display technology. We have analysed the binding properties of the VHH with the highest affinity (Kd=5.2 nM) and that it blocks the interaction between ACE-2 and the RBD domain of SARS-CoV-2.

There is currently no treatment for COVID-19, the disease caused by SARS-CoV-2, and therefore a critical need for effective treatments exists. Further, suitable tools for the rapid and efficient detection of SARS-CoV-2 are required to enable accurate diagnosis and monitoring of the virus.

### SUMMARY OF THE INVENTION

The present invention provides single domain antibodies that specifically bind to the receptor biding domain of the S-protein of SARS-CoV-2.

In a first aspect, a single domain antibody comprising a complementary determining region, complementary determining region 3 (CDR3), is provided.

In a second aspect, a single domain antibody comprising a CDR2 and a CDR3 is provided.

In a third aspect, a single domain antibody comprising a CDR1, a CDR2 and a CDR3 is provided.

In a further aspect, an anti-SARS-CoV-2 single domain antibody comprising an amino acid sequence having at least 70% identity to a sequence selected from the group consisting of: SEQ 61, 62, 63, 64, 65, 66, 67, 68, 69, 70, 71, 72, 73, 87, 88, 89, 90, 91, 92, 93, 94, 95, 96, 97, 98, 99, 101, 102, 103, 104, 105, 106 and 107 is provided.

In a further aspect, a polynucleotide sequence is provided encoding a single domain antibody of the invention.

In a further aspect, an affinity matured mutant of a single domain antibody of the invention is provided.

In a further aspect, a method for producing a single domain antibody of the invention is provided.

In a further aspect, a pharmaceutical composition comprising a single domain antibody of the invention is provided.

In a further aspect, a single domain antibody of the invention or a pharmaceutical composition of the invention for use in medicine is provided.

In a further aspect, a method for the treatment of a coronavirus in a subject is provided, comprising administering to a subject a therapeutically active amount of a single domain antibody of the invention.

In a further aspect, the use of a single domain antibody of the invention in the manufacture of a medicament for use in the treatment and/or prevention of a coronavirus is provided.

In a further aspect, methods for the detection of a coronavirus protein are provided.

In a further aspect, methods for diagnosing a coronavirus infection in a subject are provided.

### DESCRIPTION OF FIGURES

FIG. 1 The Spike protein of SARS-CoV-2 drives infection.

(a) Schematic representation of the Spike protein of SARS-CoV-2. The Spike protein is composed of the S1 and S2 subunits. The S1 domain contains the receptor binding domain (RBD) which is highlighted in red. Using the RBD, the trimeric Spike molecule binds to ACE2 on human cells.

(b) Selected residues of the RBD which are shared between SARS-CoV-2 and SARS-CoV-1 are highlighted in grey (SARS-CoV-2) and shown in light grey text (SARS-CoV-1). Residues which contact ACE2 are highlighted in bold for each sequence. Other sequence differences are in black text, with conservative substitutions indicated by colons (:) below. Residues in contact with H11-H4 nanobody are boxed.

(c) Camelids have antibodies that are dimers of a single chain. The constant region is in black and the variable region is in grey. When the VHH domain is expressed on its own, it is termed a nanobody. A topology diagram shows the nanobody is composed of two b-sheets. Three loops—complementary-determining region 1 (CDR1), CDR2 and CDR3—control antigen binding and are highlighted in darker grey.

FIG. 2 Laboratory-matured nanobodies bind to RBD and spike proteins with high affinity.

a) Maturation by mutagenesis of CDR3 region of H11 resulted in H11-D4 and H11-H4. The five changes from the parent are shown in bold.

(b) SPR sensorgram showing that H11-H4 bound to RBD (immobilized as RBD-Fc on the chip) with 5 nM affinity. A repeat experiment is shown in FIG. 6C and H11-D4 data are provided in FIG. 6D.

(c) RBD was bound by ACE2 (immobilized as ACE2-Fc on the chip). When RBD was pre-mixed with H11-H4, there was no binding, indicating that H11-H4 and ACE2 compete for binding to RBD. Similar results were observed using spike protein instead of RBD. The antibody E08R (anti-Caspr2 Fab) was used as a negative control. Data for H11-D4 are provided in FIG. 6E.

(d) RBD was bound by CR3022 (immobilized as CR3022-Fc on the chip). When RBD was pre-mixed with H11-H4, binding occurred with similar on and off rates, indicating that H11-H4 and CR3022 recognize different epitopes on RBD. The response for the RBD H11-H4 mixture was larger, consistent with an H11-H4-RBD complex binding to CR3022. Antibody E08R was again used as a negative control. The spike protein shows binding to CR3022 in the presence or absence of H11-H4. Data for H11-D4 are provided in FIG. 6F.

(e) ITC measurements show a KD of 12±1.5 nM and a 1:1 ratio for H11-H4 and RBD association. Replicates and data for H11-D4 are provided in FIG. 7A.

(f) ITC measurements show a KD of 44±3 nM and a 1:1 ratio for association between spike protein and H11-H4. Replicates and data for H11-D4 are provided in FIG. 7B.

FIG. 3 In vitro biological activity of H11-H4.

(a) Schematic representation of H11-H4-Fc, the IgG1 Fc fusion used in vitro assays.

(b) Biotinylated RBD was mixed with analytes at various ratios and then added to MDCKSIAT1 cells stably expressing human ACE2. The amount of biotinylated RBD bound was measured. Experiments were performed in duplicate with the mean±SD are shown.

(c) Biotinylated ACE2-Fc was mixed with analytes at various ratios and then added to MDCKSIAT1 cells stably expressing RBD. The amount of biotinylated ACE2-Fc bound was measured. Experiments were performed in duplicate with the mean±SD are shown.

(d) H11-H4-Fc (6 nM, 95% Cl 3-9 nM) and H11-D4-Fc (18 nM, 95% Cl 9-68 nM) show potent neutralization of live wild type virus. 95% confidence intervals are shown as dashed lines. Raw data plots are shown in FIG. 8.

(e) H11-H4-Fc shows similar neutralization (ND50 4 nM) of live wild type virus in a Vero cellbased assay in Oxford. CR3022 is shown as a positive control for this assay system and is similar to a previous report. Data are presented as mean and s.d. of n=2 technical replicates. Experimental plates are shown in FIG. 8.

FIG. 4 Structural biology of H11-H4 bound to Spike and RBD.

(a) EM structure of Spike (S1) trimer with each of three chains bound to one H11-H4 nanobody. The ‘up’ configured monomer of the Spike is colored light pink with its RBD highlighted in red. The other monomers are colored pale cyan and wheat throughout. Each monomer of Spike has bound one H11-H4 nanobody, the three H11-H4 nanobodies are colored yellow.

(b) Crystal structures of the H11-H4-RBD and H11-D4-RBD complexes were superimposed via the RBD (red), showing that both nanobodies recognize the same RBD epitope. H11-D4 is colored orange and H11-H4 yellow.

(c) There is 7° pivot between H11-H4 and H11-D4 which means the nanobodies are shifted.

(d) The loops CDR1, CDR2 and CDR3 of H11-H4 control recognition and are highlighted in magenta.

(e) A 90° rotation of the structure shown in FIG. 4d, showing the residues in contact with RBD. H11-H4 residues are labelled in yellow with carbon atoms colored yellow, nitrogen atoms blue and oxygen atoms red. RBD is shown as a white surface with contact points (<4.0Å) highlighted in dark grey.

(f) The same view as in FIG. 4c, showing the RBD residues in contact with H11-H4 (which has been omitted). Selected RBD residues are labelled, with carbons colored in grey, other atoms are colored as FIG. 4c.

(g) LigPlot detailing the interaction between H11-H4 (residues shown in grey, top) and RBD (residues shown in black, below). The hydrogen bonds are shown in black dashes and van der Waals interactions in light grey dashes.

(h) Arg52 of CDR2 of H11-H4 stacks against Phe490 of RBD and makes salt contacts with Glu484. In addition, it makes hydrogen bonds to the main chain Ser103 (side chain omitted in Figure) and Tyr109.

FIG. 5 H11-H4 and CR3022 have different binding epitopes on RBD and show additive neutralization activities.

(a) Superposition using the RBD domains of the H11-H4-RBD complex (colored as FIG. 4e) with the RBD—ACE2 complex10 (ACE2 colored in pale blue). When H11-H4 is bound to RBD it would prevent ACE2 binding due to steric clashes.

(b) The region of RBD that engages ACE2 only has a small overlap with that region recognized by H11-H4. The RBD is shown as a molecular surface, those regions that only contact ACE2 are highlighted in dark blue, whilst those that only contact H11-H4 are in red. The two helices and turn of ACE2 that contact RBD are shown in cartoon and colored as FIG. 5a. CDR1, 2 and 3 of H11-H4 are labelled, shown in cartoon and colored in yellow. Tyr489 and Gln493, that contribute significantly to both binding sites are highlighted in pale green.

(c) The ternary complex H11-D4-RBD—CR3022 shows the nanobody and antibody recognize entirely different epitopes. CR3022 is colored in pale pink and salmon, the other molecules as FIG. 4b.

(d) Neutralization assay for H11-H4-Fc in the presence of CR3022 at a fixed concentration of 84 nM. The solid gray line represents the control values, with no neutralizing agent. Dashed lines are 50% and 10% of the control, respectively. Data are presented as mean and s.d. of n=2 technical replicates. The shift in the H11-H4-Fc neutralization curve and the measured ND50 of 2 nM indicate additivity. The experimental plates are shown in FIG. 9D.

FIG. 6 Biophysics of the nanobody binding to RBD.

(a) Raw sensorgrams for the H11 parent nanobody identified from a naïve library screen.

(b) Calculation of the KD of the H11 parent nanobody.

(c) A repeat of FIG. 2B, the sensorgram for H11-H4 binding to RBD-Fc.

(d) The sensorgrams for H11-H4 binding to RBD-Fc.

(e) Binding of analytes to ACE2-Fc immobilized on the chip. H11-D4 behaved identically to H11-H4 (FIG. 2C).

(f) Binding of analytes to CR2022-Fc immobilized on the chip. H11-D4 behaved identically to H11-H4 (FIG. 2D).

FIG. 7 ITC measurements of nanobodies binding to RBD or Spike.

(a) Three independent ITC measurement of H11-H4 and H11-D4 binding to RBD. The errors are s.e.m. for three independent experiments, including the one shown in FIG. 2D.

(b) As above, but with the Spike protein.

FIG. 8 Neutralization of live virus at Public Health England.

The percentage reduction in plaques arising from virus is plotted against increasing (left to right) concentration of

(a) H11-H4-Fc (6 nM, 95% Cl 3-9 nM)

(b) H11-D4-Fc (18 nM, 95% Cl 9-68 nM). The confidence intervals are shown as dashed lines. In FIG. 3d,e 5d % infectivity (% infectivity=100−% plaque reduction) is plotted against decreasing (left to right) concentration of the agent.

(c) The experimental plate with the codes noted below. The plaques caused by the virus are visible.

FIG. 9 Neutralization of live virus at Oxford University.

The concentration of neutralizing agent was held constant across a row and decreased on subsequent rows. The agent was tested against high and low virus concentrations.

(a) Control plate no agent.

(b) CR3022.

(c) H11-H4-Fc.

(d) H11-H4-Fc varied and CR3022 held constant at 84 nM.

Images on left show plaques before pen counting, on the right the pen counts are shown.

FIG. 10 Cryo-EM of the H11-H4-Spike complex.

(a) Unbiased 2D class averages of the H11-H4-Spike (from SARS-CoV-2) complex

(b) 2D class averages selected for further processing.

(c) Estimated resolution using FSC criteria.

(d) Particle orientation distribution for the final map, showed no preferred orientation.

(e) Final cryo-EM map (shaded according to local resolution).

(f) Ribbon diagram of the complex (cryo-EM map shown as grey volume contoured at 4 s in chimera).

(g, h, i) Cryo-EM density with ribbon for each of the three H11-H4 nanobodies bound RBD, contoured at 4 s in chimera. The cryo-EM map used in panels f-i had its amplitudes scaled based on the refined coordinates using LocScale.

FIG. 11 Cryo-EM of the H11-D4—Spike complex.

(a) Unbiased 2D class averages of the H11-D4-Spike (from SARS-CoV-2) complex.

(b) 2D class averages selected for further processing.

(c) Estimated resolution using FSC criteria.

(d) Particle orientation distribution for the final map, showed no preferred orientation.

(e) Final cryo-EM map (shaded according to local resolution).

(f) Ribbon diagram of the complex (cryo-EM map shown as grey volume contoured at 3.8 s in chimera).

(g, h, i) Cryo-EM density with ribbon for each of the three H11-H4 nanobodies bound RBD, contoured at 3.8 s in chimera. The cryo-EM map used in panels f-l had its amplitudes scaled based on the refined coordinates using LocScale.

FIG. 12 Further analysis of the cryo-EM nanobody-Spike complexes.

(a) The H11-D4—Spike complex is colored as FIG. 4a.

(b) The H11-H4—Spike shown in surface and colored in FIG. 4a, has revealed there is an interaction between H11-H4 bound to a down subunit and the up RBD.

(c) A close of up of the interaction shown in FIG. 9a. Shown in dark blue is the down RBD from the Spike structure (6vyb13. The down RBD has shifted by around 2Å.

(d) A model constructed from the closed structure of the Spike bound to H11-H4 reveals no clash, indicating the nanobody will recognize this form too. The model was constructed by superimposing the H11-H4 RBD complex onto the EM structure of the closed form of Spike (6vxx13).

FIG. 13 Further structural analysis of nanobody-RBD crystal structures.

(a) Superimposing the RBDs from the complex with ACE2 (colored light grey PDB 6m0j) and the complex with H11-H4. By using residues 484 to 510 indicated a hinging movement occurs within the RBD. As a result of this hinge, the Cα of His 519 has shifted 2.1Å.

(b) The binding of H11-H4 results in local shifts at Val483 of the RBD from the ACE2 complex10,11.

(c) 2Fo-Fc electron density map contoured at 2 s for residues at the H11-H4-RBD interface.

(d) 2Fo-Fc electron density map contoured at 2 s for residues at the H11-D4-RBD interface.

FIG. 14 H11-H4 and VHH72 recognize different epitopes.

(a) VHH72 (black) and H11-H4 (yellow) recognize different epitopes on RBD (red).

(b) The epitopes for VHH72 and CR3022 (pale pink and lilac) overlap. The figure was generated by superimposing RBD from the CR3002 RBD complex, VHH7 MERS RBD complex and the H11-H4-RBD complex.

(c) Analysis of the crystal packing in the H11-H4-RBD complex revealed that the crystal contact uses the same epitope on the RBD. The crystal contact surface used by H11-H4 is different from that used by VHH72 (the nanobodies are rotated by 180° around vertical axis that passes through the center of nanobody).

(d) Close up of the crystal contact revealed an antiparallel b-sheet type interaction.

FIG. 14 H11-H4 and VHH72 recognize different epitopes.

(a) VHH72 (black) and H11-H4 (yellow) recognize different epitopes on RBD (red).

(b) The epitopes for VHH72 and CR3022 (pale pink and lilac) overlap. The figure was generated by superimposing RBD from the CR3002 RBD complex18 (PDB 6YLA), VHH72 SARS-CoV-1 RBD complex25 (PDB 6WAQ) and the H11-H4-RBD complex.

(c) Analysis of the crystal packing in the H11-H4-RBD complex revealed that the crystal contact uses the same epitope on the RBD. The crystal contact surface used by H11-H4 is different from that used by VHH72 (the nanobodies are rotated by 180° around vertical axis that passes through the centre of nanobody).

(d) Close up of the crystal contact revealed an antiparallel β-sheet type interaction.

FIG. 15 Structure of H11-D4 nanobody bound to the RBD domain.

(a) The crystal structure of the H11-D4 and RBD complex. The loops CDR1, CDR2 and CDR3, which control epitope recognition, are highlighted in dark grey.

(b) A 90° rotation of the structure shown in panel a, showing the residues in contact with RBD. RBD is shown as a surface with contact points (4.0 Å)

(c) LigPlot⁴² detailing the interaction between H11-D4 (residues shown in light grey, top) and RBD (residues shown in black, bottom). Arg27, which is not well ordered in the crystal structure, was not included in this analysis. Hydrogen bonds are shown in black dashes and van der Waals interactions in light grey dashes.

(d) The same view as in panel b, showing the RBD residues in contact with H11-D4. Selected RBD residues are labelled in black, with carbons colored in grey.

FIG. 16 The key interactions that stabilize the H11-D4RBD complex.

(a) The aromatic ring of Tyr449 in RBD stacks against a hydrophobic patch on H11-D4 at Asn101.

(b) Arg52 of CDR2 of H11-H4 stacks against Phe490 of RBD and makes salt bridge contacts with Glu484. In addition, it makes hydrogen bonds to Ser103 and Tyr109.

(c) Arg98 is the only change in the CDR3 loop that does not contact RBD; instead, this residue stabilizes the loop structure.

(d) Glu108, Arg103 and Trp113 of H11-D4 play a key role in stabilizing the CDR3 loop.

FIG. 17

Interaction between SARS-2 receptor binding domain and nanobody H11 SDS page of purified RBD (lane 1), purified Nb H11 (lane 2) and RBD-Nb11 co-eluted from size exclusion column showing assembly into a 1:1 complex.

FIG. 18

(a) Competition assay NbRBD_H11-D4 with ACE2-Fc IgG immobilized onto the sensor chip. NbRBD_H11-D4 is competitive with ACE-2 for binding to RBD.

(b) Competition assay NbRBD_H11-D4 with CR3022 IgG immobilized onto the sensor chip. E08R Fab is a negative control. NBRBD_H11-D4 and CR3022 are not competitive for binding to RBD.

FIG. 19

(a) All three RBDs in the Spike S1 trimer bind NBRBD_H11-D4. The ‘up’ configured chain is colored orange with the RBD highlighted in red and the bound nanobody is in yellow. One of the ‘down’ chains of the S1 protein is colored pale blue and the nanobody pale purple; the other ‘down’ chain is colored green with its bound nanobody in brown.

(b) A close up view of the residues that form the NBRBD_H11-D4 RBD interface, selected RBD residues are labelled in black, with carbons colored in gray, oxygen red and nitrogen blue. Selected NBRBD_H11-D4 atoms are labelled in yellow, carbons are colored yellow other atoms colored as RBD.

FIG. 20

Ligplot (Laskowski and Swindells, 2011) detailing the interaction between NBRBD_H11-D4 and RBD.

FIG. 21

The ternary complex CR3022, RBD and NBRBD_H11-D4 shows the nanobody and antibody recognise entirely different epitopes.

FIG. 22

Superposition of the NBRBD_H11-D4 and RBD from the ternary complex as shown in FIG. 21 with the RBD ACE-2 complex {Wan et al., 2020, #55974} shows that when NBRBD_H11-D4 is bound to RBD it would prevent ACE-2 binding to RBD by steric clashes.

### DETAILED DESCRIPTION OF THE INVENTION

Various antibody and single-domain antibodies to SARS-CoV-2 are known, for example those is CN111303279, CN111647076, CN111333722, Wu et al., 2020 (Cell Host & Microbe, vol. 27, pg 891-898, 14.05.2020) and Dong et al., 2020 (Emerging Microbes and Infections vol 9(1), pg 1034-1036, 22.05.2020) Antibodies, including nanobodies, raised to the S protein of SARS-CoV or the related MERS-CoV have been shown to both block virus-receptor binding and in some cases neutralize the virus both in vitro and in vivo (Zhu, Chakraborti et al. 2007, Zhao, He et al. 2018). It has also been shown that antibodies from infected patients bind to the S protein et al. (Jan ter Meulen Edward N. van den Brink Leo L. M. Poon 2006, Prabakaran, Gan et al. 2006, Zhu, Chakraborti et al. 2007). Given the role of the S protein in the pathogenicity of the SARS-CoV2, and potential as a target for vaccine and therapeutic development (Du, He et al. 2009), we have focused on producing single domain antibodies to the RBD of the virus for structural and functional studies.

“Conservative substitution” as used herein refers to amino acid substitutions that do not materially affect the function of a protein (for example the ability to bind to a specific target, in particular the coronavirus spike protein of SARS-CoV-2 in the context of the invention, or the ability to elicit an immune response in a subject). The skilled person readily understands the properties of amino acids and can readily make a conservative substitution without materially altering the properties of the resulting polypeptide. Examples of conservative substitutions are provided in the table below.

| Class | Exchangeable amino acids |
|---|---|
| Aliphatic | Glycine, Alanine, Valine, Leucine, |
| | Isoleucine |
| Hydroxyl or Sulfur/Selenium- | Serine, Cysteine, Threonine, Methionine |
| containing | |
| Aromatic | Phenylalanine, Tyrosine, Tryptophan |
| Basic | Histidine, Lysine, Arginine |
| Acidic and their Amide | Aspartate, Glutamate, Asparagine, |
| | Glutamine |

“Deletion” as used herein refers to the removal of an amino acid in a polypeptide sequence (i.e. the replacement of one amino acid with no amino acid such that the amino acid sequence is one amino acid shorter in length). Deletion can also refer to polynucleotide sequences and the removal of one nucleic acid from a polynucleotide sequence (the replacement of one nucleic acid with no nucleic acid such that the polynucleotide sequence is one nucleic acid shorter in length).

“Identity” as used herein is the degree to which two sequences are related, as determined by comparing two or more polypeptide of polynucleotide sequences. Identity can be determined using the degree of relatedness of two sequences to provide a measurement of to what extent the two sequences match. Numerous programs are well known by the skilled person for comparing polypeptide or polynucleotide sequences, for example (but not limited to the various BLAST and CLUSTAL programs. Percentage identity can be used to quantify sequence identity. To calculate percentage identity, two sequences (polypeptide or nucleotide) are optimally aligned (i.e. positioned such that the two sequences have the highest number of identical residues at each corresponding position and therefore have the highest percentage identity) and the amino acid or nucleic acid residue at each position is compared with the corresponding amino acid or nucleic acid at that position. In some instances, optimal sequence alignment can be achieved by inserting space(s) in a sequence to best fit it to a second sequence. The number of identical amino acid residues or nucleotides provides the percentage identity, i.e. if 9 residues of a 10 residue long sequence are identical between the two sequences being compared then the % identity is 90%. Percentage identity is generally calculated along the full length of the two sequences being compared.

“Insertion” refers the addition of an amino acid in a polypeptide sequence (i.e. insertion of one amino acid means one new amino acid is added into in an existing amino acid sequence such that the amino acid sequence is one amino acid longer in length). Insertion can also refer to polynucleotide sequences and the addition of one nucleic acid to a polynucleotide sequence (i.e. insertion of one nucleic acid means one new nucleic acid is added into in an existing polynucleotide sequence such that the nucleic acid sequence is one amino acid longer in length).

“Modification” as used herein refers to an alteration of an amino acid residue in a polypeptide sequence. The modification can be a substitution, deletion or insertion, as defined herein. Modification can also refer to polynucleotide sequences.

“Single domain antibody” as used herein refers to a variable region of a heavy chain of an antibody, wherein the variable region is derived from a heavy chain only (i.e. devoid of a light chain) subset of camelid immunoglobulins. The term single domain antibody can be used interchangeably with (variable domain of camelid heavy-chain-only antibody, VHH) and Nanobody®. In the context of the invention a single domain antibody is used to refer to a single heavy chain variable region that can bind the spike protein of a coronavirus, preferably SAR-CoV-2. The antibody can be affinity matured, humanized or modified, as described herein. This single domain antibody can be conjugated to other components.

“Substitution” as used herein refers the replacement of amino acid with a different amino acid. Substitution can also refer to polynucleotide sequences, i.e. the replacement of one nucleic acid with a different nucleic acid. A substitution can be a conservative substitution, as defined above.

The single domain antibodies of the invention are based on 13 VHH sequences having positive binding to the receptor binding domain of the S protein of SARS-CoV-2, namely NbRBD_H11, NbRBD_A7, NbRBD_F9, NbRBD_C10, NbRBD_B11, NbRDB_E11, NbRBD_D1, NbRBD_G7, NbRBD_F5, NbRBD_G11, NbRBD_B4, NbRBD_G9 and NbRBD_C7 (amino acid sequences provided as SEQ ID NOs: 61-73, polynucleotide sequences provided as SEQ ID NOs: 74-86 respectively). Affinity matured versions of NbRBD_H11 have also been provided, namely NbRBD_H11-D4, NbRBD_H11-H4, NbRBD_H11-H6, NbRBD_H11-A10, NbRBD_H11-B5, NbRBD_H11-A7, NbRBD_H11-F7, NbRBD_H11-F6, NbRBD_H11-G8, NbRBD_H11-D1, NbRBD_H11-A9, NbRBD_H11-C6, NbRBD_H11-E3, NbRBD_H11-F4, NbRBD_H11-C5, NbRBD_H11-C2, NbRBD_H11-B11, NbRBD_H11-A3, NbRBD_H11-D12, NbRBD_H11-D6 and NbRBD_H11-F8 (amino acid sequences provided as SEQ ID NOs: 87-107, polynucleotide sequences provided as SEQ ID NOs: 108-128 respectively).

Specific amino acid sequences are provided herein to define the amino acid sequences of specified CDRs. For convenience, these are listed in Tables 1 and 2 below. Single domain antibodies of the invention comprising these specified CDR sequences can comprise one or more modifications, as detailed herein, and will retain binding affinity for a coronavirus peptide, preferably the receptor binding domain of the S protein of SARS-CoV-2.

TABLE 1
CDR alignments of primary hits (ELISA A405 > 1.5)

| ID | CDR1 | SEQ ID NO | CDR2 | SEQ ID NO | CDR3 | SEQ ID NO |
|---|---|---|---|---|---|---|
| NbRBD_H11 | GRTFSTAA | 1 | IRWSGGSA | 2 | AQTRVTRSLLSDYATWPYDY | 3 |
| NbRBD_A7 | GFTFDDYA | 4 | ISWNGGGT | 5 | AKDFHRYGSSTLEYDY | 6 |
| NbRBD_F9 | GFTFSSYF | 7 | IGDRGNT | 8 | YSRNRILQHY | 9 |
| NbRBD_C10 | GFAFSSYD | 10 | IDSGGGVT | 11 | NAVDGLGYLEYDY | 12 |
| NbRBD_B11 | GFTFDDYA | 13 | IYSYISTT | 14 | AARRLDTTDYKY | 15 |
| NbRDB_E11 | GFTFSNYA | 16 | IGSDGRHP | 17 | LPGWSTVGTFLDA | 18 |
| NbRBD_D1 | GFTFSSYG | 19 | INSGGGST | 20 | AKDASEDAGRLYWTDY | 21 |
| NbRBD_G7 | GFTFDTYD | 22 | EDSTGNK | 23 | AKGLRSWGA | 24 |
| NbRBD_F5 | GTIFRINA | 25 | ISNGDSGD | 26 | YCNVEASWPHKY | 27 |
| NbRBD_G11 | GFTFDDYG | 28 | VNSGGGT | 29 | AKRDGSWWGYTTDY | 30 |
| NbRBD_B4 | GFTFSSYW | 31 | INTGGDTT | 32 | ARTGVVRGPGDLDA | 33 |
| NbRBD_G9 | GFTFYDYH | 34 | IDTGGGRT | 35 | VRDGDARGPDYDY | 36 |
| NbRBD_C7 | GFTFSSYD | 37 | ISGSDSTT | 38 | ARPLGQYTS | 39 |

TABLE 2
CDR alignments of affinity-matured hits (Competition >/= 50%)

| | | SEQ | | SEQ | | SEQ |
|---|---|---|---|---|---|---|
| Clone ID | CDR1 | ID NO | CDR2 | ID NO | CD3 | ID NO |
| PARENT | GRTESTAA | 1 | IRWSGGSA | 2 | AQTRVTRSLLSDYATWPYDY | 3 |
| NbRBD_H11-D4 | GRTESTAA | 1 | IRWSGGSA | 2 | ARTENVRSLLSDYATWPYDY | 40 |
| NbRBD_H11-H4 | GRTESTAA | 1 | IRWSGGSA | 2 | AQTHYVSYLLSDYATWPYDY | 41 |
| NbRBD_H11-H6 | GRTFSTAA | 1 | IRWSGGSA | 2 | AGSKITRSLLSDYATWPYDY | 42 |
| NbRBD_H11-A10 | GRTENTAA | 1 | IMWSGGSA | 2 | AGFSATRSLLSDYATWPYDY | 43 |
| NbRBD_H11-B5 | GRTESTAA | 1 | IRWSGGSA | 2 | ASYQATRSLLSDYATWPYDY | 44 |
| NbRBD_H11-A7 | GRTESTAA | 1 | IRWSGGSA | 2 | AQTDNVRALLSDYATWPYDY | 45 |
| NbRBD_H11-F7 | GRTESTAA | 1 | IRWSGGSA | 2 | AIFSNVRSLLSDYATWPYDY | 46 |
| NbRBD_H11-F6 | GRTESTAA | 1 | IRWSGGSA | 2 | ARTSNVRSLLSDYATWPYDY | 47 |
| NbRBD_H11-G8 | GRTESTAA | 1 | IRWSGGSA | 2 | AGSGVTRSLLSDYATWPYDY | 48 |
| NbRBD_H11-D1 | GRTFSTAA | 1 | IRWSGGSA | 2 | AGWRATRSLLSDYATWPYDY | 49 |
| NbRBD_H11-A9 | GRTFSTAA | 1 | IRWSGGSA | 2 | APYEATRSLLSDYATWPYDY | 50 |
| NbRBD_H11-C6 | GRTESTAA | 1 | IRWSGGSA | 2 | AQTGYTSWLLSDYATWPYDY | 51 |
| NbRBD_H11-E3 | GRTFSTAA | 1 | IRWSGGSA | 2 | AVYHATRSLLSDYATWPYDY | 52 |
| NbRBD_H11-F4 | GRTESTAA | 1 | IRWSGGSA | 2 | AESTITRSLLSDYATWPYDY | 53 |
| NbRBD_H11-C5 | GRTFSTAA | 1 | IRWSGGSA | 2 | AQTSYVSFLLSDYATWPYDY | 54 |
| NbRBD_H11-C2 | GRTFSTAA | 1 | IRWSGGSA | 2 | AGSRATRSLLSDYATWPYDY | 55 |
| NbRBD_H11-B11 | GRTFSTAA | 1 | IRWSGGSA | 2 | AETLNTRSLLSDYATWPYDY | 56 |
| NbRBD_H11-A3 | GRTFSTAA | 1 | IRWSGGSA | 2 | ARSDNVRSLLSDYATWPYDY | 57 |
| NbRBD_H11-D12 | GRTFSTAA | 1 | IRWSGGSA | 2 | ADWGVTRSLLSDYATWPYDY | 58 |
| NbRBD_H11-D6 | GRTFSTAA | 1 | IRWSGGSA | 2 | ASSSVTRSLLSDYATWPYDY | 59 |
| NbRBD_H11-F8 | GRTESTAA | 1 | IRWSGGSA | 2 | ADASATRSLLSDYATWPYDY | 60 |

In one aspect, a single domain antibody comprising a complementary determining region, complementary determining region 3 (CDR3), is provided. In one embodiment, a single domain antibody comprises a complementary determining region selected from CDR1, complementary determining region 2 (CDR2) or complementary determining region 3 (CDR3) is provided. In one embodiment, a single domain antibody comprises at least one complementary determining region selected from CDR1, CDR2 or CDR3 is provided. In one embodiment, a single domain antibody comprises at least two complementary determining regions selected from CDR1, CDR2 or CDR3. In one embodiment, single domain antibody comprises three complementary determining regions: CDR1, CDR2, and CDR3 is provided.

In one embodiment, a single domain antibody comprising a complementary determining region 3 (CDR3) selected from the group consisting of SEQ ID NOs: 3, 6, 9, 12, 15, 18, 21, 24, 27, 30, 33, 36, 39, 40, 41, 42, 43, 44, 45, 46, 47, 48, 49, 50, 51, 52, 53, 54, 55, 56, 57, 58, 59 and 60 is provided, wherein the amino acid sequences of CDR3 comprise between 0 and 10 amino acid modifications. In one embodiment, an single domain antibody comprising a complementary determining region 3 (CDR3) selected from the group consisting of SEQ ID NOs: 3, 6, 9, 12, 15, 18, 21, 24, 27, 30, 33, 36, 39, 40, 41, 42, 43, 44, 45, 46, 47, 48, 49, 50, 51, 52, 53, 54, 55, 56, 57, 58, 59 and 60 is provided, wherein the amino acid sequences of CDR3 comprise between 0 and 7 amino acid modifications. In one embodiment the CDR3 regions comprise between 0 and 6, 0 and 5, 0 and 4, 0 and 3, 0 and 2 and 0 and 1 amino acid modifications. The modifications can be substitutions, deletions or insertions. In one embodiment, the modifications are substitutions.

In one embodiment, a single domain antibody comprising a complementary determining region 3 (CDR3) selected from the group consisting of SEQ ID NOs: 3, 6, 9, 12, 15, 18, 21, 24, 27, 30, 33, 36, 39, 40, 41, 42, 43, 44, 45, 46, 47, 48, 49, 50, 51, 52, 53, 54, 55, 56, 57, 58, 59 and 60 is provided, wherein the CDR3 regions of amino acid sequences of SEQ ID NOs: 3, 6, 21, 40, 41, 42, 43, 44, 45, 46, 47, 48, 49, 50, 51, 52, 53, 54, 55, 56, 57, 58, 59 and 60 comprise between 0 and 7 amino acid modifications, wherein the CDR3 regions of amino acid sequences of SEQ ID Nos: 12, 15, 18, 27, 30, 33 and 36 comprise between 0 and 5 amino acid modifications and wherein the CDR3 regions of amino acid sequences of SEQ ID NOs: 9, 24 and 39 comprise between 0 and 3 amino acid modifications.

In a preferred embodiment, the single domain antibody of the invention comprises complementary determining region 3 (CDR3) selected from the group consisting of 40, 41, 42, 43, 44, 45, 46, 47, 48, 49, 50, 51, 52, 53, 54, 55, 56, 57, 58, 59 and 60 wherein the amino acid sequences of CDR3 comprise between 0 and 7 amino acid modifications. In one embodiment, the complementary determining region 3 (CDR3) selected from the group consisting of 40, 41, 42, 43 and 44. In a most preferred embodiment, the complementary determining region 3 (CDR3) is SEQ ID NO: 41. In one embodiment the CDR3 regions comprise between 0 and 6, 0 and 5, 0 and 4, 0 and 3, 0 and 2 or 0 and 1 amino acid modifications. The modifications can be substitutions, deletions or insertions. In one embodiment, the modifications are substitutions.

In one embodiment the single domain antibody of the invention may further comprise a CDR2 region. The CDR2 region may be defined according to a SEQ ID NO disclosed herein. In a further embodiment, the single domain antibody of the invention may further comprise a CDR1 region and CDR2 region. The CDR1 region and the CDR2 region may be defined according to a SEQ ID NO disclosed herein. In each embodiment, the single domain antibody may further comprise four framework regions (FR1, FR2, FR3 and FR4).

In one aspect, an anti-SARS-CoV-2 single domain antibody is provided, wherein the single antibody domain comprises
(a) a CDR2 comprising SEQ ID NO:2 and a CDR3 comprising SEQ ID NO:3;
(b) a CDR2 comprising SEQ ID NO:5 and a CDR3 comprising SEQ ID NO:6;
(c) a CDR2 comprising SEQ ID NO:8 and a CDR3 comprising SEQ ID NO:9;
(d) a CDR2 comprising SEQ ID NO:11 and a CDR3 comprising SEQ ID NO:12;
(e) a CDR2 comprising SEQ ID NO:14 and a CDR3 comprising SEQ ID NO:15;
(f) a CDR2 comprising SEQ ID NO:17 and a CDR3 comprising SEQ ID NO:18;
(g) a CDR2 comprising SEQ ID NO:20 and a CDR3 comprising SEQ ID NO:21;
(h) a CDR2 comprising SEQ ID NO:23 and a CDR3 comprising SEQ ID NO:24;
(i) a CDR2 comprising SEQ ID NO:26 and a CDR3 comprising SEQ ID NO:27;
(j) a CDR2 comprising SEQ ID NO:29 and a CDR3 comprising SEQ ID NO:30;
(k) a CDR2 comprising SEQ ID NO:32 and a CDR3 comprising SEQ ID NO:33;
(l) a CDR2 comprising SEQ ID NO:35 and a CDR3 comprising SEQ ID NO:36;
(m) a CDR2 comprising SEQ ID NO:38 and a CDR3 comprising SEQ ID NO:39;
(n) a CDR2 comprising SEQ ID NO:2 and a CDR3 comprising SEQ ID NO:40;
(o) a CDR2 comprising SEQ ID NO:2 and a CDR3 comprising SEQ ID NO:41;
(p) a CDR2 comprising SEQ ID NO:2 and a CDR3 comprising SEQ ID NO:42;
(q) a CDR2 comprising SEQ ID NO:2 and a CDR3 comprising SEQ ID NO:43;
(r) a CDR2 comprising SEQ ID NO:2 and a CDR3 comprising SEQ ID NO:44;
(s) a CDR2 comprising SEQ ID NO:2 and a CDR3 comprising SEQ ID NO:45;
(t) a CDR2 comprising SEQ ID NO:2 and a CDR3 comprising SEQ ID NO:46;
(u) a CDR2 comprising SEQ ID NO:2 and a CDR3 comprising SEQ ID NO:47;
(v) a CDR2 comprising SEQ ID NO:2 and a CDR3 comprising SEQ ID NO:48;
(w) a CDR2 comprising SEQ ID NO:2 and a CDR3 comprising SEQ ID NO:49;
(x) a CDR2 comprising SEQ ID NO:2 and a CDR3 comprising SEQ ID NO:50;
(y) a CDR2 comprising SEQ ID NO:2 and a CDR3 comprising SEQ ID NO:51;
(z) a CDR2 comprising SEQ ID NO:2 and a CDR3 comprising SEQ ID NO:52;
(aa) a CDR2 comprising SEQ ID NO:2 and a CDR3 comprising SEQ ID NO:53;
(bb) a CDR2 comprising SEQ ID NO:2 and a CDR3 comprising SEQ ID NO:54;
(cc) a CDR2 comprising SEQ ID NO:2 and a CDR3 comprising SEQ ID NO:55;
(dd) a CDR2 comprising SEQ ID NO:2 and a CDR3 comprising SEQ ID NO:56;
(ee) a CDR2 comprising SEQ ID NO:2 and a CDR3 comprising SEQ ID NO:57;
(ff) a CDR2 comprising SEQ ID NO:2 and a CDR3 comprising SEQ ID NO:58;
(gg) a CDR2 comprising SEQ ID NO:2 and a CDR3 comprising SEQ ID NO:59;
(hh) a CDR2 comprising SEQ ID NO:2 and a CDR3 comprising SEQ ID NO:60;

wherein the amino acid sequence of CDR3 comprises between 0 and 7 amino acid modifications and wherein the amino acid sequence of CDR2 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications.

In one embodiment, an anti-SARS-CoV-2 single domain antibody is provided, wherein the single antibody domain comprises
(a) a CDR2 comprising SEQ ID NO:2 and a CDR3 comprising SEQ ID NO:40;
(b) a CDR2 comprising SEQ ID NO:2 and a CDR3 comprising SEQ ID NO:41;
(c) a CDR2 comprising SEQ ID NO:2 and a CDR3 comprising SEQ ID NO:42;
(d) a CDR2 comprising SEQ ID NO:2 and a CDR3 comprising SEQ ID NO:43;
(e) a CDR2 comprising SEQ ID NO:2 and a CDR3 comprising SEQ ID NO:44;
(f) a CDR2 comprising SEQ ID NO:2 and a CDR3 comprising SEQ ID NO:45;
(g) a CDR2 comprising SEQ ID NO:2 and a CDR3 comprising SEQ ID NO:46;
(h) a CDR2 comprising SEQ ID NO:2 and a CDR3 comprising SEQ ID NO:47;
(i) a CDR2 comprising SEQ ID NO:2 and a CDR3 comprising SEQ ID NO:48;
(j) a CDR2 comprising SEQ ID NO:2 and a CDR3 comprising SEQ ID NO:49;
(k) a CDR2 comprising SEQ ID NO:2 and a CDR3 comprising SEQ ID NO:50;
(l) a CDR2 comprising SEQ ID NO:2 and a CDR3 comprising SEQ ID NO:51;
(m) a CDR2 comprising SEQ ID NO:2 and a CDR3 comprising SEQ ID NO:52;
(n) a CDR2 comprising SEQ ID NO:2 and a CDR3 comprising SEQ ID NO:53;
(o) a CDR2 comprising SEQ ID NO:2 and a CDR3 comprising SEQ ID NO:54;
(p) a CDR2 comprising SEQ ID NO:2 and a CDR3 comprising SEQ ID NO:55;
(q) a CDR2 comprising SEQ ID NO:2 and a CDR3 comprising SEQ ID NO:56;
(r) a CDR2 comprising SEQ ID NO:2 and a CDR3 comprising SEQ ID NO:57;
(s) a CDR2 comprising SEQ ID NO:2 and a CDR3 comprising SEQ ID NO:58;
(t) a CDR2 comprising SEQ ID NO:2 and a CDR3 comprising SEQ ID NO:59;
(u) a CDR2 comprising SEQ ID NO:2 and a CDR3 comprising SEQ ID NO:60;

wherein the amino acid sequence of CDR3 comprises between 0 and 7 amino acid modifications and wherein the amino acid sequence of CDR2 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications.

In one embodiment, an anti-SARS-CoV-2 single domain antibody is provided, wherein the single antibody domain comprises
(a) a CDR2 comprising SEQ ID NO:2 and a CDR3 comprising SEQ ID NO:40;
(b) a CDR2 comprising SEQ ID NO:2 and a CDR3 comprising SEQ ID NO:41;
(c) a CDR2 comprising SEQ ID NO:2 and a CDR3 comprising SEQ ID NO:42;
(d) a CDR2 comprising SEQ ID NO:2 and a CDR3 comprising SEQ ID NO:43;
(e) a CDR2 comprising SEQ ID NO:2 and a CDR3 comprising SEQ ID NO:44;

wherein the amino acid sequence of CDR3 comprises between 0 and 7 amino acid modifications and wherein the amino acid sequence of CDR2 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications.

In one embodiment, an anti-SARS-CoV-2 single domain antibody is provided, wherein the single antibody domain comprises
(a) a CDR2 comprising SEQ ID NO:2 and a CDR3 comprising SEQ ID NO:41;
wherein the amino acid sequence of CDR3 comprises between 0 and 7 amino acid modifications and wherein the amino acid sequence of CDR2 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications.

In one embodiment the single domain antibody of the invention may further comprise a CDR1 region. The CDR1 region may be defined according to a SEQ ID NO disclosed herein. In each embodiment, the single domain antibody may further comprise four framework regions (FR1, FR2, FR3 and FR4).

In one aspect, an anti-SARS-CoV-2 single domain antibody is provided, wherein the single antibody domain comprises
(a) CDR1 comprising SEQ ID NO:1, a CDR2 comprising SEQ ID NO:2 and a CDR3 comprising SEQ ID NO:3;
(b) a CDR1 comprising SEQ ID NO:4, a CDR2 comprising SEQ ID NO:5 and a CDR3 comprising SEQ ID NO:6;
(c) a CDR1 comprising SEQ ID NO:7, a CDR2 comprising SEQ ID NO:8 and a CDR3 comprising SEQ ID NO:9;
(d) a CDR1 comprising SEQ ID NO:10, a CDR2 comprising SEQ ID NO:11 and a CDR3 comprising SEQ ID NO:12;
(e) a CDR1 comprising SEQ ID NO:13, a CDR2 comprising SEQ ID NO:14 and a CDR3 comprising SEQ ID NO:15;
(f) a CDR1 comprising SEQ ID NO:16, a CDR2 comprising SEQ ID NO:17 and a CDR3 comprising SEQ ID NO:18;
(g) a CDR1 comprising SEQ ID NO:19, a CDR2 comprising SEQ ID NO:20 and a CDR3 comprising SEQ ID NO:21;
(h) a CDR1 comprising SEQ ID NO:22, a CDR2 comprising SEQ ID NO:23 and a CDR3 comprising SEQ ID NO:24;
(i) a CDR1 comprising SEQ ID NO:25, a CDR2 comprising SEQ ID NO:26 and a CDR3 comprising SEQ ID NO:27;
(j) a CDR1 comprising SEQ ID NO:28, a CDR2 comprising SEQ ID NO:29 and a CDR3 comprising SEQ ID NO:30;
(k) a CDR1 comprising SEQ ID NO:31, a CDR2 comprising SEQ ID NO:32 and a CDR3 comprising SEQ ID NO:33;
(l) a CDR1 comprising SEQ ID NO:34, a CDR2 comprising SEQ ID NO:35 and a CDR3 comprising SEQ ID NO:36;
(m) a CDR1 comprising SEQ ID NO:37, a CDR2 comprising SEQ ID NO:38 and a CDR3 comprising SEQ ID NO:39;
(n) CDR1 comprising SEQ ID NO:1, a CDR2 comprising SEQ ID NO:2 and a CDR3 comprising SEQ ID NO:40;
(o) CDR1 comprising SEQ ID NO:1, a CDR2 comprising SEQ ID NO:2 and a CDR3 comprising SEQ ID NO:41;
(p) CDR1 comprising SEQ ID NO:1, a CDR2 comprising SEQ ID NO:2 and a CDR3 comprising SEQ ID NO:42;
(q) CDR1 comprising SEQ ID NO:1, a CDR2 comprising SEQ ID NO:2 and a CDR3 comprising SEQ ID NO:43;
(r) CDR1 comprising SEQ ID NO:1, a CDR2 comprising SEQ ID NO:2 and a CDR3 comprising SEQ ID NO:44;
(s) CDR1 comprising SEQ ID NO:1, a CDR2 comprising SEQ ID NO:2 and a CDR3 comprising SEQ ID NO:45;
(t) CDR1 comprising SEQ ID NO:1, a CDR2 comprising SEQ ID NO:2 and a CDR3 comprising SEQ ID NO:46;
(u) CDR1 comprising SEQ ID NO:1, a CDR2 comprising SEQ ID NO:2 and a CDR3 comprising SEQ ID NO:47;
(v) CDR1 comprising SEQ ID NO:1, a CDR2 comprising SEQ ID NO:2 and a CDR3 comprising SEQ ID NO:48;
(w) CDR1 comprising SEQ ID NO:1, a CDR2 comprising SEQ ID NO:2 and a CDR3 comprising SEQ ID NO:49;
(x) CDR1 comprising SEQ ID NO:1, a CDR2 comprising SEQ ID NO:2 and a CDR3 comprising SEQ ID NO:50;
(y) CDR1 comprising SEQ ID NO:1, a CDR2 comprising SEQ ID NO:2 and a CDR3 comprising SEQ ID NO:51;
(z) CDR1 comprising SEQ ID NO:1, a CDR2 comprising SEQ ID NO:2 and a CDR3 comprising SEQ ID NO:52;
(aa) CDR1 comprising SEQ ID NO:1, a CDR2 comprising SEQ ID NO:2 and a CDR3 comprising SEQ ID NO:53;
(bb) CDR1 comprising SEQ ID NO:1, a CDR2 comprising SEQ ID NO:2 and a CDR3 comprising SEQ ID NO:54;
(cc) CDR1 comprising SEQ ID NO:1, a CDR2 comprising SEQ ID NO:2 and a CDR3 comprising SEQ ID NO:55;
(dd) CDR1 comprising SEQ ID NO:1, a CDR2 comprising SEQ ID NO:2 and a CDR3 comprising SEQ ID NO:56;
(ee) CDR1 comprising SEQ ID NO:1, a CDR2 comprising SEQ ID NO:2 and a CDR3 comprising SEQ ID NO:57;
(ff) CDR1 comprising SEQ ID NO:1, a CDR2 comprising SEQ ID NO:2 and a CDR3 comprising SEQ ID NO:58;
(gg) CDR1 comprising SEQ ID NO:1, a CDR2 comprising SEQ ID NO:2 and a CDR3 comprising SEQ ID NO:59;
(hh) CDR1 comprising SEQ ID NO:1, a CDR2 comprising SEQ ID NO:2 and a CDR3 comprising SEQ ID NO:60;

wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications. In one embodiment the CDR1 regions comprise between 0 and 3, 0 and 2, 0 and 4, 0 and 1 amino acid modifications.

In one embodiment, an anti-SARS-CoV-2 single domain antibody is provided, wherein the single antibody domain comprises
(a) CDR1 comprising SEQ ID NO:1, a CDR2 comprising SEQ ID NO:2 and a CDR3 comprising SEQ ID NO:40;
(b) CDR1 comprising SEQ ID NO:1, a CDR2 comprising SEQ ID NO:2 and a CDR3 comprising SEQ ID NO:41;
(c) CDR1 comprising SEQ ID NO:1, a CDR2 comprising SEQ ID NO:2 and a CDR3 comprising SEQ ID NO:42;
(d) CDR1 comprising SEQ ID NO:1, a CDR2 comprising SEQ ID NO:2 and a CDR3 comprising SEQ ID NO:43;
(e) CDR1 comprising SEQ ID NO:1, a CDR2 comprising SEQ ID NO:2 and a CDR3 comprising SEQ ID NO:44;
(f) CDR1 comprising SEQ ID NO:1, a CDR2 comprising SEQ ID NO:2 and a CDR3 comprising SEQ ID NO:45;
(g) CDR1 comprising SEQ ID NO:1, a CDR2 comprising SEQ ID NO:2 and a CDR3 comprising SEQ ID NO:46;
(h) CDR1 comprising SEQ ID NO:1, a CDR2 comprising SEQ ID NO:2 and a CDR3 comprising SEQ ID NO:47;
(i) CDR1 comprising SEQ ID NO:1, a CDR2 comprising SEQ ID NO:2 and a CDR3 comprising SEQ ID NO:48;
(j) CDR1 comprising SEQ ID NO:1, a CDR2 comprising SEQ ID NO:2 and a CDR3 comprising SEQ ID NO:49;
(k) CDR1 comprising SEQ ID NO:1, a CDR2 comprising SEQ ID NO:2 and a CDR3 comprising SEQ ID NO:50;
(l) CDR1 comprising SEQ ID NO:1, a CDR2 comprising SEQ ID NO:2 and a CDR3 comprising SEQ ID NO:51;
(m) CDR1 comprising SEQ ID NO:1, a CDR2 comprising SEQ ID NO:2 and a CDR3 comprising SEQ ID NO:52;
(n) CDR1 comprising SEQ ID NO:1, a CDR2 comprising SEQ ID NO:2 and a CDR3 comprising SEQ ID NO:53;
(o) CDR1 comprising SEQ ID NO:1, a CDR2 comprising SEQ ID NO:2 and a CDR3 comprising SEQ ID NO:54;
(p) CDR1 comprising SEQ ID NO:1, a CDR2 comprising SEQ ID NO:2 and a CDR3 comprising SEQ ID NO:55;
(q) CDR1 comprising SEQ ID NO:1, a CDR2 comprising SEQ ID NO:2 and a CDR3 comprising SEQ ID NO:56;
(r) CDR1 comprising SEQ ID NO:1, a CDR2 comprising SEQ ID NO:2 and a CDR3 comprising SEQ ID NO:57;
(s) CDR1 comprising SEQ ID NO:1, a CDR2 comprising SEQ ID NO:2 and a CDR3 comprising SEQ ID NO:58;
(t) CDR1 comprising SEQ ID NO:1, a CDR2 comprising SEQ ID NO:2 and a CDR3 comprising SEQ ID NO:59;
(u) CDR1 comprising SEQ ID NO:1, a CDR2 comprising SEQ ID NO:2 and a CDR3 comprising SEQ ID NO:60;

wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications.

In one embodiment, an anti-SARS-CoV-2 single domain antibody is provided, wherein the single antibody domain comprises
(a) CDR1 comprising SEQ ID NO:1, a CDR2 comprising SEQ ID NO:2 and a CDR3 comprising SEQ ID NO:40;
(b) CDR1 comprising SEQ ID NO:1, a CDR2 comprising SEQ ID NO:2 and a CDR3 comprising SEQ ID NO:41;
(c) CDR1 comprising SEQ ID NO:1, a CDR2 comprising SEQ ID NO:2 and a CDR3 comprising SEQ ID NO:42;
(d) CDR1 comprising SEQ ID NO:1, a CDR2 comprising SEQ ID NO:2 and a CDR3 comprising SEQ ID NO:43;
(e) CDR1 comprising SEQ ID NO:1, a CDR2 comprising SEQ ID NO:2 and a CDR3 comprising SEQ ID NO:44;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In one embodiment, the CDR3 is SEQ ID NO: 41.

In one embodiment, an anti-SARS-CoV-2 single domain antibody is provided comprising:
a CDR1 comprising SEQ ID NO:1, a CDR2 comprising SEQ ID NO:2 and a CDR3 comprising an amino acid sequence selected from SEQ ID NOs: 40, 41, 42, 43, 44, 45, 46, 47, 48, 49, 50, 51, 52, 53, 54, 55, 56, 57, 58, 59 and 60.

In one embodiment, an anti-SARS-CoV-2 single domain antibody is provided comprising:
a CDR1 comprising SEQ ID NO:1, a CDR2 comprising SEQ ID NO:2 and a CDR3 comprising an amino acid sequence selected from SEQ ID NOs: 40, 41, 42, 43 and 44. In one embodiment, an anti-SARS-CoV-2 single domain antibody is provided comprising: a CDR1 comprising SEQ ID NO:1, a CDR2 comprising SEQ ID NO:2 and a CDR3 comprising SEQ ID NOs: 41.

In one embodiment, the single chain antibody comprises four framework regions. The framework regions separate the CDR sequences. In one embodiment, the four framework regions are framework region 1 (FR1), framework region 2 (FR2), framework region 3 (FR3) and framework region 4 (FR4) and are interspersed between the CDR1, CDR2 and CDR3 (i.e FR1-CDR1-FR2-CDR2-FR3-CDR3-FR4). In one embodiment, the single domain antibody of the invention comprises or essentially consists of four framework regions (FR1, FR2, FR3 and FR4) and three CDRs (CDR1, CDR2 and CDR3). In one embodiment, the single domain antibody of the invention consists of four framework regions (FR1, FR2, FR3 and FR4) and three CDRs (CDR1, CDR2 and CDR3).

In some embodiments, the one or more amino acid modifications are in the CDR region or regions. In other embodiments, the one or more amino acid modifications are in the framework regions, i.e. not in the CDR region or regions. In some embodiments, the one or more polynucleotide modifications are in the CDR region or regions. In other embodiments, the one or more polynucleotide modifications are in the framework regions, i.e. not in the CDR region or regions.

In one embodiment the CDR3 regions comprise between 0 and 7, 0 and 6, 0 and 5, 0 and 4, 0 and 3, 0 and 2 or 0 and 1 amino acid modifications. In one embodiment the CDR2 regions comprise 0 and 4, 0 and 3, 0 and 2 or 0 and 1 amino acid modifications. In one embodiment the CDR1 regions comprise 0 and 4, 0 and 3, 0 and 2 or 0 and 1 amino acid modifications. The modifications can be substitutions, deletions or insertions. In one embodiment, the modifications are substitutions.

In one embodiment, a single domain antibody of the invention comprising one or more modifications has a binding affinity for the receptor binding domain of the SARS-CoV-2 S-protein that is substantially equal to, or better than (for example, a lower Kd value) than the specified sequence without any modifications.

In one aspect, an anti-SARS-CoV-2 single domain antibody comprising an amino acid sequence having at least 70% identity to a sequence selected from the group consisting of: SEQ 61, 62, 63, 64, 65, 66, 67, 68, 69, 70, 71, 72, 73, 87, 88, 89, 90, 91, 92, 93, 94, 95, 96, 97, 98, 99, 101, 102, 103, 104, 105, 106 and 107 is provided. Each of these sequences comprises three CDR regions (CDR1, CDR2 and CDR3) and four framework regions (FR1, FR2, FR3 and FR4). In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: SEQ 61, 62, 63, 64, 65, 66, 67, 68, 69, 70, 71, 72, 73, 87, 88, 89, 90, 91, 92, 93, 94, 95, 96, 97, 98, 99, 101, 102, 103, 104, 105, 106 and 107. In one embodiment, an anti-SARS-CoV-2 single domain antibody comprising SEQ 61, 62, 63, 64, 65, 66, 67, 68, 69, 70, 71, 72, 73, 87, 88, 89, 90, 91, 92, 93, 94, 95, 96, 97, 98, 99, 101, 102, 103, 104, 105, 106 and 107 is provided. In one embodiment, an anti-SARS-CoV-2 single domain antibody consisting or essentially consisting of SEQ 61, 62, 63, 64, 65, 66, 67, 68, 69, 70, 71, 72, 73, 87, 88, 89, 90, 91, 92, 93, 94, 95, 96, 97, 98, 99, 101, 102, 103, 104, 105, 106 and 107 is provided.

At least herein and throughout means, in some embodiments, the recited percentage up to 100%. For example, at least 75% can mean, in some embodiments, 75% to 100%.

In one embodiment, an anti-SARS-CoV-2 single domain antibody comprising an amino acid sequence having at least 70% identity to a sequence selected from the group consisting of: SEQ ID NO: 87, 88, 89, 90, 91, 92, 93, 94, 95, 96, 97, 98, 99, 101, 102, 103, 104, 105, 106 and 107 is provided. Each of these sequences comprises three CDR regions (CDR1, CDR2 and CDR3) and four framework regions (FR1, FR2, FR3 and FR4), wherein the CDR3 region has been affinity matured. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: SEQ 87, 88, 89, 90, 91, 92, 93, 94, 95, 96, 97, 98, 99, 101, 102, 103, 104, 105, 106 and 107. In one embodiment, an anti-SARS-CoV-2 single domain antibody comprising SEQ 87, 88, 89, 90, 91, 92, 93, 94, 95, 96, 97, 98, 99, 101, 102, 103, 104, 105, 106 and 107 is provided. In one embodiment, an anti-SARS-CoV-2 single domain antibody consisting or essentially consisting of SEQ 87, 88, 89, 90, 91, 92, 93, 94, 95, 96, 97, 98, 99, 101, 102, 103, 104, 105, 106 and 107 is provided.

In one embodiment, an anti-SARS-CoV-2 single domain antibody comprising an amino acid sequence having at least 70% identity to a sequence selected from the group consisting of: SEQ ID NOs: 87, 88, 89, 90 and 91. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: of SEQ ID NOs: 87, 88, 89, 90 and 91. In one embodiment, the amino acid sequence is selected from the group consisting of SEQ ID NOs: 87, 88, 89, 90 and 91.

In one embodiment, an anti-SARS-CoV-2 single domain antibody comprising an amino acid sequence having at least 70% identity to SEQ ID NOs: 88. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 88. In one embodiment, the amino acid sequence is SEQ ID NO: 88.

In one aspect, a polynucleotide sequence is provided encoding a single domain antibody of the invention. In one embodiment, the polynucleotide is DNA or RNA. Such nucleic acid sequences may be in the form of a genetic construct.

In one embodiment, an anti-SARS-CoV-2 single domain antibody comprising a polynucleotide sequence having at least 70% identity to a sequence selected from the group consisting of: SEQ 74, 75, 76, 77, 78, 79, 80, 81, 82, 83, 84, 85, 86, 108, 109, 110, 111, 112, 113, 114, 115, 116, 117, 118, 119, 120, 121, 122, 123, 124, 125, 126, 127 and 128 is provided. In one embodiment, the polynucleotide sequence has at least 75%, 80%, 85%, 90%, 91%, 92%, 93%, 94%, 95%, 96%, 97%, 98% or at least 99% identity to a sequence selected from the group consisting of: SEQ 74, 75, 76, 77, 78, 79, 80, 81, 82, 83, 84, 85, 86, 108, 109, 110, 111, 112, 113, 114, 115, 116, 117, 118, 119, 120, 121, 122, 123, 124, 125, 126, 127 and 128. In one embodiment, an anti-SARS-CoV-2 single domain antibody comprising SEQ 74, 75, 76, 77, 78, 79, 80, 81, 82, 83, 84, 85, 86, 108, 109, 110, 111, 112, 113, 114, 115, 116, 117, 118, 119, 120, 121, 122, 123, 124, 125, 126, 127 and 128 is provided. In one embodiment, an anti-SARS-CoV-2 single domain antibody consisting or essentially consisting of 74, 75, 76, 77, 78, 79, 80, 81, 82, 83, 84, 85, 86, 108, 109, 110, 111, 112, 113, 114, 115, 116, 117, 118, 119, 120, 121, 122, 123, 124, 125, 126, 127 and 128 is provided.

In one embodiment, an anti-SARS-CoV-2 single domain antibody comprising a polynucleotide sequence having at least 70% identity to a sequence selected from the group consisting of: SEQ ID NO: 108, 109, 110, 111, 112, 113, 114, 115, 116, 117, 118, 119, 120, 121, 122, 123, 124, 125, 126, 127 and 128 is provided. In one embodiment, the polynucleotide sequence has at least 75%, 80%, 85%, 90%, 91%, 92%, 93%, 94%, 95%, 96%, 97%, 98% or at least 99% identity to a sequence selected from the group consisting of: SEQ ID NO: 108, 109, 110, 111, 112, 113, 114, 115, 116, 117, 118, 119, 120, 121, 122, 123, 124, 125, 126, 127 and 128. In one embodiment, an anti-SARS-CoV-2 single domain antibody comprising SEQ 108, 109, 110, 111, 112, 113, 114, 115, 116, 117, 118, 119, 120, 121, 122, 123, 124, 125, 126, 127 and 128 is provided. In one embodiment, an anti-SARS-CoV-2 single domain antibody consisting or essentially consisting of SEQ 108, 109, 110, 111, 112, 113, 114, 115, 116, 117, 118, 119, 120, 121, 122, 123, 124, 125, 126, 127 and 128 is provided.

In one embodiment, an anti-SARS-CoV-2 single domain antibody comprising a polynucleotide sequence having at least 70% identity to a sequence selected from the group consisting of SEQ ID NOs: 108, 109, 110, 111 and 112. In one embodiment, the polynucleotide sequence has at least 75%, 80%, 85%, 90%, 91%, 92%, 93%, 94%, 95%, 96%, 97%, 98% or at least 99% identity to a sequence selected from the group consisting of SEQ ID NOs: 108, 109, 110, 111 and 112. In one embodiment, the polynucleotide sequence is selected from the group consisting of SEQ ID NOs: 108, 109, 110, 111 and 112. In one embodiment, the polynucleotide sequence is SEQ ID NO: 109.

The single domain antibodies of the invention bind to the receptor binding domain of the SARS-CoV-2 S-protein. In one embodiment, the single domain antibodies of the invention block or modulate the binding between the receptor binding domain of a coronavirus, in particular the SARS-CoV-2 spike (S) protein, and the angiotensin converting enzyme 2 receptor (ACE2 receptor). In one embodiment, the single domain antibodies of the invention inhibit binding of the receptor binding domain of the SARS-CoV-2 spike (S) protein to the ACE2 receptor, wherein binding of the receptor binding domain of the SARS-CoV-2 spike (S) protein to the ACE2 receptor is inhibited by at least 10%, optionally at least 20%, at least 30%, at least 40%, at least 50%, at least 60%, at least 70%, at least 80%, at least 90%, at least 95% or 100%. Percentage inhibition of binding to the ACE2 receptor can be measured in numerous ways, as well understood by the skilled person, including but not limited to surface plasmon resonance.

By interfering with the interaction between a coronavirus spike protein and its target, the single domain antibodies of the invention can neutralize coronavirus infection. In one embodiment the single domain antibodies of the invention can neutralize SARS-CoV-2 infection. In one embodiment, the single domain antibodies have an ND50 value (ND50=concentration of antibody that reduces the number of infected cells by 50%) of less than 100 μM, less than 10 μM, less than 5 μM, less than 1 μM, less than 0.5 μM, less than 0.1 μM or less than 0.01 μM.

In one embodiment, the single domain antibodies have an ND50 value of less than 10 0nM less than 10 nM, less than 5 nM, less than 1 nM, less than 0.5 nM or less than 0.1 nM. In one embodiment, the single domain antibodies have an ND50 value of less than 0.1 nM less than 10 pM, less than 5 pM, less than 1 pM, less than 0.5 pM or less than 0.1 pM. The ND50 value can be determined using any standard neutralization assay, including that disclosed herein. In one embodiment the single domain antibodies of the invention can prevent non-neutralized SARS-CoV-2 infection from spreading.

In one embodiment, the single domain antibodies of the invention have a Kd value for SARS-CoV-2 spike protein, in particular the receptor binding domain of the spike protein, of less than 100 nM, less than 50 nM, less than 20 nM, less than 10 nM, less than 9 nM, less than 8 nM, less than 7 nM, less than 6 nM, less than 5 nM, less than 4 nM, less than 3 nM, less than 2 nM, less than 1 nM, less than 0.5 nM or less than 0.1 nM. In one embodiment, the single domain antibodies of the invention have a Kd value for SARS-CoV-2 spike protein of less than 100 pM less than 10 pM, less than 5 pM, less than 1 pM, less than 0.5 pM or less than 0.1 pM. Binding affinity of an antibody can be measured according to several standard well-known techniques, including for example surface plasma resonance. In one embodiment, a single domain antibody of the invention having one or more modifications as specified herein has a binding affinity value that is within 20% (i.e. within the range of 20% below or 20% above the binding affinity value of the corresponding single domain antibody without one or more modifications) of the binding affinity value of the corresponding single domain antibody without one or more modifications. In one embodiment, the binding affinity value of single domain antibody of the invention having one or more modifications as specified herein is within 10%, optionally 5%, 4%, 3%, 2% or 1% of the binding affinity value of the corresponding single domain antibody without one or more modifications.

Furthermore, the single domain antibodies of the invention can modulate, reduce or prevent coronavirus infectivity. The single domain antibodies or pharmaceutical compositions of the invention can modulate, block or inhibit the fusion of a coronavirus to a target host cell. The single domain antibodies or pharmaceutical compositions of the invention can modulate, block or inhibit entry of coronavirus into a target host cell.

In one aspect, an affinity matured mutant of a single domain antibody of the invention is provided. In one embodiment, the CDR1 of the single domain antibody of the invention is affinity matured. In one embodiment, the CDR2 of the single domain antibody of the invention is affinity matured. In one embodiment, the CDR3 of the single domain antibody of the invention is affinity matured. In one embodiment, CDR3 is affinity matured and either CDR1 or CDR2 are also affinity matured. In one embodiment, CDR3 is affinity matured and CDR2 is also affinity matured. In one embodiment, CDR3 is affinity matured and CDR1 is also affinity matured. In one embodiment, each of CDR1, CDR2 and CDR3 are affinity matured. In one embodiment, at least one, at least two, at least three or all four of the framework regions (FR1, FR2, FR3 and FR4) are affinity matured. In one embodiment, each of CDR1, CDR2, CDR3, FR1, FR2, FR3 and FR4 are affinity matured. In one embodiment, the affinity of the affinity matured mutant of a single domain antibody of the invention has a higher affinity for SARS-CoV-2 receptor binding domain (RBD) than the parental antibody from which it was derived.

The CDR3 sequences of SEQ ID NOs: 40, 41, 42, 43, 44, 45, 46, 47, 48, 49, 50, 51, 52, 53, 54, 55, 56, 57, 58, 59 and 60 are affinity matured variants of the CDR3 sequence SEQ ID NO: 3 (NbRBD_H11). It has been surprisingly discovered that variation in an seven amino acid long region of the CDR3 region of a single domain antibody of the invention results in particularly improved affinity for SARS-CoV-1 spike protein, wherein the seven amino acid sequence starts at the second amino acid in the CDR3 sequence and ends at the eight amino acid in the sequence (i.e. position 1 refers to the starting alanine (A) in the CDR3 sequence). In one embodiment, the CDR3 region comprises modifications in a seven amino acid long region of the CDR3, wherein the seven amino acid long region starts at position 2 of the CDR3 and ends at position 8 of the CDR3.

In one aspect, a humanized single domain antibody of the invention is provided. Humanization requires the modification of the amino acid sequence of the antibody. Methods to reduce the immunogenicity of the single domain antibodies of the invention include CDR grafting on to a suitable antibody framework scaffold or remodelling variable surface residues, e.g. by site-directed mutagenesis. Methods of humanization of Nanobodies® are known to the skilled person, see for example Vincke et al., 2009. In one embodiment, the CDR1 of the single domain antibody of the invention is humanized. In one embodiment, the CDR2 of the single domain antibody of the invention is humanized. In one embodiment, the CDR3 of the single domain antibody of the invention is humanized. In one embodiment, at least one or at least two of the CDR1, CDR2 and CDR3 are humanized. In one embodiment, each of CDR1, CDR2 and CDR3 are humanized. In one embodiment, at least one, at least two, at least three or all four of the framework regions (FR1, FR2, FR3 and FR4) are humanized. In one embodiment, each of CDR1, CDR2, CDR3, FR1, FR2, FR3 and FR4 are humanized. In some embodiments, the single domain antibodies are conservatively humanised, for example to retain better antigen binding.

In one embodiment, the single domain antibody of the invention is fused to or conjugated to an Fc region. In one embodiment, the single domain antibody of the invention is fused to or conjugated to a scFv region. In one embodiment, the single domain antibody of the invention is a bivalent or polyvalent antibody. In one embodiment, the single domain antibody of the invention is bispecific or multispecific. In one embodiment, the single domain antibody is a chimeric antibody. In one embodiment, the single domain antibody comprises two VHH chains, wherein the VHH chains can be any of the single domain antibodies disclosed herein. In one embodiment, immunologically active molecules (antibodies, polypetpides or immunoglobulin molecules are provided) comprising the sequences of the invention, for example immunoglobulin isotypes (e.g., IgG, IgE, IgM, IgD and IgA) and their isotypic subclasses; fragments which comprise an antigen binding domain, such as Fab, F(ab′)2, Fv, scFv, dAb, Fd; and diabodies. In one embodiment, proteins or fusion expression products comprising the single domain antibodies of the invention are provided.

In one embodiment, a vector suitable for expressing a single domain antibody sequence of the invention is provided. The vector may be a plasmid, viral vector, cosmid, phage or artificial chromosome. In one aspect, a host cell comprising an expression vector or plasmid, wherein the expression vector or plasmid comprises a polynucleotide of the invention is provided. In one embodiment, the host cell comprises a polynucleotide of the invention integrated within the genome of the host cell. In one embodiment, the host cell is a prokaryotic cell, for example a bacterial cell, or a eukaryotic cell, for example a yeast cell or mammalian cell. In one embodiment, the host cell is Escherichia coli or CHO cells.

In one aspect, a method for producing a single domain antibody of the invention is provided comprising the steps of (a) culturing a host cell as provided herein under conditions suitable for producing a single domain antibody to obtain a culture containing single domain antibodies and (b) isolating said single domain antibodies from the culture.

One aspect of the invention provides single domain antibodies of the invention as defined above in a composition or pharmaceutical composition. The compositions may comprise, consist essentially of or consist of the single domain antibodies of the invention.

In one embodiment, a pharmaceutical composition comprising single domain antibodies of the invention is provided. The pharmaceutical compositions may be for human or animal usage in human and veterinary medicine. The pharmaceutical composition may be formulated according to route of administration. In one embodiment, the pharmaceutical composition is formulated for oral, nasal, ocular, buccal, vaginal, rectal, transdermal, intravenous, intramuscular or subcutaneous administration. In a preferred route of administration, the pharmaceutical composition is formulated for administration by inhalation, optionally nasal and or oral inhalation. Pharmaceutical compositions in this form may include aerosols, fine particles or dust.

In one embodiment, the composition or pharmaceutical composition optionally comprises one or more pharmaceutically acceptable excipients. In one embodiment, the composition or pharmaceutical composition optionally comprises one or more pharmaceutically acceptable adjuvants. In one embodiment, the composition or pharmaceutical composition is optionally admixed with one or more pharmaceutically acceptable diluents, excipients or carriers. Examples of such suitable excipients for the different forms of pharmaceutical compositions described herein may be found in the “Handbook of Pharmaceutical Excipients, 2^{nd} Edition, (1994), Edited by A Wade and P J Weller.

The composition or pharmaceutical composition may comprise one or more additional components. In one embodiment, the composition or pharmaceutical composition additionally comprises a pharmaceutically acceptable carrier. In one embodiment, the carrier is suitable for pulmonary delivery. In one embodiment, the composition or pharmaceutical composition additionally comprises a therapeutically active agent.

In one embodiment, the composition or pharmaceutical composition may be joined or conjugated to a protein or biologically active molecule. In one embodiment, the composition or pharmaceutical composition is part of a fusion protein and fused to one or more proteins or biologically active molecules. The protein or biologically active molecule may be a fluorescent protein, a bioluminescent protein, a split fluorescent protein (i.e split into two or more parts that will join together in the presence of drug), a split bioluminescent protein, a biosensor, a fluorescent biosensor or a split or hinged biosensor.

In one embodiment, a vaccine comprising single domain antibodies of the invention is provided. In one embodiment, the vaccine comprises a polynucleotide encoding a single domain antibody of the invention is provided.

The compositions, pharmaceutical compositions and vaccines of the invention can elicit an immune response in a subject, preferably an immune response to SARS-CoV-2. In some embodiments, the immune response is a protective immune response. In some embodiments, the immune response that reduces the symptoms or severity of SARS-CoV-2 in a subject.

In some embodiments, administration of the antibodies of the present invention, including but not limited to H11-H4-Fc, prevents or substantially reduces non-neutralised virus from replicating and/or spreading. This is supported by plaque reduction neutralisation test data: in the presence of antibodies of the present invention, including but not limited to H11-H4-Fc, plaques formed are smaller than would normally be expected (FIG. 9). In some embodiments, antibodies of the present invention, including but not limited to H11-H4-Fc, are capable of forming plaques that are 5% smaller than in the presence of a positive control, for example CR3022. In some embodiments, the plaques are 10% smaller than in the presence of a positive control (for example CR3022); in some embodiments, plaques are 15%, 20%, 25%, 30%, 35%, 40%, 45%, 50%, 55%, 60%, 65%, 70%, 75%, 80%, 85%, 90%, or 95% smaller than in the presence of a positive control (for example CR3022).

A pharmaceutical device, for example an inhaler, suitable to administer the pharmaceutical compositions of the invention is also provided. In one embodiment, the pharmaceutical device, for example an inhaler, comprises a single domain antibody of the invention.

A kit providing single domain antibodies of the invention is also provided. Such kits may include instructions for use and/or additional pharmaceutically active components. The single domain antibodies and the additional pharmaceutically active components may be formulated together, or alternatively in some embodiments, the single domain antibodies and the additional pharmaceutically active components may be present separately in the kit.

In one aspect, there is provided a single domain antibody of the invention or a pharmaceutical composition of the invention for use in medicine. The single domain antibodies or pharmaceutical compositions of the invention can be used to treat a coronavirus, optionally Middle Eastern respiratory syndrome (MERS-CoV) or severe acute respiratory syndrome coronavirus 1 (SARS-CoV-1), preferably COVID-19. The single domain antibodies or pharmaceutical compositions of the invention can be used to block or modify the interaction of the spike protein of a coronavirus, in particular SARS-CoV-2, with its target, angiotensin converting enzyme 2 receptor. In one embodiment, the single domain antibodies or pharmaceutical compositions of the invention block, reduce or inhibit binding of the spike protein of a coronavirus, in particular SARS-CoV-2, with its target, angiotensin converting enzyme 2 (ACE2) receptor. By interfering with the interaction between the spike protein and its target, the single domain antibodies or pharmaceutical compositions of the invention can neutralize coronavirus and/or can modulate, reduce or prevent coronavirus infectivity. The single domain antibodies or pharmaceutical compositions of the invention can modulate, block or inhibit the fusion of coronavirus to a target host cell. The single domain antibodies or pharmaceutical compositions of the invention can modulate, block or inhibit entry of coronavirus into a target host cell.

The single domain antibodies of the invention or pharmaceutical compositions of the invention can be used for the treatment or prophylaxis of coronavirus infection, in particular COVID-19. In one embodiment, there is provided a single domain antibody of the invention or a pharmaceutical composition of the invention for use in the treatment or prophylaxis of a coronavirus infection optionally Middle Eastern respiratory syndrome (MERS-CoV) or severe acute respiratory syndrome coronavirus 1 (SARS-CoV-1), preferably COVID-19. In one embodiment, there is provided a single domain antibody of the invention or a pharmaceutical composition of the invention for use in the treatment or prophylaxis of COVID-19.

In one aspect, a method for the treatment of a coronavirus in a subject is provided, comprising administering to a subject a therapeutically active amount of a single domain antibody of the invention. In one embodiment the subject is a mammal, preferably a human.

In one aspect, the use of a single domain antibody of the invention in the manufacture of a medicament for use in the treatment and/or prevention of a coronavirus is provided. In one embodiment, the use of a single domain antibody of the invention in the manufacture of a medicament for use in the treatment of a coronavirus is provided.

In one embodiment, the coronavirus is selected from the group consisting of MERS-CoV, SARS-CoV-1 and COVID-19. In one embodiment, the coronavirus is COVID-19. The invention may relate to treating a subject displaying severe symptoms of COVID-19 or alternatively to treating a subject showing milder symptoms of COVID-19. In some embodiments, the single domain antibodies of the invention are useful for treating a cytokine storm associated with a coronavirus infection.

In one embodiment, methods for the detection of a coronavirus protein, such as MERS-CoV, SARS-CoV-1 and SARS-CoV-2 are provided. In a preferred embodiment, a method for the detection of a SARS-CoV-2 protein is provided. In one embodiment, a method for detecting the presence of a coronavirus S-protein is provided. In one embodiment, a method for a method for detecting the presence of a SARS-CoV-2 S-protein is provided.

In one embodiment, a method for detecting a coronavirus protein in a sample is provided, wherein the method comprises the steps of (a) contacting a sample with the single domain antibodies of the invention and (b) detecting the antibody-antigen complex, wherein the presence of the complex indicates the presence of coronavirus protein. In step (a) of the method the sample is contacted with the single domain antibodies under suitable conditions for an antibody-antigen complex to form. The antigen is the coronavirus protein. In one embodiment, a method for detecting the presence of a coronavirus S-protein is provided. In one embodiment, a method for a method for detecting the presence of a SARS-CoV-2 S-protein, optionally the receptor binding domain of the S-protein, is provided.

The sample can be a biological sample, optionally a bodily fluid such as blood, serum, nasal secretions, sputum, plasma, urine or spinal fluid. In one embodiment the biological sample is bodily fluid obtained using a throat or nasal swab. In one embodiment, the biological sample is a tissue sample. The sample can be obtained from or isolated from a mammal, preferably a human. In one embodiment, the sample is obtained from or isolated from a subject who is suspected to have coronavirus.

Detecting the presence of coronavirus protein in a sample from a subject provides a positive indication that the subject is infected with coronavirus. In one embodiment, the results of the method of detection are used to diagnose a subject in relation to coronavirus. The presence of coronavirus protein in the method of detection would provide a positive diagnosis for coronavirus. The method of detection may also be used to provide a prediction of outcome in relation to infection of coronavirus infection.

In one embodiment, a method for detecting coronavirus protein in a subject is provided, wherein the method comprises the steps of (a) administering to a subject a single domain antibody of the invention and (b) detecting the presence of an antibody-antigen complex, wherein the presence of the complex indicates the presence of coronavirus protein in the subject. In one embodiment, a method for detecting coronavirus protein in a subject is provided, wherein the method comprises the steps of (a) administering to a subject a single domain antibody of the invention, (b) obtaining a sample from a subject and contacting the sample with a single domain antibody of the invention and (c) detecting the antibody-antigen complex, wherein the presence of the complex indicates the presence of coronavirus protein in the subject. The antigen is the coronavirus protein. In one embodiment, a method for detecting coronavirus protein in a subject is provided, wherein the method comprises the steps of (a) obtaining a sample from a subject, (b) contacting a sample from the subject with a single domain antibody of the invention and (c) detecting the antibody-antigen complex, wherein the presence of the complex indicates the presence of coronavirus protein in the subject. The sample may be an isolated sample (i.e. previously obtained from a subject).

In one aspect, a method for diagnosing coronavirus infection in a subject is provided, wherein the method comprises the steps of (a) administering to a subject a single domain antibody of the invention and (b) detecting the presence of an antibody-antigen complex, wherein the presence of the complex provides a positive diagnosis of coronavirus in the subject. In one embodiment, a method for diagnosing coronavirus infection in a subject is provided, wherein the method comprises the steps of (a) administering to a subject a single domain antibody of the invention, (b) obtaining a sample from a subject and contacting the sample with a single domain antibody of the invention and (c) detecting the antibody-antigen complex, wherein the presence of the complex provides a positive diagnosis of coronavirus in the subject. In one embodiment, a method for diagnosing coronavirus infection in a subject is provided, wherein the method comprises the steps of (a) obtaining a sample from a subject, (b) contacting a sample from the subject with a single domain antibody of the invention and (c) detecting the antibody-antigen complex, wherein the presence of the complex provides a positive diagnosis of coronavirus in the subject. In one embodiment, a method for diagnosing coronavirus infection in a subject, the method comprising (a) contacting a sample with a single domain antibody of the invention, (b) detecting the number of antibody-polypeptide complexes and (c) detecting the presence of coronavirus in the sample, wherein the presence of the complex provides a positive diagnosis of coronavirus in the subject. The sample may be an isolated sample (i.e. previously obtained from a subject).

In one embodiment, the method comprises the step of comparing the sample with reference sample values for levels of the antibody-antigen complex. An antigen-antibody complex value above that of the reference sample value can provide a positive indication of coronavirus infection. The sample may be an isolated sample (i.e. previously obtained from a subject).

In some embodiments the single domain antibody of the invention, may further comprise a marker such as a radiolabelled marker, imaging marker, MRI-marker, fluorescent marker or other detectable marker. Such antibodies can be used in each of the detection or diagnosis methods described herein to enable the detection of the antibody in the subject in real time. Such antibodies can also be used in each of the detection or diagnosis methods described herein to enable the detection of the antibody in a sample, such as a tissue or blood sample, isolated or obtained from a subject.

In one embodiment, an assay to detect a coronavirus is provided, wherein the assay comprises (a) contacting a sample obtained from a patient with a single domain antibody of the invention, wherein the single domain antibody comprises a detectable label or reporter molecule to selectively isolate the coronavirus in the patient sample. In one embodiment, an assay to detect coronavirus is provided, wherein the assay comprises (a) contacting a sample obtained from a patient with a fusion protein comprising a single domain antibody of the invention and a biosensor, optionally a fluorescent or hinged biosensor, The assay may for example be an enzyme-linked immunosorbent assay (ELISA), an immunofluorescence assay, a radioimmunoassay (RIA) or a fluorescence-activated cell sorting (FACS). The detectable label or reporter molecule can be a fluorescent or chemical molecule (e.g. fluorescein isothiocyanate, or rhodamine), a biosensor, a radioisotope or enzyme (e.g. alkaline phosphatase, β-galactosidase, horseradish peroxidase or luciferase).

In one embodiment a kit is provided, wherein the kit comprises (a) a detectable marker (b) a single domain antibody of the invention. The detectable label or reporter molecule can be a fluorescent or chemical molecule (e.g. fluorescein isothiocyanate, or rhodamine), a biosensor, optionally a fluorescent or hinged biosensor or a radioisotope or enzyme (e.g. alkaline phosphatase, β-galactosidase, horseradish peroxidase or luciferase).

The methods described herein can be in vitro or ex vivo. The methods described herein can also be performed in vivo.

The invention is described by reference to the following non-limiting Examples.

### EXAMPLES

### Methods

### Protein Production

The gene encoding amino acids 1-1208 of the SARS-CoV-2 spike glycoprotein ectodomain, with mutations of RRAR >GSAS at residues 682-685 (the furin cleavage site) and KV>PP at residues 986-987, as well as inclusion of a T4 fibritin trimerization domain, an HRV 3C cleavage site, a 6×His tag and a Twin-Strep-tag at the C-terminus {Wrapp et al., 2020} was synthesized and subcloned into a pHL-sec vector between the Agel and Xhol restriction sites. The validity of the clone was confirmed by sequencing.

Recombinant Spike ectodomain was expressed by transient transfection in HEK293S GnTI⁻ cells (ATCC CRL-3022) for 9 days at 30° C. Conditioned media was dialysed against 2×PBS buffer. The Spike ectodomain was purified by immobilised metal affinity chromatography using Talon resin (Takara Bio) charged with cobalt followed by size exclusion chromatography using HiLoad 16/60 Superdex 200 column in 150 mM NaCl, 10 mM HEPES (pH 8.0), 0.02% NaN₃ at 4° C. The final fractions containing the Spike ectodomain were identified by reducing SDS-PAGE, pooled, concentrated using a 100 kDa MWCO concentrator (Amicon Ultra, Merck), and stored at 16° C.

Codon optimised Genblocks (IDT Technology) for the receptor binding domain (RBD amino acids 330-532) of SARS-CoV2 (Genbank MN908947), and human Angiotensin Converting Enzyme 2 (ACE-2 amino acids 19-615) were inserted into the vector pOPINTTGneo (Nettleship, Watson et al. 2015) incorporating a C-terminal BirA-His6 tag and pOPINTTGneo-3C-Fc to make C-terminal fusions to Human IgG Fc. Recombinant RBDs and CR3022 Fab fragments were transiently expressed in Expi293™ (ThermoFisher Scientific, UK) and proteins were purified from culture supernatants by immobilised metal affinity using an automated protocol implemented on an AKTAxpress (GE Healthcare, UK) (Nettleship, Rahman-Huq et al. 2009) followed by a Superdex 200 10/300GL column, using phosphate-buffered saline (PBS) pH 7.4 buffer. Purified protein were biotinylated in vitro by incubation with biotin protein ligase (Avidity LLC, Co, USA).

### Affinity Maturation of NbNH11

Mutations in the CDR3 of NbNH11 were introduced by PCR using seven pairs of forward and reverse primers forward primers (H11_AM_CDR3_F1-7 in combination with H11_AM_CDR3_R1-7). The mutated fragments were amplified with the primers H11_Phd_F and H11_Phd_R (Table 3a), digested with Sfil restriction enzyme and cloned into pADL-23c phagemid (Antibody Design Laboratories, San Diego CA, USA). The ligated vector was transformed into TG1 cells by electroporation to give a phage library consisting approximately 2×10⁹ independent clones. Two rounds of biopanning of the library were carried out on 5 nM and 1 nM of RBD, respectively, as described above and positive phage identified by ELISA and sequenced.

TABLE 3a
PCR primers used for affinity maturation

| Primer name | Sequence |
|---|---|
| Forward primers | |
| H11_AM_CDR3_F1 | 5′GCCGTTTATTACTGTGCANNKNNKNNKNNKN |
| | NKCGGTCCCTCCTTAGCGAC-3′ |
| H11_AM_CDR3_F2 | 5′-GTTTATTACTGTGCACAAACGNNKNNKNNK |
| | NNKNNKCTCCTTAGCGACTATGCC-3′ |
| H11_AM_CDR3_F3 | 5′-CTGTGCACAAACGCGGGTCNNKNNKNNKNN |
| | KNNKAGCGACTATGCCACTTGG-3′ |
| H11_AM_CDR3_F4 | 5′-CAAACGCGGGTCACGCGGNKNNKNNKNNKN |
| | NKTATGCCACTTGGCCTTAT-3′ |
| H11_AM_CDR3_F5 | 5′-CGGGTCACGCGGTCCCTCNNKNNKNNKNNK |
| | NNKACTTGGCCTTATGACTAC-3′ |
| H11_AM_CDR3_F6 | 5′-GTCACGCGGTCCCTCCTTNNKNNKNNKNNK |
| | NNKTGGCCTTATGACTACTGG-3′ |
| H11_AM_CDR_F7 | 5′-CGGTCCCTCCTTAGCGACNNKNNKNNKNNK |
| | NNKTATGACTACTGGGGCCAG-3′ |
| H11_Phd_F | 5′-GTTATTACTCGCGGCCCAGCCGGCCATGGC |
| | CCAGGTGCAGCTGGTGGAGTCTGGG-3′ |
| Reverse primers | |
| H11_AM_CDR3_R1 | 5′-TGCACAGTAATAAACGGC-3′ |
| H11_AM_CDR3_R2 | 5′-CGTTTGTGCACAGTAATAAAC-3′ |
| H11_AM_CDR3_R3 | 5′-GACCCGCGTTTGTGCACAG-3′ |
| H11_AM_CDR3_R4 | 5′-CCGCGTGACCCGCGTTTG-3′ |
| H11_AM_CDR3_R5 | 5′-GAGGGACCGCGTGACCCG-3′ |
| H11_AM_CDR3_R6 | 5′-AAGGAGGGACCGCGTGAC-3′ |
| H11_AM_CDR3_R7 | 5′-GTCGCTAAGGAGGGACCG-3′ |
| H11_Phd_F | 5′-GGTGATGGTGTTGGCCTCCCGGGCCTGAGG |
| | AGACGGTGACCTGGGTCCC-3′ |

TABLE 3b
PCR primers used for cloning
and affinity maturation

| Primer Name | Sequence |
|---|---|
| RBD_T332_F | 5′-CAGTACCGGTCACCATCACCATCACCATAC |
| | CAATCTGTGCCCATTCGGCGAG-3′ |
| RBD_T332_R | 5′-CAGTGGTACCTCATTTCTTGCCGCACACTG |
| | TGGCAGGAGCATG-3′ |
| CR3022_VH_F | 5′-GGTTGCGTAGCTGGTACCCAGATGCAGCTG |
| | GTGCAATC-3′ |
| CR3022_VH_R | 5′-GCCCTTGGTGGAGGCGACGGTGACCGTGGT |
| | CCCTTG-3′ |
| CR3022_VL_F | 5′-GGTTGCGTAGCTGGTACCGACATCCAGTTG |
| | ACCCAGTC-3′ |
| CR3022_VL_R | 5′-GTGCAGCCACCGTACGTTTGATTTCCACCT |
| | TGGTCCC-3′ |
| CR3022_Full_F | 5′-GCGTAGCTGAAACCGGCCAGATGCAGCTGG |
| | TGCAATC-3′ |
| CR3022_TVSS_R | 5′-GCCCTTGGTGGAGGCGCTAGAGACGGTGAC |
| | CGTGGTCCCTTG-3′ |
| CR3022_TVSS_F | 5′-CAAGGGACCACGGTCACCGTCTCTAGCGCC |
| | TCCACCAAGGGC-3′ |
| CR3022_linker_R | 5′-CGGTGGGCATGTGTGAGTTTTGTCACAAGA |
| | TTTGGGCTCAAC-3′ |
| CR3022_linker_F | 5′-GTTGAGCCCAAATCTTGTGACAAAACTCAC |
| | ACATGCCCACCG-3′ |
| CH3_R | 5′-GTGATGGTGATGTTTACCCGGAGACAGGGA |
| | GAGGCTCTTCTG-3′ |
| RBD_F | 5′-GCGTAGCTGAAACCGGCCCGAATATCACAA |
| | ATCTTTGTCC-3′ |
| RBD_His_R | 5′-GTGATGGTGATGTTTATTTGTACTTTTTTT |
| | CGGTCCGC-3′ |
| RBD_BAP_R | 5′-GTGATGGTGATGTTTTTCATGCCATTCAAT |
| | CTTTTGTGCCTCAAAAATATCATTCAAATTTGT |
| | ACTTTTTTTCGGTCCGC-3′ |
| RBD_Fc_R | 5′-CAGAACTTCCAGTTTATTTGTACTTTTTTT |
| | CGGTCCGC-3′ |
| ACE2_F | 5′-GCGTAGCTGAAACCGGCTCCACCATTGAGG |
| | AACAGGCC-3′ |
| ACE2_R | 5′-GTGATGGTGATGTTTGTCTGCATATGGACT |
| | CCAGTC-3′ |
| ACE2_Fc_R | 5′-CAGAACTTCCAGTTTGTCTGCATATGGACT |
| | CCAGTC-3′ |
| H11_AM_CDR3_F1 | 5′-GCCGTTTATTACTGTGCANNKNNKNNKNNK |
| | NNKCGGTCCCTCCTTAGCGAC-3′ |
| H11_AM_CDR3_R1 | 5′-TGCACAGTAATAAACGGC-3′ |
| H11_AM_CDR3_F2 | 5′-GTTTATTACTGTGCACAAACGNNKNNKNNK |
| | NNKNNKCTCCTTAGCGACTATGCC-3′ |
| H11_AM_CDR3_R2 | 5′-CGTTTGTGCACAGTAATAAAC-3′ |
| H11_AM_CDR3_F3 | 5′-CTGTGCACAAACGCGGGTCNNKNNKNNKNN |
| | KNNKAGCGACTATGCCACTTGG-3′ |
| H11_AM_CDR3_R3 | 5′-GACCCGCGTTTGTGCACAG-3′ |
| H11_AM_CDR3_F4 | 5′-CAAACGCGGGTCACGCGGNNKNNKNNKNNK |
| | NNKTATGCCACTTGGCCTTAT-3′ |
| H11_AM_CDR3_R4 | 5′-CCGCGTGACCCGCGTTTG-3′ |
| H11_AM_CDR3_F5 | 5′-CGGGTCACGCGGTCCCTCNNKNNKNNKNNK |
| | NNKACTTGGCCTTATGACTAC-3′ |
| H11_AM_CDR3_R5 | 5′-GAGGGACCGCGTGACCCG-3′ |
| H11_AM_CDR3_F6 | 5′-GTCACGCGGTCCCTCCTTNNKNNKNNKNNK |
| | NNKTGGCCTTATGACTACTGG-3′ |
| H11_AM_CDR3_R6 | 5′-AAGGAGGGACCGCGTGAC-3′ |
| H11_AM_CDR3_F7 | 5′-CGGTCCCTCCTTAGCGACNNKNNKNNKNNK |
| | NNKTATGACTACTGGGGCCAG-3′ |
| H11_AM_CDR3_R7 | 5′-GTCGCTAAGGAGGGACCG-3′ |
| H11_Phd_F | 5′-GTTATTACTCGCGGCCCAGCCGGCCATGGC |
| | CCAGGTGCAGCTGGTGGAGTCTGGG-3′ |
| H11_Phd_R | 5′-GGTGATGGTGTTGGCCTCCCGGGCCTGAGG |
| | AGACGGTGACCTGGGTCCC-3′ |
| OmA_exp_F | 5′-CTACCGTAGCGCAAGCTCAGGTGCAGCTGG |
| | TCGAGTCTGGGGGA-3′ |
| OmA_exp_R | 5′-GGTGATGGTGATGTTTTGAGGAGACGGTGA |
| | CCTGGGTCCCCTGGCC-3′ |
| H11-Fc_F | 5′-GCGTAGCTGAAACCGGCCAGGTGCAGCTGG |
| | TAGAGTC-3′ |
| H11-Fc_R | CAGAACTTCCAGTTTTGAGGAGACGGTGACCTG |
| | GG-3′ |

### Production of Single Domain Antibodies

The phagemids amplified from the selected clones were transformed into the WK6 E. coli strain and grown in TB medium (supplemented with 100 μg/mL ampicillin and 1 mM MgCl2), shaking at 225 rpm and 37° C., with induction of protein expression by 1 mM IPTG at OD˜1.2, and then grown overnight, shaking at 225 rpm and 20° C. The bacterial cells were pelleted and re-suspended in TES buffer (0.2 M Tris pH8, 0.5 mM EDTA, 0.5 M sucrose) overnight, followed by 2 hours in TES/4 buffer (TES diluted 4× in water). The supernatant was harvested through centrifugation at 16800 rpm and 4° C., and subsequently diluted 10× in GF buffer (20 mM Tris pH7.5 and 150 mM NaCl). The proteins were purified through an immobilised metal affinity using an automated protocol implemented on an ÄKTAxpress (GE Healthcare, UK) (Nettleship, Watson et al. 2015) followed by a Hiload 16/60 superdex 75 or a Superdex 75 10/300GL column, using phosphate-buffered saline (PBS) pH 7.4 buffer. To generate Fc fusions of the single domain antibodies, the sequences were inserted into pOPINTTG-3C-Fc and protein purified as described for the ACE-2 and RBD.

### VHH Library Screening

A VHH phage display library (Abcore Inc. Ramona, CA, USA) constructed in the vector pADL-20c and comprising approximately 1×10¹⁰ independent clones was inoculated into 2×TYA (2×TY supplemented with 100 μg/mL ampicillin) and infected with M13 helper phage to obtain a library of VHH-presenting phages. Phages displaying VHHs specific for the SARS-CoV-2 RBD were enriched after two rounds of bio-panning on 50 nM and 5 nM of RBD, respectively, through capturing with Dynabeads™ M-280 (Thermo Fisher Scientific). For each round of panning the Dynabeads and phages were firstly blocked with StartingBlock™ (PBS) Blocking Buffer (Thermo Fisher Scientific) for 30 minutes; the phages were incubated with the RBD for 1 hour, and then 5 minutes with the Dynabeads (Thermo Fisher Scientific); and subsequently washed 6 times with PBS supplemented with 0.05% Tween 20 and 1 time with PBS. The retained phages were eluted through incubation with TBSC buffer (10 mM Tris pH 7.4, 137 mM NaCl, 1 mM CaCl₂) and 1 mg/mL trypsin (Sigma-Aldrich) for 30 min. The collected phages were amplified in exponentially growing TG1 E. coli cells and plated on 2×TY agar plates supplemented with 100 μg/mL ampicillin. Enrichment after each round of panning was determined by plating the cell culture with 10-fold serial dilutions. After the second round of panning, 93 individual clones were picked to inoculate 2×TYA and were grown overnight, shaking at 250 rpm and 37° C. The next day, the overnight culture was used to inoculate 2×TYA and infected with M13 helper phage to obtain clonal VHH-presenting phages.

Enzyme-Linked Immunosorbent Assays

The wells of microtiter plates (Greiner high and medium binding) were coated with 5 μg/mL neutravidin in PBS pH 7.4 overnight at 4° C. The next day, the wells were coated with 50 nM biotinylated RBD, and then blocked with 3% milk powder in PBS pH 7.4. Supernatant of clonal phage was added into each well, binding was detected by incubating the wells with HRP-Conjugated anti-M13 (GE Healthcare). After washing, 100 μL of TMB substrate (SeraCare) was added and absorbance at 405 nM was measured with a Microplate Absorbance Reader.

### SPR and Isothermal Titration Calorimetry (ITC)

SPR experiments were performed using a Biacore T200 (GE Healthcare). All assays were performed using a Sensor Chip Protein A (GE Healthcare), with a running buffer of PBS pH 7.4 supplemented with 0.005% v/v Surfactant P20 (GE Healthcare) at 25° C.

To determine the binding affinity of nanobody H11 for the SARS-CoV-2 RBD, RBD-Fc was immobilized onto the sample flow cell of the sensor chip. The reference flow cell was left blank. Nanobody H11 was injected over the two flow cells at a range of 8 concentrations prepared by serial two-fold dilutions from 2.5 μM, at a flow rate of 30 μL/min, with an association time of 60 s and a dissociation time of 60 s. The data were fitted to a 1:1 binding model and to calculate K_{D} using GraphPad Prism 8.

To determine the binding kinetics between the SARS-CoV-2 RBD and nanobody H11-H4/H11-D4, RBD-Fc was immobilized onto the sample flow cell of the sensor chip. The reference flow cell was left blank. Nanobody H11-H4/H11-D4 was injected over the two flow cells at a range of five concentrations prepared by serial two-fold dilutions from 50 nM, at a flow rate of 30 μL/min using a single-cycle kinetics program with an association time of 60 s and a dissociation time of 60 s. Running buffer was also injected using the same program for background subtraction. All data were fitted to a 1:1 binding model using the Biacore T200

### Evaluation Software 3.1.

In the competition assay where CR3022-Fc or ACE2-Fc was used as the ligand, approximately 1000 RU of CR3022-Fc or ACE2-Fc was immobilized. The following samples were injected: (1) a mixture of 1 μM nanobody H11-H4/H11-D4 and 0.1 μM RBD; (2) a mixture of 1 μM E08R (anti-Caspr2 Fab) Fab and 0.1 μM RBD; (3) 0.1 pM RBD; (4) a mixture of 1 μM nanobody H11-H4/H11-D4 and 0.1 μM Spike; (5) a mixture of 1 pM E08R Fab and 0.1 μM Spike; (6) 0.1 μM Spike; (7) 1 μM nanobody H11-H4/H11-D4; (8) 1 μM E08R Fab. All injections were performed with an association time of 60 s and a dissociation time of 600 s. All curves were plotted using GraphPad Prism 8.

ITC measurements were carried out using an iTC200 MicroCalorimeter (GE Healthcare) at 25° C. Spike, RBD and nanobody were prepared and dialyzed in the same buffer, i.e., PBS. Nanobody was titrated into Spike or RBD solution corresponding to approximately 72 μM nanobody and 6 μM Spike or 250 μM nanobody and 25 μM RBD. Each experiment consisted of an initial injection of 0.4 μL followed by 16 injections of 2.4 μL nanobody solution into the cell containing either Spike or RBD, while stirring at 750 rpm. Data acquisition and analysis were performed using the Origin scientific graphing and analysis software package (OriginLab). Data analysis was performed by generating a binding isotherm and best fit using the following parameters: n (number of sites), ΔH (calories/mole), ΔS (calories/mole/degree), and K (binding constant in molar⁻¹). Following data analysis, K was converted to the dissociation constant (K_{D}) (nM).

### ACE2 Blocking and Neutralization Experiments

MDCK-SIAT1 cells were stably transfected with codon-optimized human ACE2 cDNA (NM_021804.1) using a second-generation lentiviral vector system and FACS sorted for highly expressing population. Cells (3×10⁴ per well) were seeded the day before the assay on a flat-bottomed 96-well plate. RBD-6H (amino acid 340-538; NITN.GPKK) was chemically biotinylated using EZ-link Sulfo-NHS-Biotin (A39256; Life Technologies). A serial half-log dilution (ranging 1 μM to 0.1 nM) of analytes and controls were performed in a U-bottomed 96 well plate in 30 μL volume. An equal volume of 25 nM of biotinylated RBD was added and 50 μL of each of the resulting mixtures were added to the MDCK-ACE2 cells for 1 hour. A second layer Streptavidin-HRP (S911, Life Technologies) diluted 1:1,600 in PBS/0.1% BSA (37525; Thermo Fisher Scientific) was then added and incubated for 1 hour. Plates were then washed with PBS 4 times and signal was developed by adding POD substrate (11484281001, Roche) for 5 min before stopping with 1 M H₂SO₄. Plates were read at OD₄₅₀ on a Clariostar plate reader. The control analyte (a non-blocking anti influenza N1 antibody) was used to obtain maximum signal and PBS only wells were used to determine background. Graphs were plotted as % binding of biotinylated RBD to ACE2. Binding %=(X−Min)/(Max−Min)*100 where X=Measurement of the competing component, Min=Buffer without binder biotinylated RBD-6H, Max=Biotinylated RBD-6H alone. Inhibitory concentration at 50% (IC₅₀) of the nanobodies against ACE2 was determined using non-linear regression [inhibitor] versus normalized response curve fit using GraphPad Prism 8.

MDCK-SIAT1 cells were stably transfected with RBD (amino acids 340-538 NITN.GPKK) fused to the transmembrane and cytoplasmic domain of haemagglutinin H7 (A/HongKong/125/2017) (EPI977395) via a short linker for surface expression (sequence TGSGGSGKLSSGYKDVILWFSFGASCFILLAIVMGLVFICVKNGNMRCTICI*) using a second-generation lentiviral vector system. RBD expressing cells were FACS sorted using the CR3022 antibody. Cells (3×10⁴ per well) were seeded the day before the assay on a flat-bottomed 96-well plate. ACE2-Fc was biotinylated as above. A serial half-log dilution (ranging 1 μM to 0.1 nM) of analytes and controls were performed in a U-bottomed 96 well plate in 30 μL volume. 30 μL of biotinylated Ace2-Fc at 5 nM was added to titrated analytes. Cells were washed with PBS and 50 μL of each mixture of ACE2 and an analyte was transferred to the cells and incubated for 1 h at room temperature. Cells were then washed with PBS and incubated for 1 h with the second layer Streptavidin-HRP (S911, Life Technologies) diluted to 1:1,600 and developed as above. Graphs were plotted as % binding of biotinylated ACE2 to RBD. Binding %=(X−Min)/(Max−Min)*100 where X=Measurement of the competing component, Min=Buffer without binder biotinylated ACE2-Fc, Max=Biotinylated ACE2-Fc alone. Inhibitory concentration at 50% (IC₅₀) of the nanobodies against ACE2 was determined using non-linear regression [inhibitor] versus normalized response curve fit using GraphPad Prism 8. Non-biotinylated ACE2-Fc-6H and VHH72-Fc were used as positive controls. Plaque reduction neutralization tests at Public Health England used SARS-CoV-2 (Australia/VIC01/2020)⁴³ which was diluted to a concentration of 933 pfu/mL (70 pfu/50 μL) and mixed 50:50 in minimal essential medium (MEM) (Life Technologies, California, USA) containing 1% fetal bovine serum (FBS) (Life Technologies) and 25 mM HEPES buffer (Sigma, Dorset, UK) with doubling antibody dilutions in a 96-well V-bottomed plate. The plate was incubated at 37° C. in a humidified box for 1 h to allow neutralization to take place. Afterwards the virus-antibody mixture was transferred into the wells of a twice Dulbecco's PBS-washed 24-well plate containing confluent monolayers of Vero E6 cells (ECACC 85020206; PHE, UK) that had been cultured in MEM containing 10% (v/v) FBS. Virus was allowed to adsorb onto cells at 37° C. for a further hour in a humidified box, then the cells were overlaid with MEM containing 1.5% carboxymethylcellulose (Sigma), 4% (v/v) FBS and 25mM HEPES buffer. After 5 days incubation at 37° C. in a humidified box, the plates were fixed overnight with 20% formalin/PBS (v/v), washed with tap water and then stained with 0.2% crystal violet solution (Sigma) and plaques were counted. A mid-point probit analysis (written in R programming language for statistical computing and graphics) was used to determine the dilution of antibody required to reduce SARS-CoV-2 viral plaques by 50% (ND₅₀) compared with the virus only control (n=5). The script used in R was based on a previously reported source script⁴⁴. Antibody dilutions were run in duplicate and an internal positive control for the PRNT assay was also run in duplicate using a sample of heat-inactivated (56° C. for 30 min) human MERS convalescent serum known to neutralize SARS-CoV-2 (National Institute for Biological Standards and Control, UK). The plates are shown in FIG. 8c.

Plaque reduction neutralization tests in Oxford were performed using passage 4 of SARS-CoV-2 Victoria/01/2020⁴³ using established methodology⁴⁵. In brief, virus stock (9.75×10⁴ pfu/mL) was diluted by 10 and by 100 in Dulbecco's Modification of Eagle's Medium containing 1% FBS (D1; 100 μL) was mixed with nanobody-Fc (100 μL) diluted in D1 so as give a final concentrations of H11-H4 at 100, 32, 10, 3.2 nM for measurement. As a positive control, solutions with CR3022 333, 167, 84 and 42 at nM were prepared. Each experiment was performed in triplicate in 24 well tissue culture plate. The plate was incubated at room temperature for 30 minutes and 0.5 mL of a single cell suspension of Vero E6 cells in D1 at 5×10⁵/mL was added. The plates were incubated for a further 2 h at 37° C. before being overlain with 0.5 mL of D1 supplemented with carboxymethyl cellulose (1.5%). The resulting cultures were incubated for a further 4 days at 37° C. before plaques were revealed by staining the cell monolayers with amido black in acetic acid/methanol (FIG. 9a, b, c, d). To probe whether CR3022 and H11-H4 were additive, solutions of H11-H4 at 100, 32, 10, 3.2 nM were each incubated for 30 mins with CR3022 at a final concentration of 84 nM. The resulting mixtures were analyzed as described above in triplicate experiments and the wells are shown in FIG. 9d.

Cell Lines

Oxford neutralisation used Vero Ccl-81 (from a stock that was originally from ATCC). PHE neutralisation used VeroE6 Cells purchased from ECACC. Cell based competition assays used MDCK-SIAT1 cells derived from a commercial source (Sigma-Aldrich). All mammalian protein expressions were performed with purchased 293Expi cells (ThermoFisher Scientific) and E. coli cells.

### Nanobody Complex with Spike, Preparation and Cryo-EM Data Collection

Purified spike protein in 10 mM Hepes, pH 8, 150 mM NaCl, was incubated with H11-H4 purified in 50 mM Tris, pH 7, 150 mM NaCl, at a molar ratio of 1:3.6 (Spike trimer:nanobody) at 16° C. overnight. Spike protein was used at a final concentration of 1 mg/mL. The mixture was centrifuged at 21000 g, 16° C. prior to grid preparation. For H11-D4—Spike a mixture in the molar ratio of 1:6 (Spike trimer:nanobody) was incubated at 20° C. for ten minutes. 3 μL of the resulting H11-D4—Spike sample was then applied to a holey carbon-coated 200 mesh copper grid (C-Flat, CF-2/1, Protochips) that had been freshly glow-discharged on high for 20 s (Plasma Cleaner PDC-002-CE, Harrick Plasma). Excess liquid was removed by blotting for 6 s with a blotting force of −1 using vitrobot filter paper (grade 595, Ted Pella Inc.) at 4.5° C., 100% relative humidity. Blotted grids were then immediately plunge-frozen using a Vitrobot Mark IV (Thermo Fisher Scientific).

Frozen grids were first screened on a Glacios microscope operating at 200 kV (Thermo Fisher Scientific) before imaging on a Titan Krios G2 (Thermo Fisher Scientific) at 300 kV. Movies (40 frames each) were collected in compressed tiff format on a K3 detector (Gatan) in super resolution counting mode using a custom EPU version 2.5 (Thermo Fisher Scientific) (Table 4).

TABLE 4
Cryo-EM data collection, refinement and validation statistics

| | Spike-H11-D4 | Spike-H11-H4 |
|---|---|---|
| | (EMD-11068 | (EMD-11218, |
| | PDB 6Z43) | PDB 6ZHD) |
| Data collection and processing | | |
| Magnification | 105,000 | 81,000 |
| Voltage (kV) | 300 | 300 |
| Electron exposure (e⁻/Å²) | 43 | 46 |
| Defocus range (μm) | 0.8-2.6 | 1.0-3.0 |
| Pixel size (Å/pix) (Super resolution) | 0.415 | 0.53 |
| Symmetry imposed | C1 | C1 |
| Initial particle images (no.) | 596,825 | 786,392 |
| Final particle images (no.) | 305,513 | 126,938 |
| Map resolution (Å) | 3.3 | 3.7 |
| FSC threshold | 0.143 | 0.143 |
| Map resolution range (Å) | 3.2-9.7 | 3.7-7.0 |
| Refinement^{a} | | |
| Initial model used | PDB 6VXX | PDB 6Z43 |
| Model resolution (Å) | 3.4 | 3.7 |
| FSC threshold | 0.143 | 0.143 |
| Model resolution range (Å) | 3.3-6.0 | 3.7-6.0 |
| Map sharpening B factor (Å²) | −117 | −114 |
| Model composition | | |
| Non-hydrogen atoms | 26,725 | 26,960 |
| Protein residues | 3,351 | 3,419 |
| B factors (Å²) | | |
| Protein | 119 | 183 |
| R.m.s. deviations | | |
| Bond lengths (Å) | 0.005 | 0.006 |
| Bond angles (°) | 0.58 | 1.28 |
| Validation | | |
| MolProbity score | 1.43 | 1.53 |
| Clashscore | 8.7 | 5.5 |
| Poor rotamers (%) | 0.1 | 0.9 |
| Ramachandran plot | | |
| Favored (%) | 94.8 | 96.4 |
| Allowed (%) | 5.1 | 3.6 |
| Disallowed (%) | 0.1 | 0.0 |

^{a}Nanobody excluded from refinement.

Motion correction and alignment of 2× binned super-resolution movies was performed using Relion (v3.1)⁴⁶ with a 5×5 patch-based alignment. CTF-estimation of full-frame non-weighted micrographs was performed using GCTF (v1.06) and non-template-driven particle picking was then performed within cryoSPARC (v2.14.1-live)47 followed by multiple rounds of 2D classification. The resulting 2D class averages consistent with Spike trimer were used for template-driven particle picking before further rounds of 2D and 3D classification with C1 symmetry. The resulting map from the most populous class was then sharpened in cryoSPARC before conversion to Relion-format star files using custom pyEM scripts⁴⁸ (csparc2star.py, https://github.com/asarnow/pyem) for further CTF refinement within Relion.

An initial model for Spike was generated using PDB 6VXX²⁶ and rigid body fitted into the map using Chimera⁴⁹ followed by Coot⁵⁰. The H11-D4-RBD crystal structure was superimposed onto the naked Spike model in Coot and checked for fit in the density. S1/S2 domains split into subdomains for each subunit (residues 27-307; 308-321 and 591-700; 322-333 and 529-590; 701-1147) were then independently rigid body fitted in Coot^{50.} before a final real space refinement with PHENIX⁵¹ with hydrogen atoms added using ReadySet⁵¹ resulting in a final correlation coefficient of 0.8. The H11-D4-RBD crystal structure was used as reference structure restraints during refinement of the Spike owing to the density. Rounds of manual inspection in Coot⁵⁰ and real space refinement with PHENIX⁵¹ resulted in the final model. Data processing and refinement statistics are shown in Table 4.

For H11-H4 Spike sample, SPT Labtech prototype 300 mesh 1.2/2.0 nanowire grids with a highly reproduceable rectangular bar cross-section were used. The grids were glow-discharged on low for 90 s (Plasma Cleaner PDC-002-CE, Harrick Plasma) to activate the nanowires. Approximately 6 nL of the complex were applied to the grids using a Chameleon EP system (SPT Labtech) at 81% relative humidity and ambient temperature.

Frozen grids were screened and then data collected using Titan Krios G2 (Thermo Fisher Scientific) equipped with a Bioquatum-K3 detector (Gatan, UK) operated at 300 kV. Movies (50 frames each) were collected in compressed tiff format in super-resolution counting mode using a custom EPU version 2.5 (Thermo Fisher Scientific) .

Processing of movies up to 2D classification was done automatically using the Relion_IT.py processing pipeline implemented at eBIC. In detail, motion correction and alignment of 2× binned super-resolution movies was performed using Relion (v3.08)⁴⁶ with a 5×5 patch-based alignment. CTF-estimation of full-frame non-weighted micrographs was performed using GCTF (v1.06) and non-template-driven particle picking was then performed within crYOLO⁵² followed by 2D classification. The best 2D classes clearly showing details consistent with the spike complex were selected for further processing. 3D classification was done using emd_21374 low pass filtered to 60Å. Initially the data was processed as C3 but was relaxed to C1 as the RBD and nanobody density were poor. The best C1 3D class was selected for further refinement, CTF refinement, and particle polishing within Relion.

The coordinates from the Spike-H11-D4 structure were rigid-body docked into the Spike-H11-H4 cryo-EM density in Chimera⁴⁹ and then refined with multiple rounds of jelly body refinement using RefMac5 via CCP-EM GUI^{53,54} and manual intervention with coot resulted in a final correlation coefficient of 0.78. Due to the limited resolution of the nanobody density in the cryo-EM map, the refined nanobody structure was replaced by the docked H11-H4-RBD crystal structure in the final model. Finally, the nanobodies were docked as rigid bodies into the cryo-EM density using Chimera⁴⁹ to optimize their position. Data processing and refinement statistics are shown in Table 4.

### H11-D4-RBD-CR3022 and H11-H4-RBD-CR3022 crystallography

Purified RBD, Fab CR3022 and H11-D4 were mixed together at a molar ratio of 1:1:1 to a final concentration of approximately 7 mg/mL and incubated at room temperature for one hour. Initial screening was performed in 96-well plates using the nanoliter sitting-drop vapor diffusion method. The best crystals were grown in condition containing 0.1 M sodium citrate tribasic dihydrate, pH 5.0, 10% (w/v) polyethylene glycol 6000.

Purified RBD, Fab CR3022 and nanobody H11-H4 were mixed together at a molar ratio of 1:1:.11, incubated at room temperature for one hour and run on a gel filtration column. Initial screening was performed in 96-well plates using the nanoliter sitting-drop vapor diffusion method. The best crystals were grown by mixing 0.1 μL of the 20 mg/mL of the H11-H4RBD-CR3022 complex with 0.1 μL of the crystallization buffer as above. Crystals were soaked in cryoprotectant containing 70-75% reservoir solution and 20-25% glycerol for a few seconds, then mounted in loops and frozen in liquid nitrogen prior to data collection at beamline I03 of Diamond Light Source, UK.

Two crystal forms for H11-D4-RBD-CR3022 were obtained (Table 5). For the first form collected, 3 crystals, 360° each, were merged to give a final data set to 3.3 A resolution with 78-fold redundancy. A second form appeared later and yielded 2.7Å from a single crystal although the data were anisotropic. A single crystal of H11-H4-RBD-CR3022 was collected.

TABLE 5
X-ray crystallography data collection and refinement statistics

| | | | H11-H4- | H11-D4-RBD-CR3022 | |
|---|---|---|---|---|---|
| | H11-D4-RBD | H11-H4-RBD | RBD-CR3022 | (PDB | (PDB |
| | (PDB 6YZ5) | (PDB 6YBP) | (PDB 6ZH9) | 6Z2M) | 6YZ7) |
| Data collection | | | | | |
| Space group | P3₁21 | P3₁21 | P 4₂2₁2 | P2₁2₁2 | P 4₁22 |
| Cell dimensions | | | | | |
| a, b, c (Å) | 78.3, 78.3, | 73.2, 73.2, | 156.4, 154.4, | 149.7, 150.4, | 154.6, 154.6, |
| | 127.1 | 131.7 | 116.3 | 119.5 | 229.3 |
| α, β, γ (°) | 90, 90, 120 | 90, 90, 120 | 90, 90, 90 | 90, 90, 90 | 90, 90, 90 |
| Resolution (Å)^{a} | 46-1.80 | 57-1.85 | 60-3.31 | 105-2.72 | 128-3.29 |
| | (1.85-1.80) | (1.90-1.85) | (3.37-3.31) | (2.96-2.72) | (3.35-3.29) |
| R_{merge} | 0.109 (1.7) | 0.086 (2.05) | 0.091 (3.9) | 0.168 (1.6) | 0.82 (—) |
| Rₚᵢₘ | 0.036 (0.54) | 0.028 (0.46) | 0.018 (0.77) | 0.047 (0.46) | 0.094 (4.1) |
| I/σ (I) | 20.6 (1.9) | 18.2 (1.7) | 21.9 (0.5) | 11.2 (1.6) | 4.9 (0.2) |
| CC_{1/2} | 1.0 (0.7) | 1.0 (0.7) | 1.0 (0.3) | 1.0 (0.5) | 1.0 (0.4) |
| Completeness (%) | 99.7 (99.7) | 99.8 (99.5) | 100 (100) | 65 (14) | 100 (93) |
| Completeness (%) | | | | 95.6 (82.1) | |
| (ellipsoidal)^{b} | | | | | |
| Redundancy | 19.7 (20.1) | 19.7 (20.9) | 25.6 (26.5) | 13.7 (12.4) | 78.4 (77.9) |
| Refinement | | | | | |
| Resolution (Å) | 46.3-1.80 | 57.2-1.85 | 128-3.31 | 78-2.71 | 128-3.29 |
| | (1.85-1.80) | (1.90-1.85) | (3.4-3.31) | (2.96-2.71) | (3.4-3.29) |
| No. reflections | 40120 (3066) | 35506 (2585) | 20977(1538) | 47412 (2371) | 39015(1538) |
| R_{work}/R_{free} | 16.6/19.3 | 18.5/21.7 | 26.3/30.5 | 19.8/24.1 | 23.7/26.8 |
| | (30.5/29.2) | (30.0/33.3) | (40.5/41.6) | (28.8/29.9) | (41.5/37.9) |
| No. atoms | | | | | |
| Protein | 2571 | 2591 | 5906 | 11731 | 11718 |
| Ions/buffer | 64 | 19 | — | — | — |
| Water | 241 | 103 | — | — | — |
| Residual B factors | | | | | |
| Protein | 38 | 28 | 118 | 65 | 157 |
| Ligand/ion | 55 | 71 | — | — | — |
| Water | 45 | 53 | — | — | — |
| R.m.s. deviations | | | | | |
| Bond lengths (Å) | 0.010 | 0.012 | 0.008 | 0.004 | 0.004 |
| Bond angles (°) | 1.4 | 1.68 | 1.63 | 0.74 | 1.39 |

Data were collected from a single crystal for each structure.
^{a}Values in parentheses are for highest-resolution shell.
^{b}These data showed significant anisotropy and were truncated accordingly.

Data were indexed, integrated and scaled with the automated data processing program Xia2-dials^{55,58}. The crystal structure of the first crystal of the H11-D4-RBD-CR3022 complex was solved by molecular replacement using (PDB 6YLA¹⁸) and the nanobody 9G8 (PDB 4KRP⁵⁷). The high-resolution structures of the H11-D4-RBD and H11-H4-RBD complexes then became available and were used in subsequent solutions. The electron density H11-H4-RBD-CR3022 was, as seen in the low resolution H11-D4-RBD-CR3022 structure, poor for the nanobody; a reflection of the relatively low resolution of the study.

Model rebuilding was done with COOT⁵⁰, initially refined with PHENIX⁵¹ then with REFMAC⁵⁵⁸ aided by PDB-REDO⁵⁹, MOLPROBITY⁶⁰ and the TLSMD server⁶¹.

### H11-H4-RBD and H11-D4-RBD Crystallography

Each nanobody was mixed with 8.7 mg of RBD at 2.9 mg/mL at a molar ratio nanobody: RBD 1.1:1 and the complex was incubated for 3 h in a cold room under agitation at 2 rpm. RBD in the complex was deglycosylated by the addition of 0.4 mg of EndoH glycosidase and incubated overnight at room temperature, under agitation at 2 rpm. The mixture was then concentrated to 1 mL with a 5 kDa MWCO concentrator and injected on gel filtration using a Superdex 200 10/300 (GE) in 50 mM Tris pH 7, 150 mM NaCl. The peak fractions were pooled and concentrated using 5 kDa MWCO concentrator to 10 mg/mL, 18 mg/mL and 29 mg/mL. Crystallization screening was performed on the Diamond/RCaH/RFI HTP crystallization facility at Harwell. Crystals of H11-D4-RBD were grown at 20° C. using the sitting drop vapor diffusion method by mixing 0.2 μL of the 18 mg/mL complex with 0.1 μL of the crystallization buffer containing 0.2 M Sodium acetate trihydrate, 0.1 M MES pH 6.0, 20% w/v PEG 8000. H11-D4-RBD crystals grew overnight and were flash cooled in a solution containing the mother liquor with 30% (v/v) ethylene glycol. Crystals of H11-H4-RBD were grown at 20° C. using the sitting drop vapor diffusion method by mixing 18 mg/mL complex with 0.1 μL of the crystallization buffer containing 0.2 M Lithium sulphate, 0.1 M Bis Tris pH 5.5, 25% w/v PEG 3350. H11-H4-RBD crystals grew overnight and were flash cooled in a solution containing the mother liquor with 30% (v/v) PEG 400. Diffraction data were also collected and processed at beamline I03 at Diamond Light Source (DLS). The H11-D-RBD structure was solved by molecular replacement⁶² using the RBD and H11-D4 monomers from the ternary complex above. Refinement was carried out as described above for the ternary complex. The H11-H4-RBD complex was solved using the H11-D4-RBD complex. Statistics for X-ray data collection and structure refinement are given in Table 5. Electron density for both complexes is show in FIG. 13c,d.

### Structural Analysis of H11-D4 RBD Complex

The high resolution of the H11-H4-RBD and H11-D4-RBD complexes revealed subtle differences between them. The main article focuses on the H11-H4-RBD, this note analyses H11-D4-RBD. In the H11-D4-RBD complex (FIG. 15A), CDR1 contributes very little to the interface (FIG. 15A). In CDR2, residues Arg52, Ser54 and Ser57 are in contact with RBD (FIG. 15A and 15B). From CDR3, Glu100—Leu106 make contacts with RBD (FIG. 15B). All contacts between the two proteins are shown in FIG. 15C. The surface on RBD that contacts H11-D4 is formed by Lys444-Phe456 and Gly482-Ser494 (FIG. 15D). These two stretches of RBD sequence comprise over 90% of buried surface area and make all the hydrogen bonds with H11-D4 (FIG. 15C).

The aromatic ring of Tyr449 in RBD stacks against a hydrophobic patch on H11-D4 at Asn101 (FIG. 16A). We note the key role of Arg52 from CDR2 of H11-D4, which sits at the heart of a network of interactions, including RBD residues Glu484 (bivalent salt bridge) and Phe490 (p-cation interaction28) (FIG. 16B). Arg52 seems also to play a role in stabilizing the conformation of the CDR3 loop of H11, as it forms hydrogen bonds to the backbone carbonyls of Arg103, Ser104 and side chain of Tyr109 (FIG. 16B). The seven-residue stretch of H11-D4 CDR3 region, which varied during maturation, contributes over 60% of the surface area buried by the complex and makes five hydrogen bonds to RBD (FIG. 16C). Arg98 is the only maturation change in the CDR3 loop that does not contact RBD (FIG. 16C). Rather, this residue salt bridges to Glu 100 and makes hydrogen bonds to several residues in CDR1, suggesting it is important for ordering the CDR3 and CDR1 loops. Trp112 of H11-D4 adopts two conformations both of which stack against the Arg103-Asp108 salt bridge (FIG. 16D), an interaction that also appears important to the structure of the CDR3 region.

### RESULTS

### Identification of a Spike-Binding Nanobody

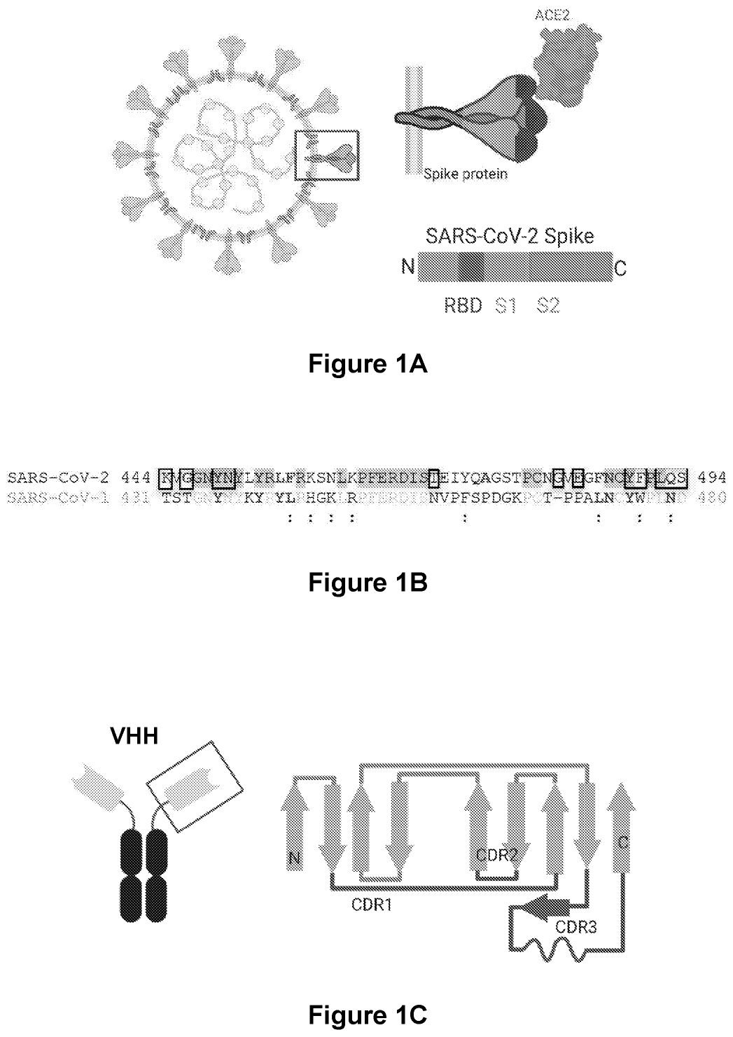

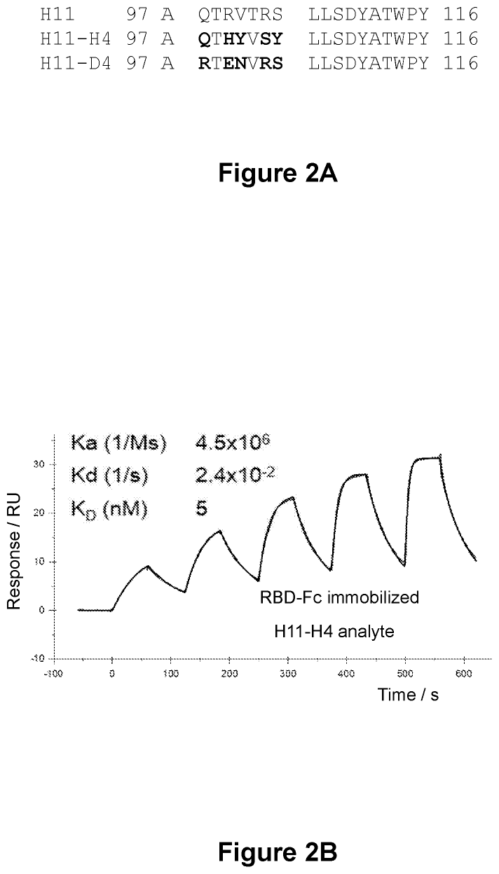

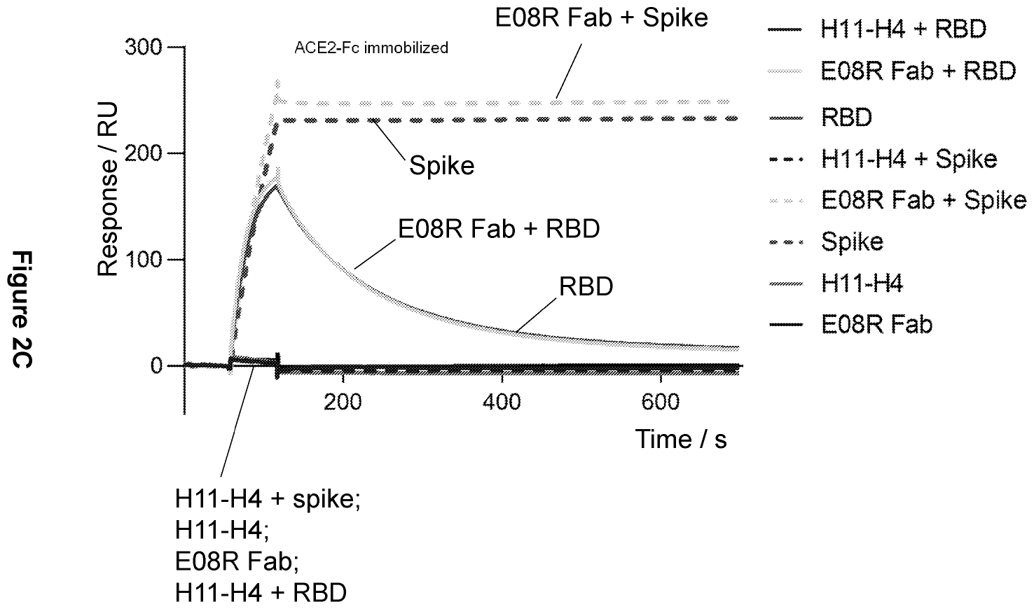

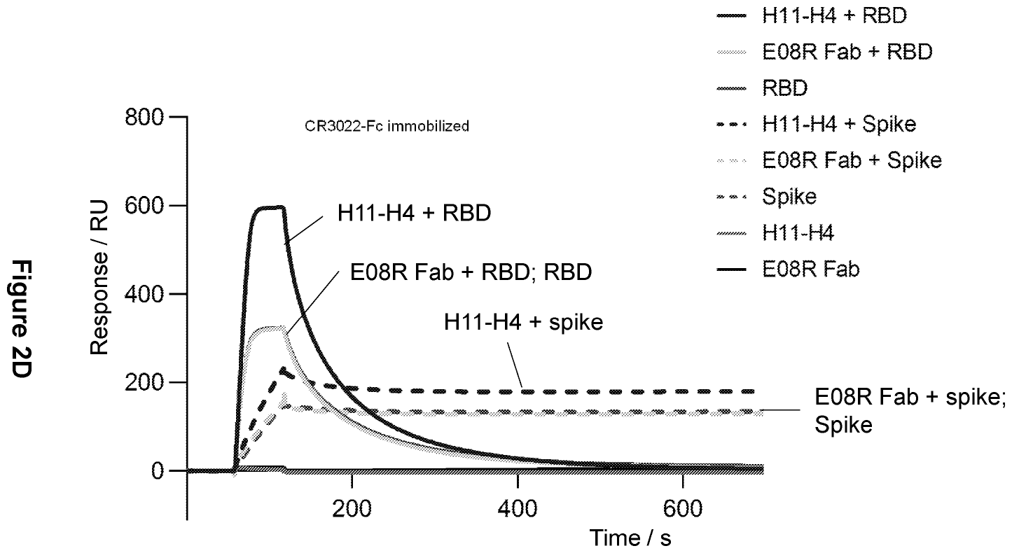

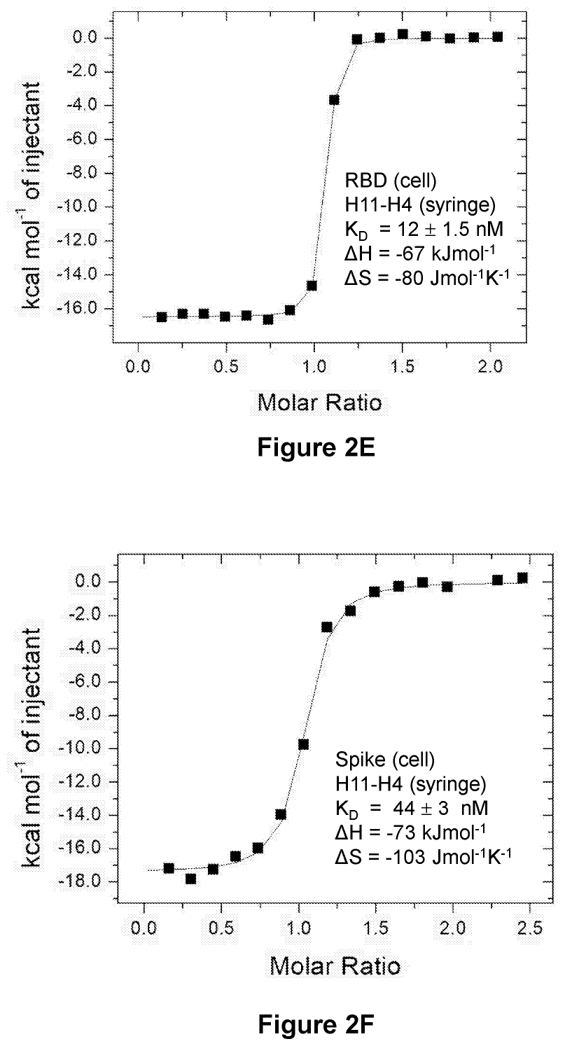

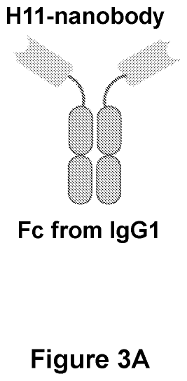

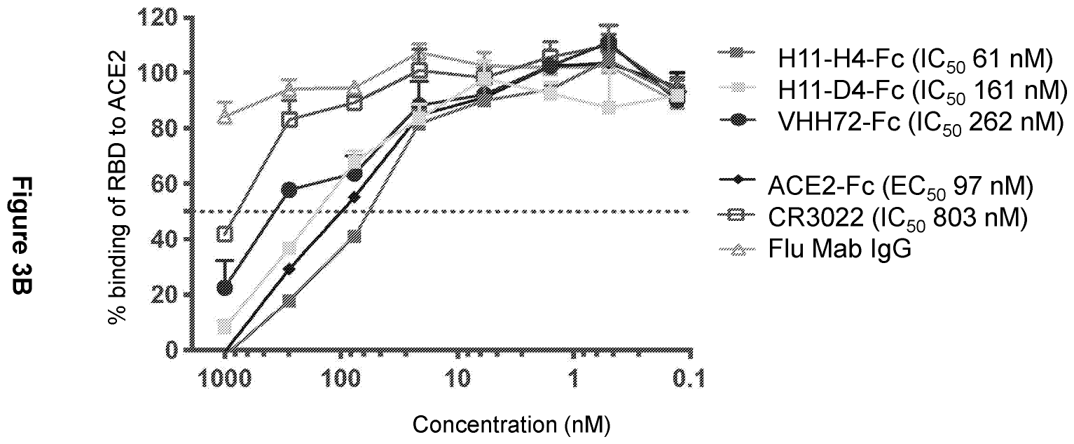

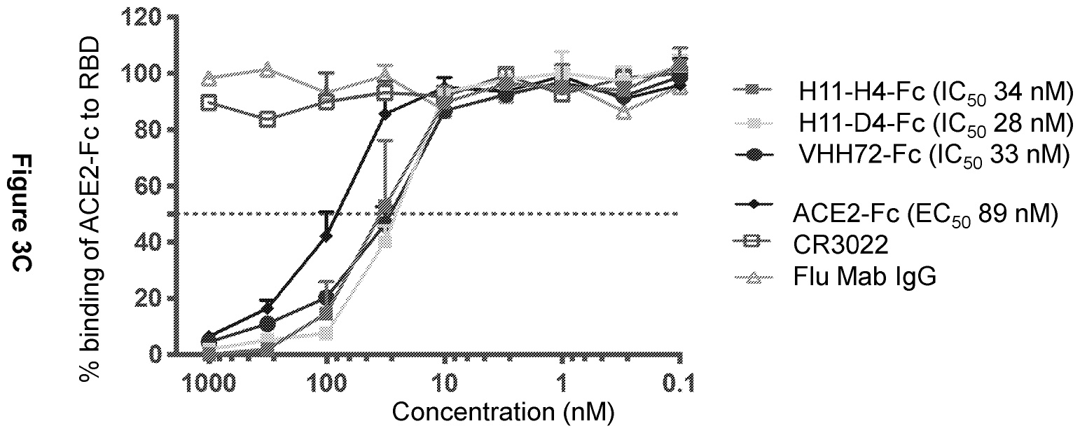

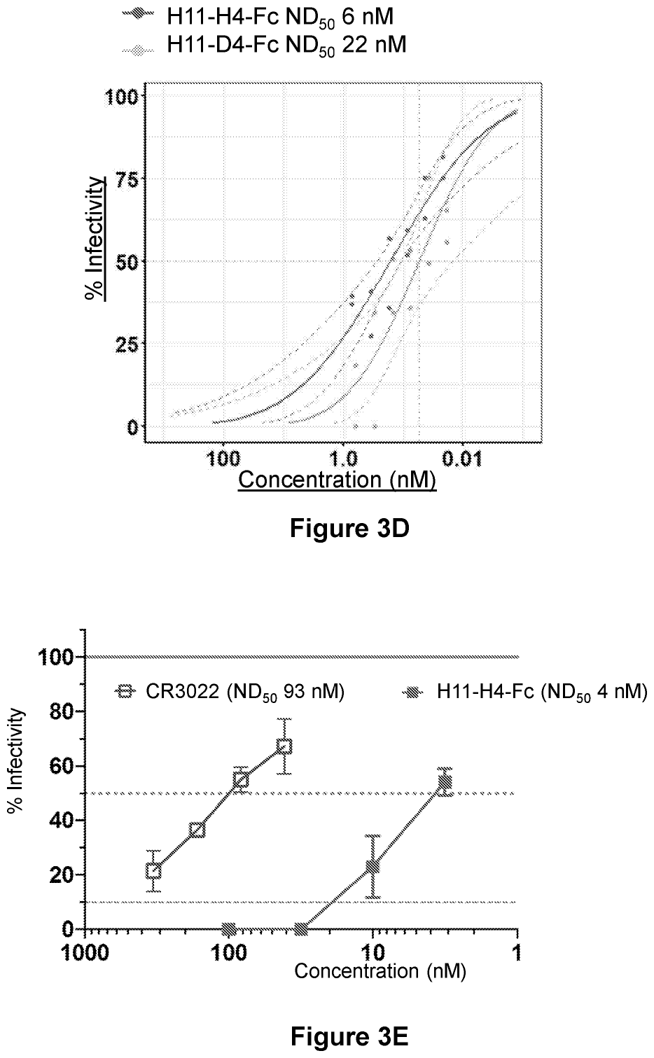

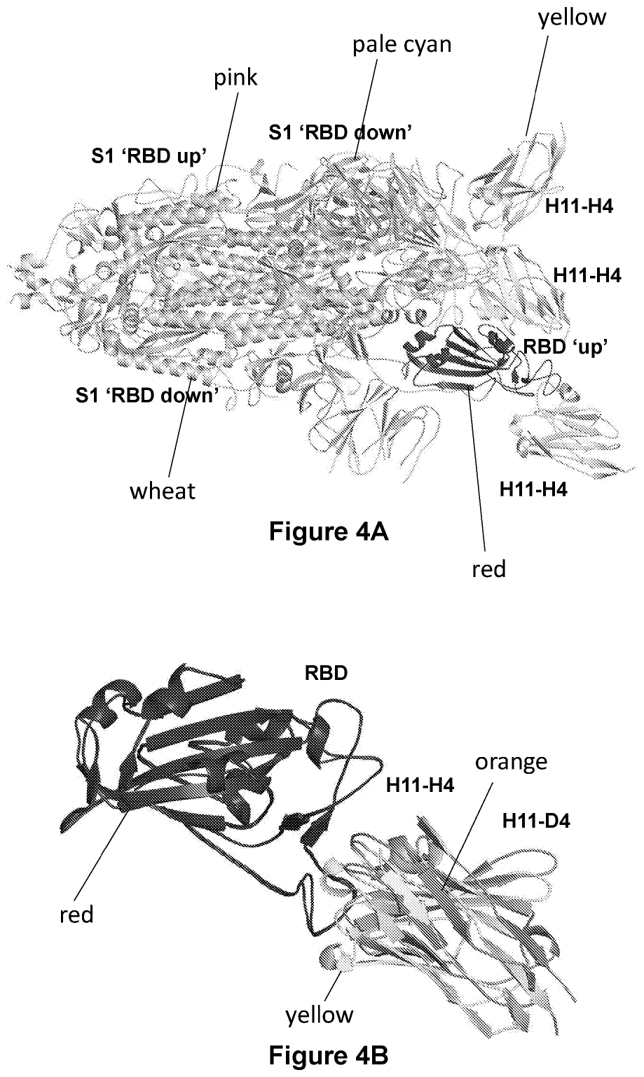

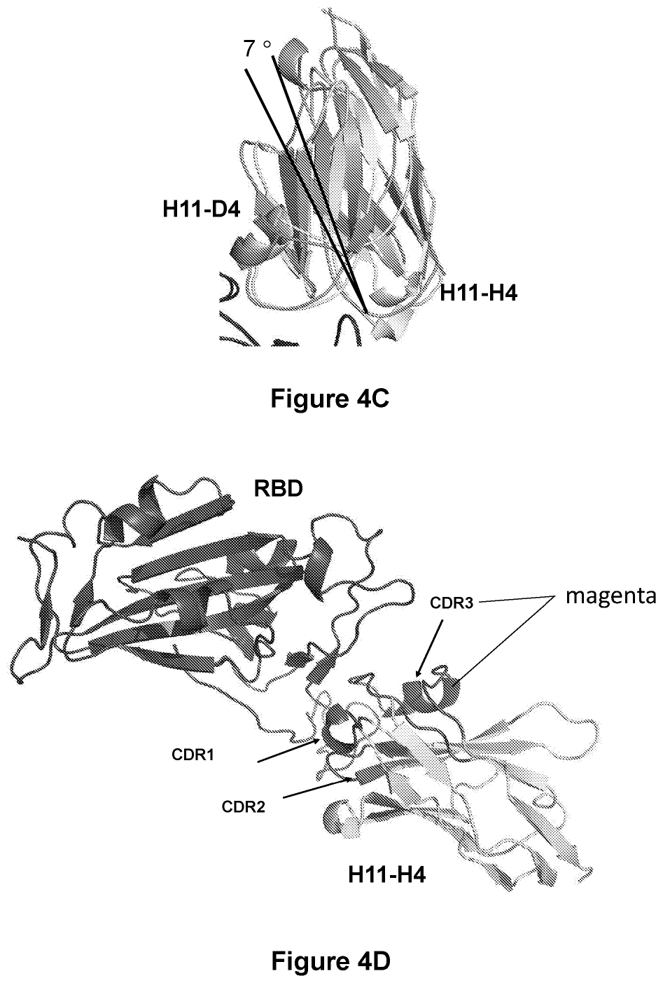

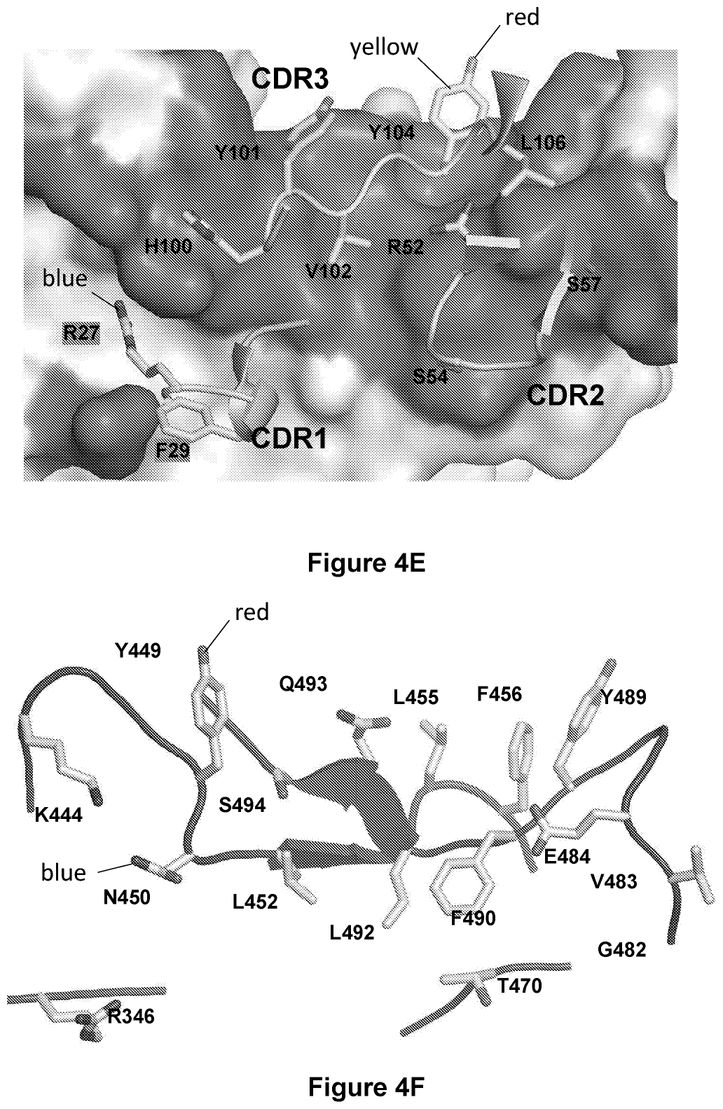

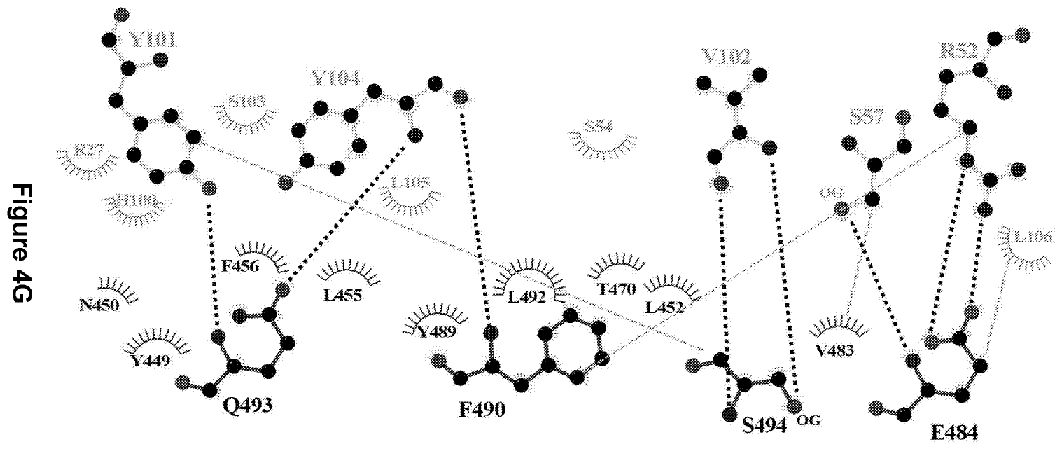

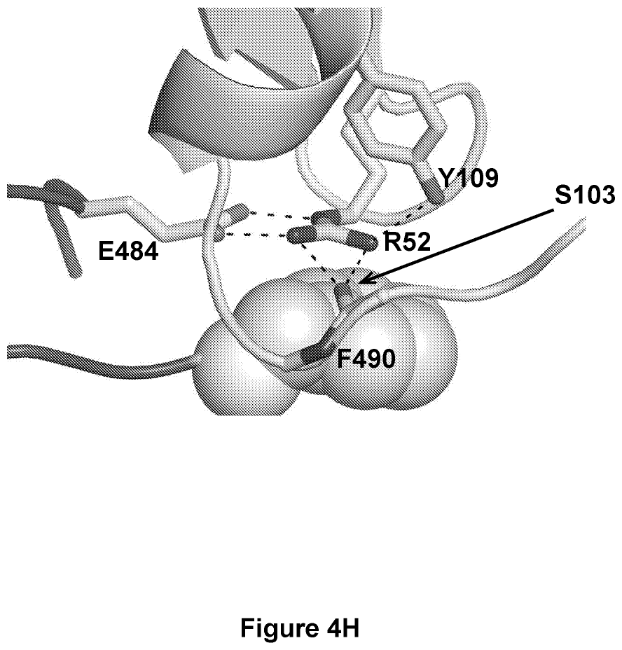

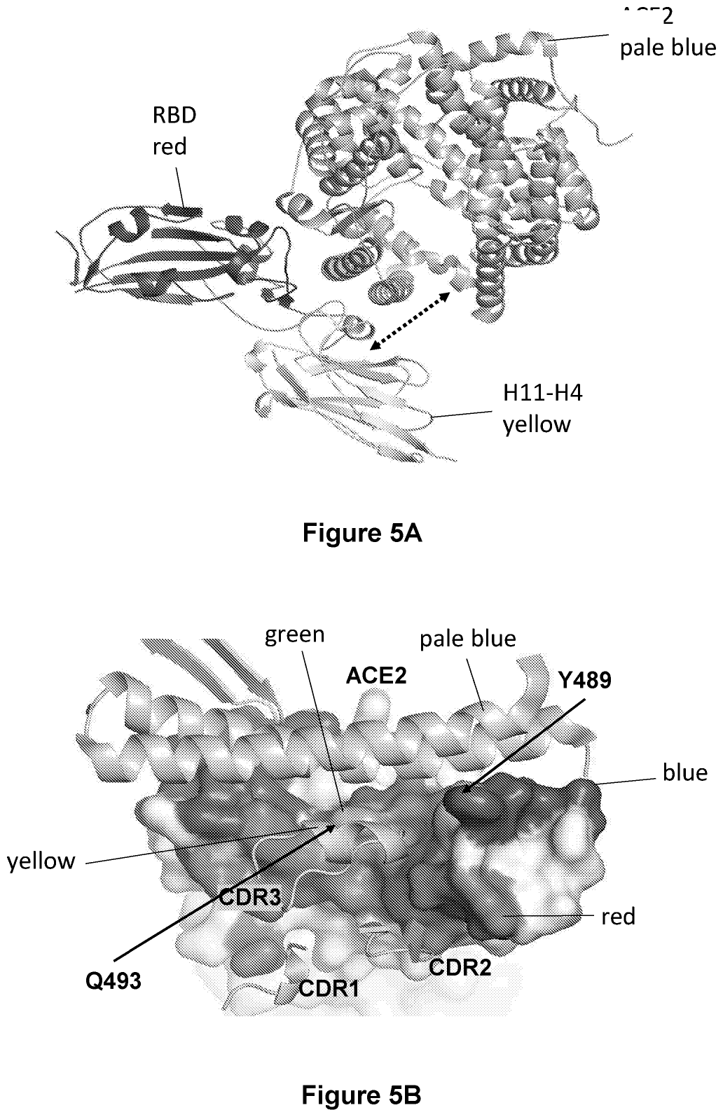

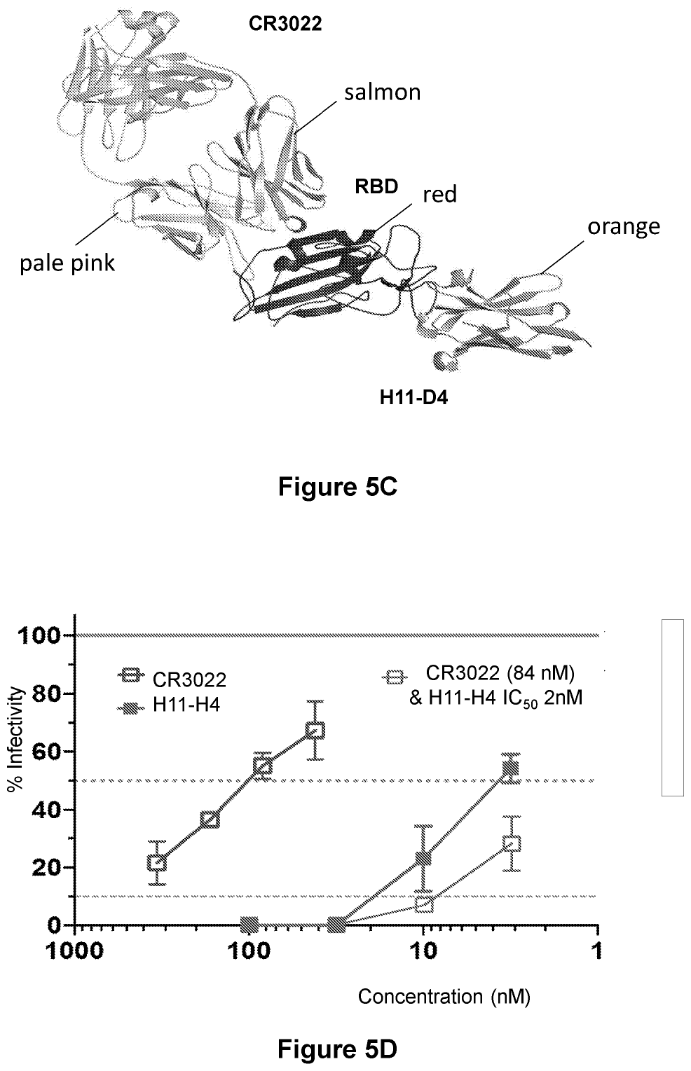

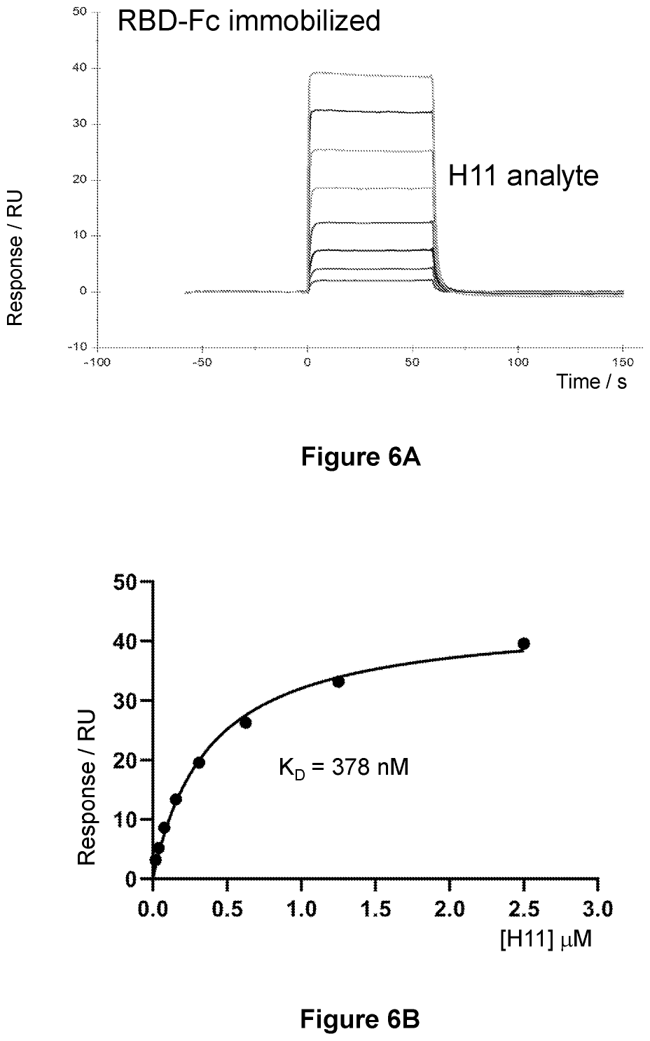

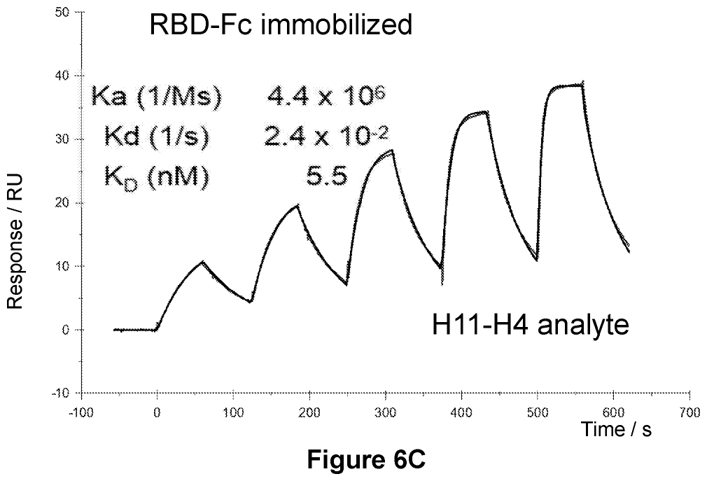

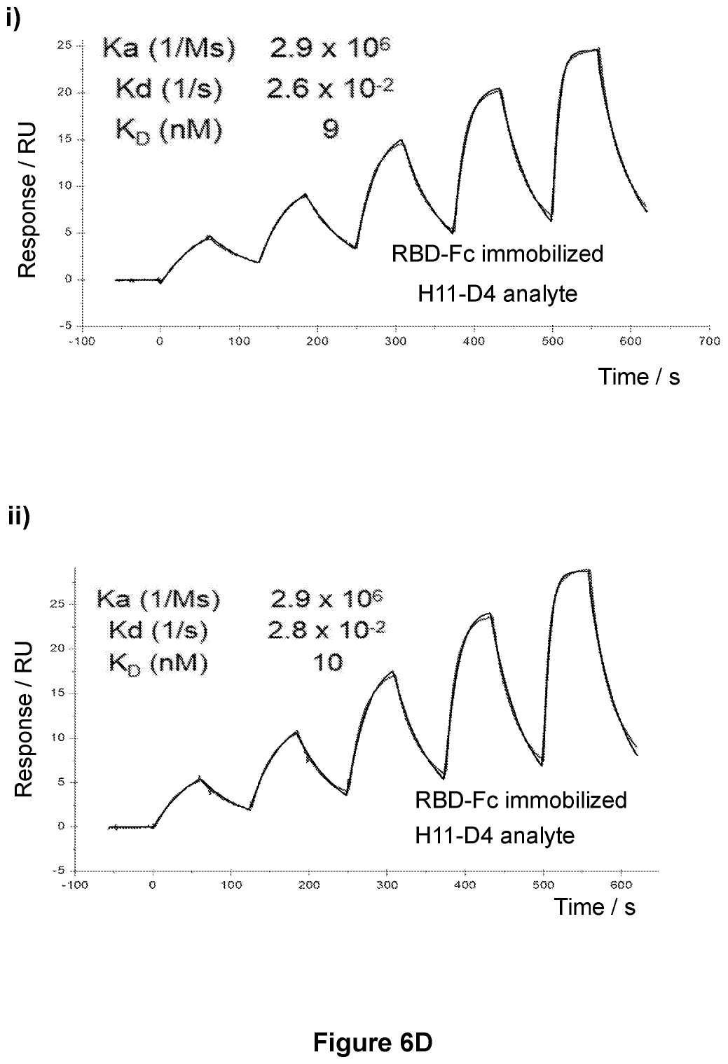

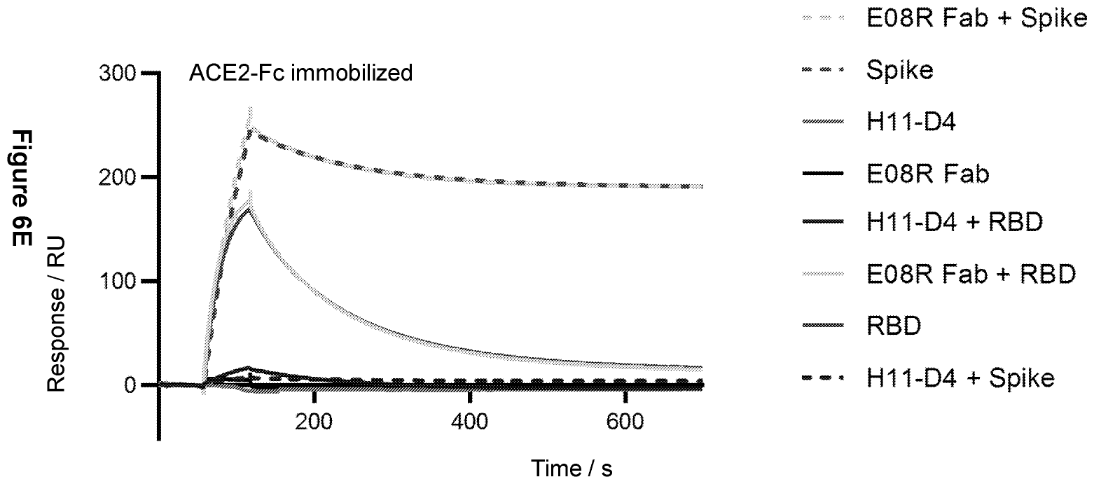

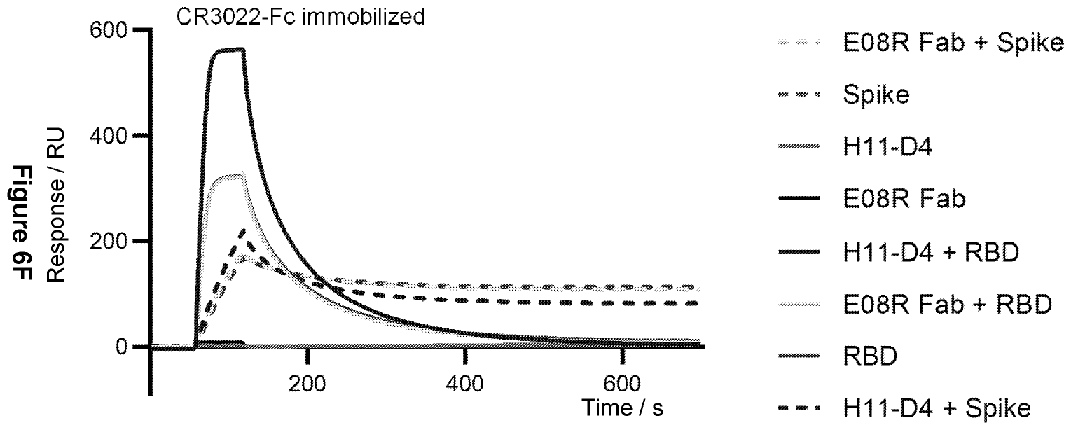

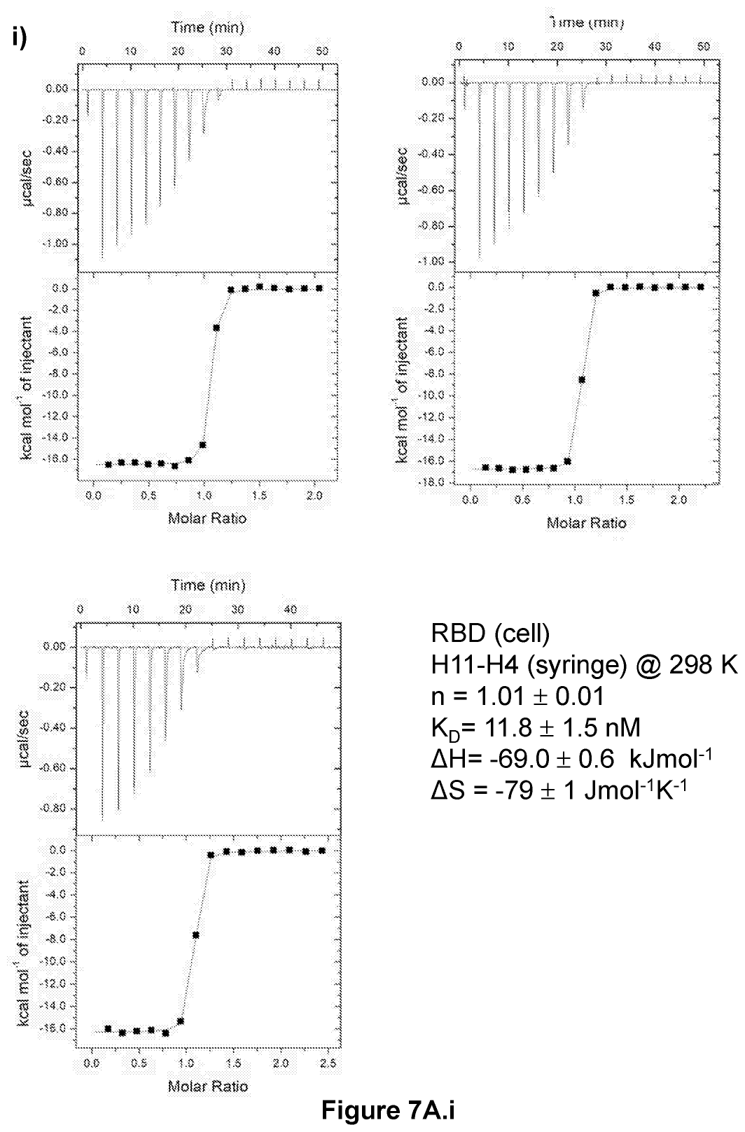

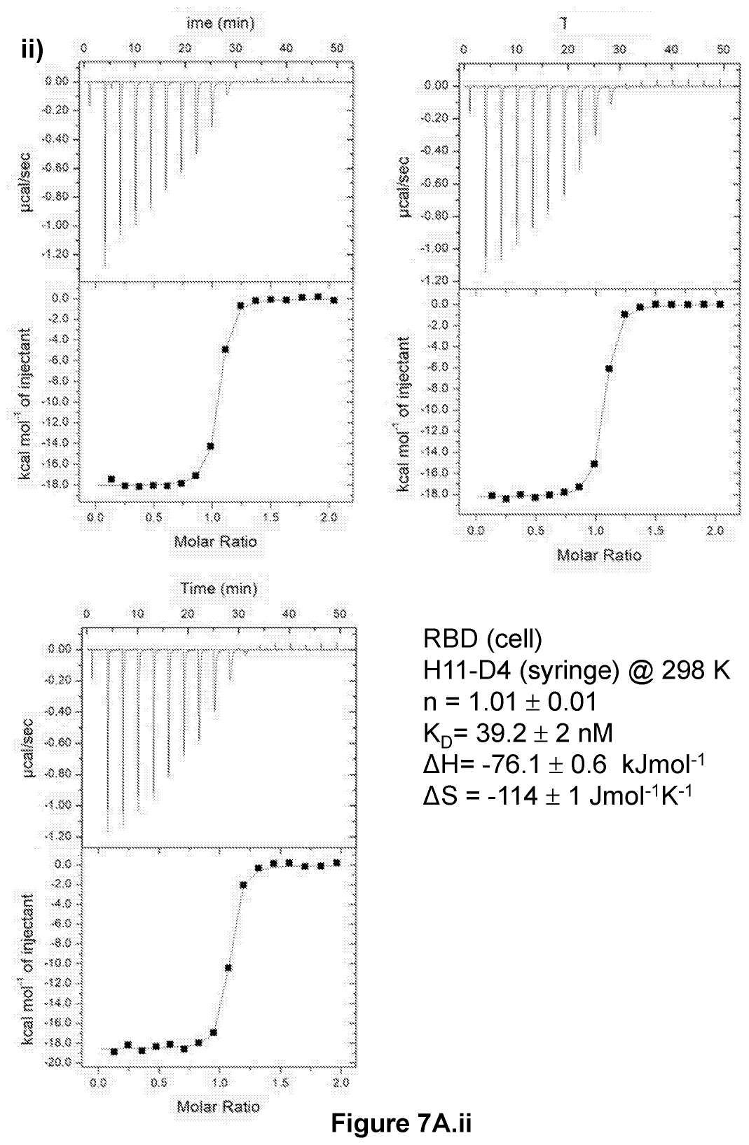

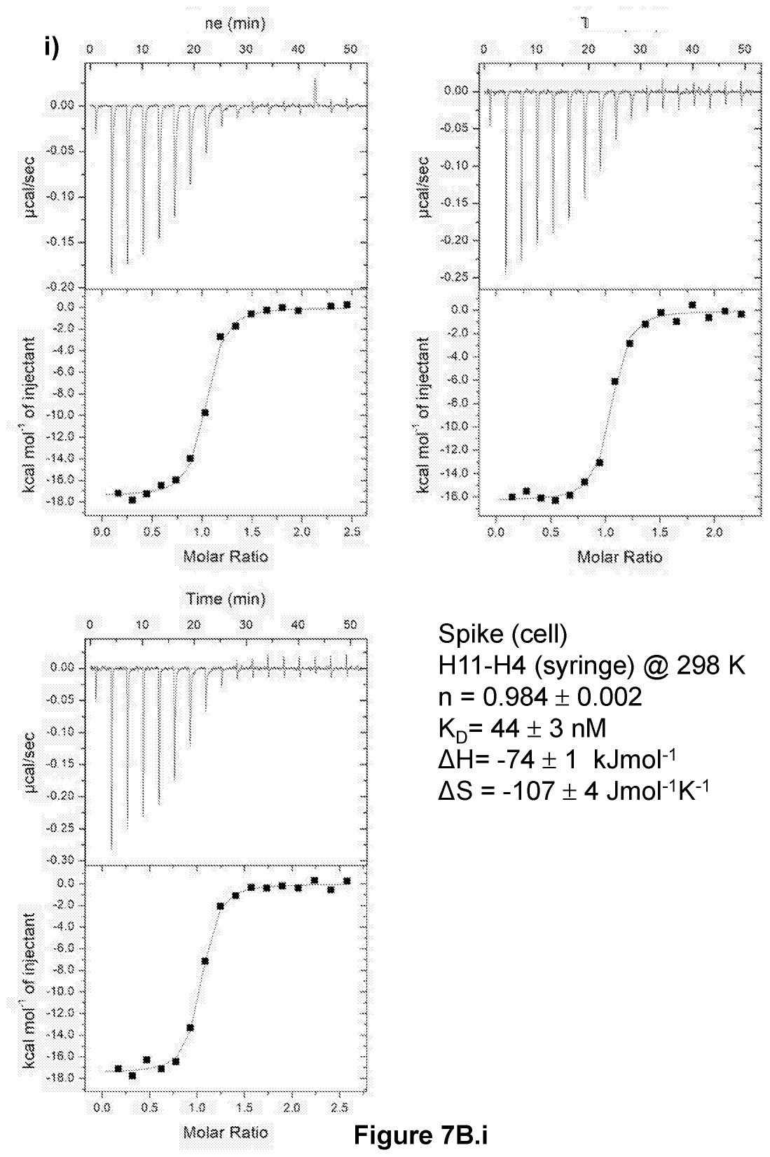

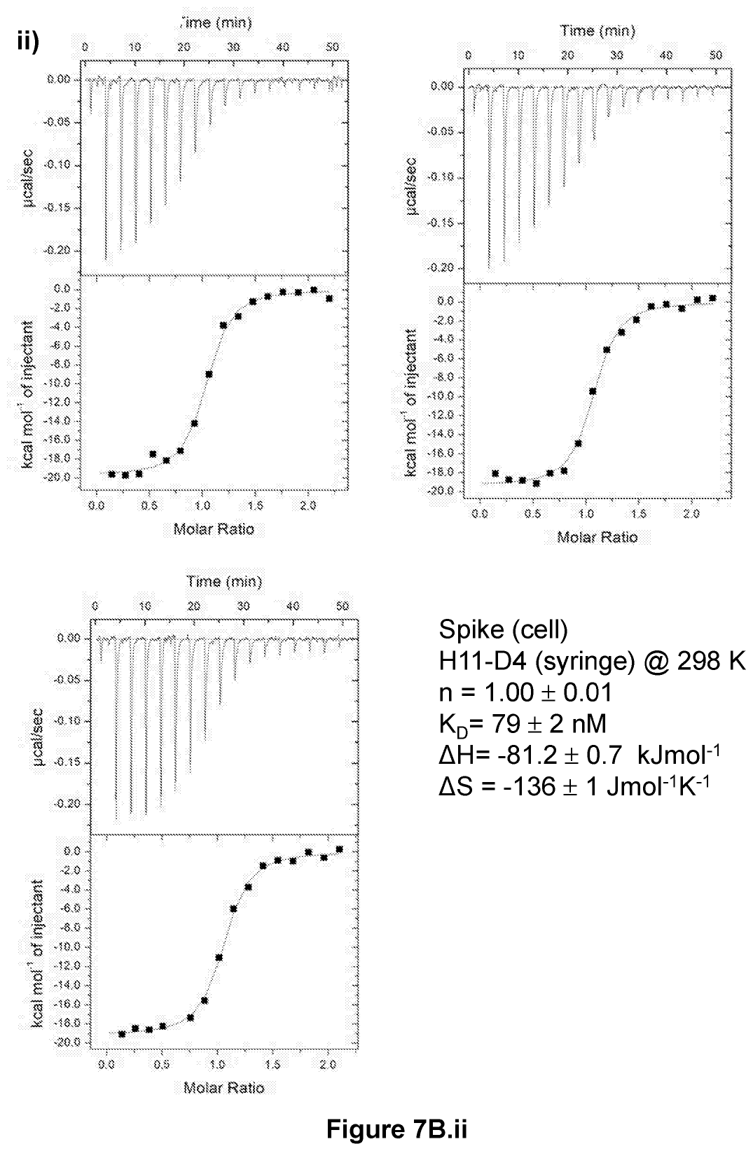

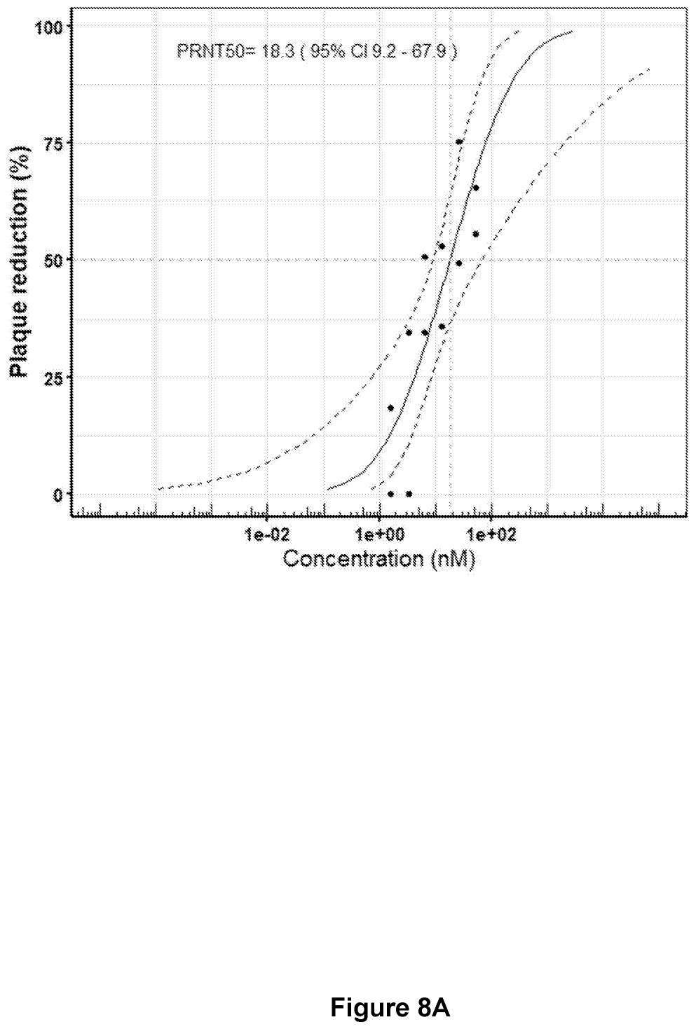

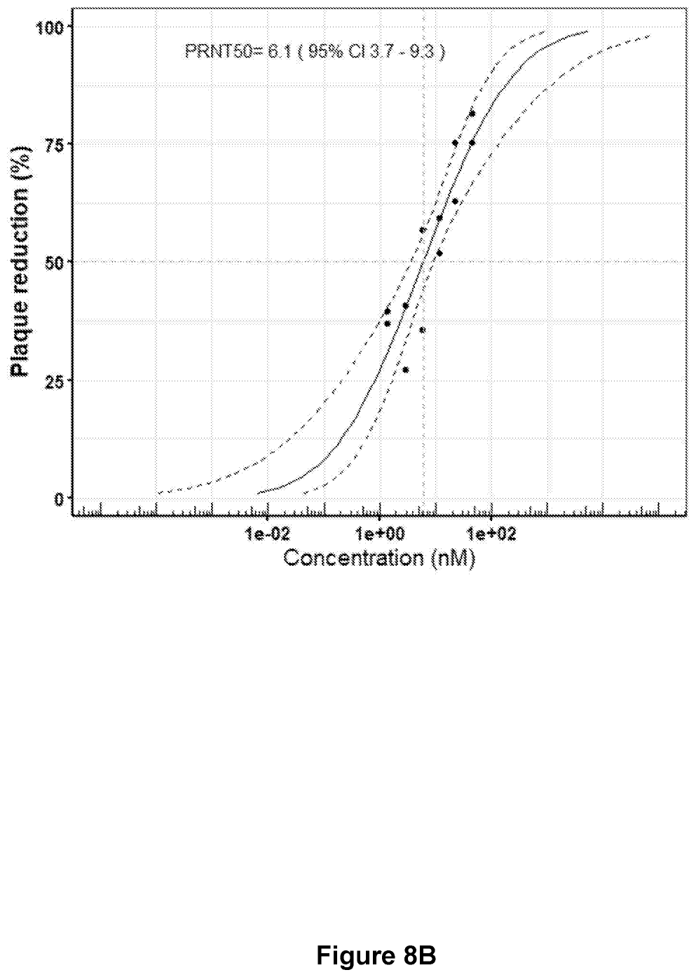

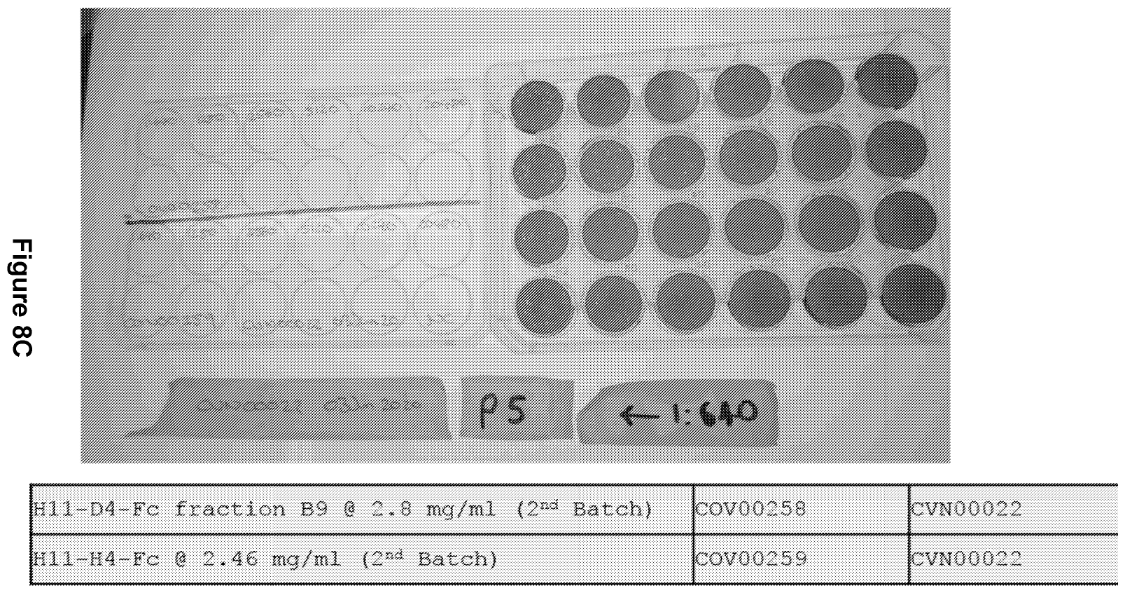

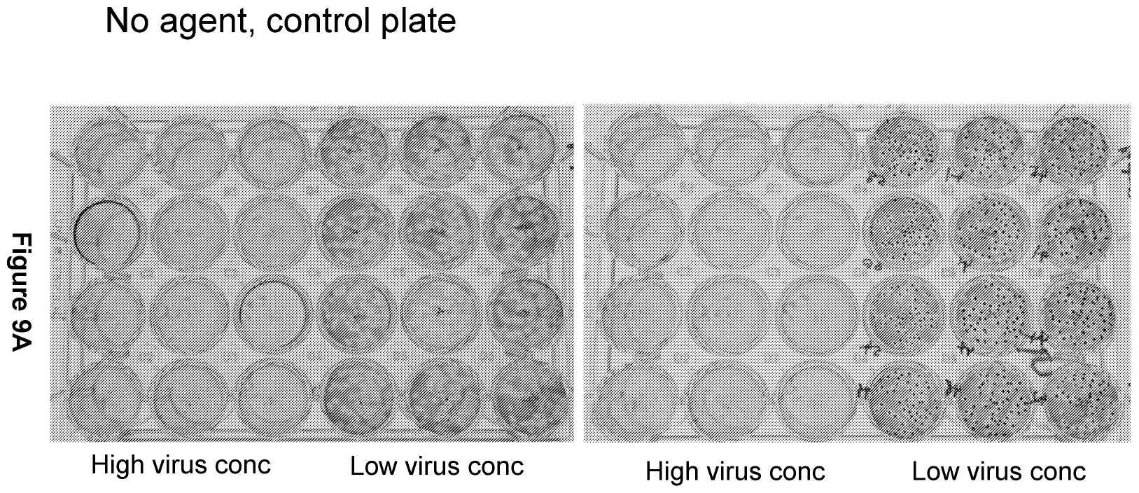

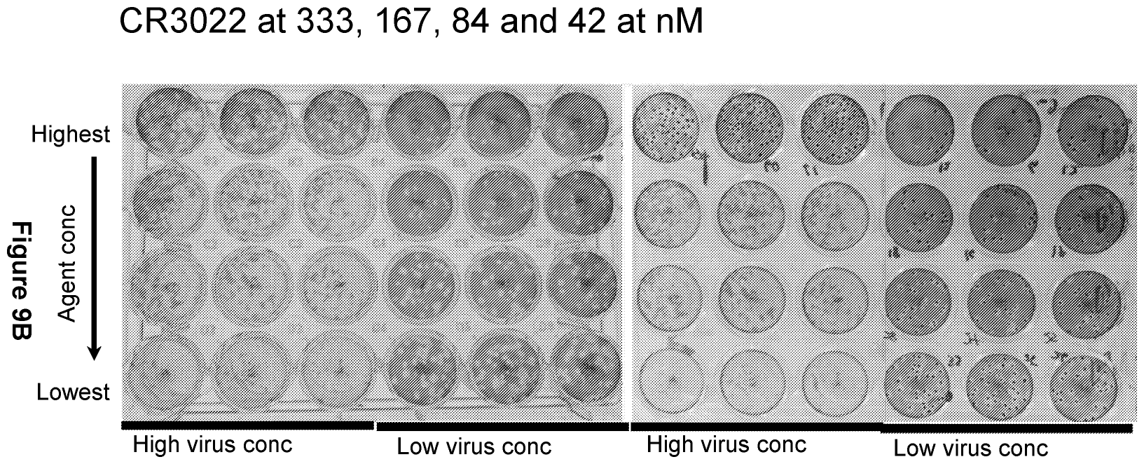

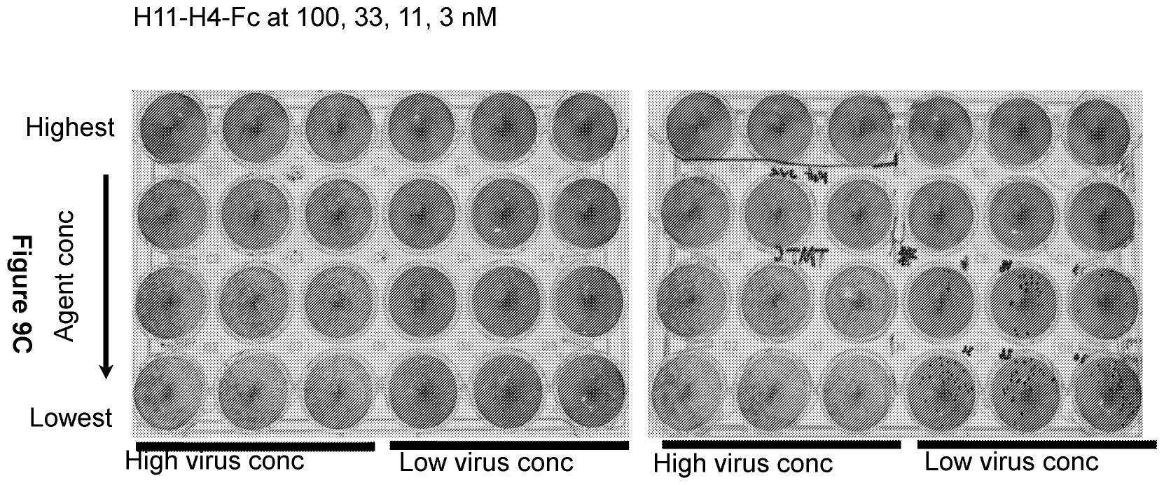

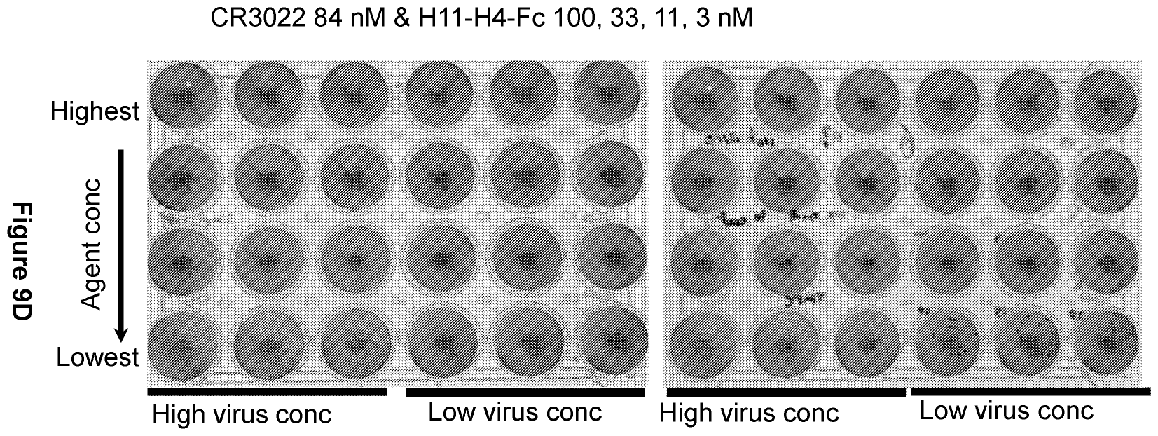

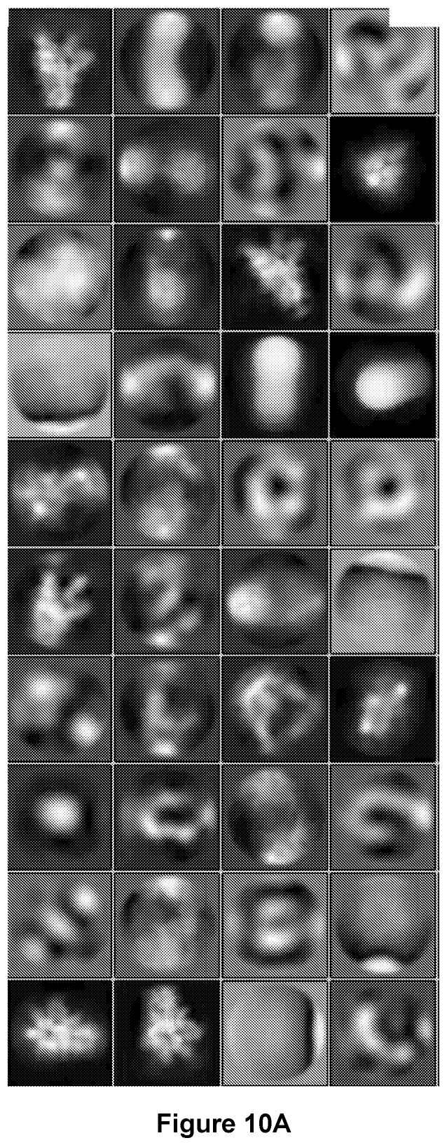

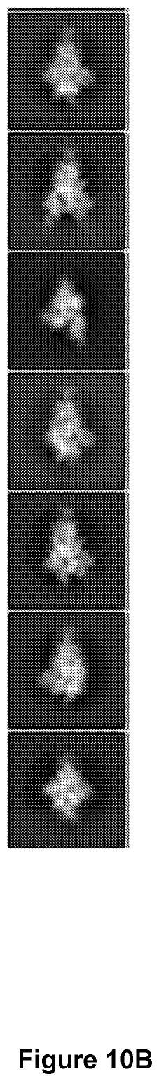

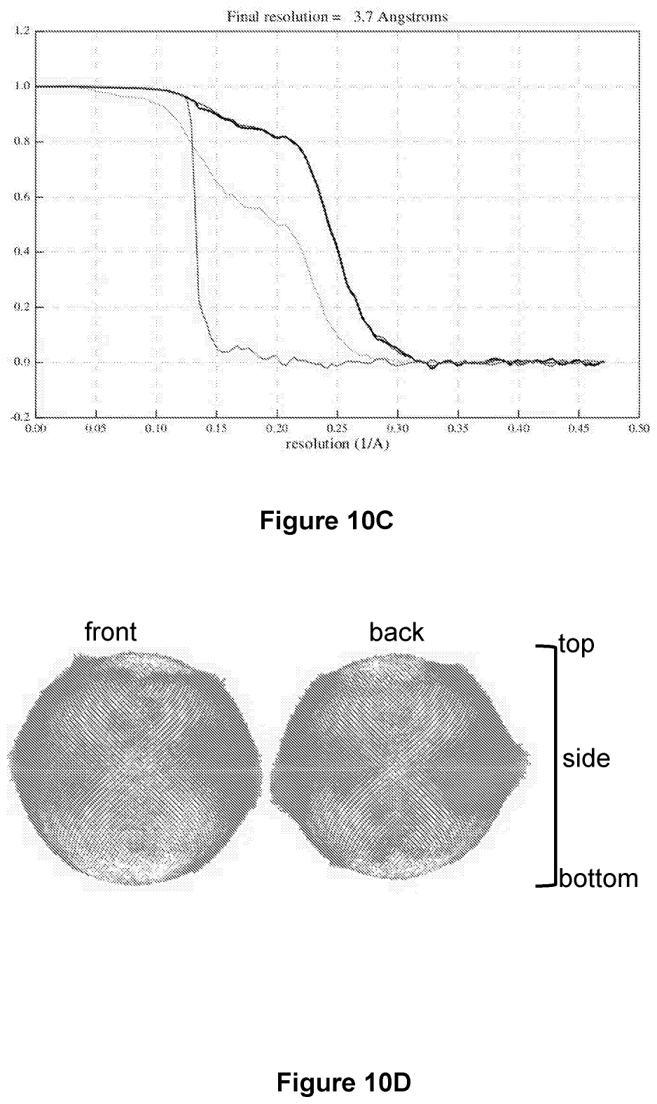

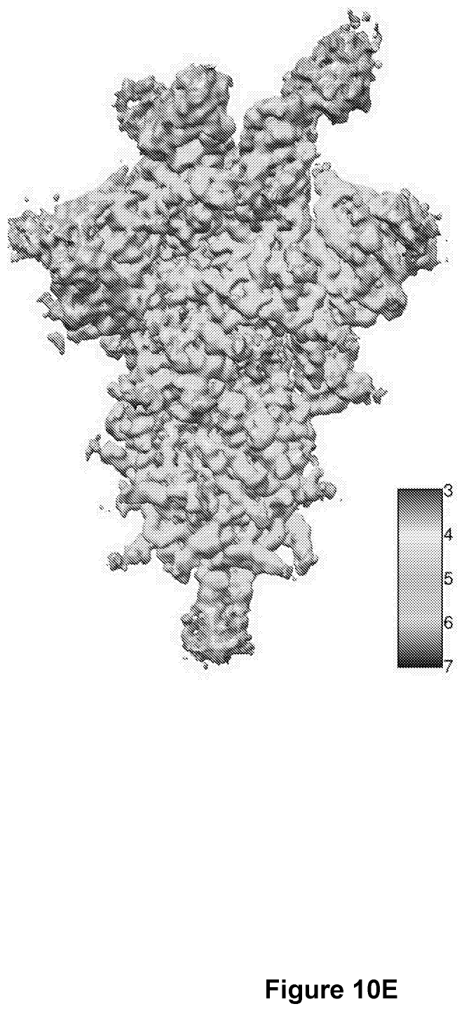

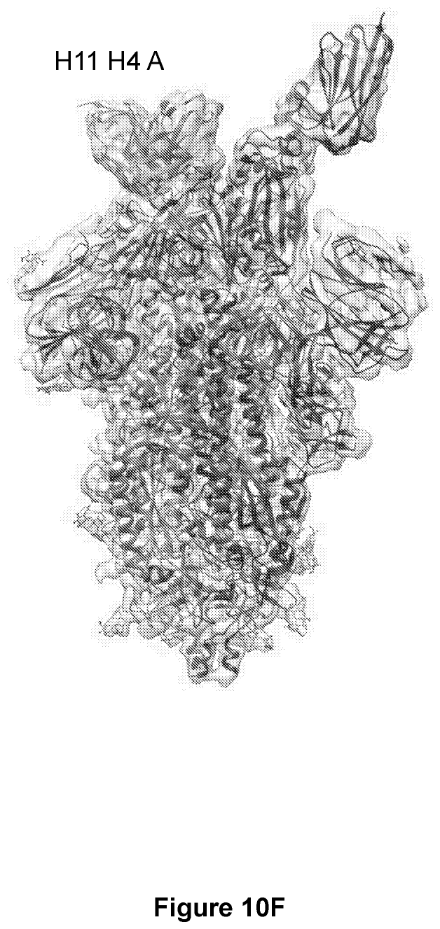

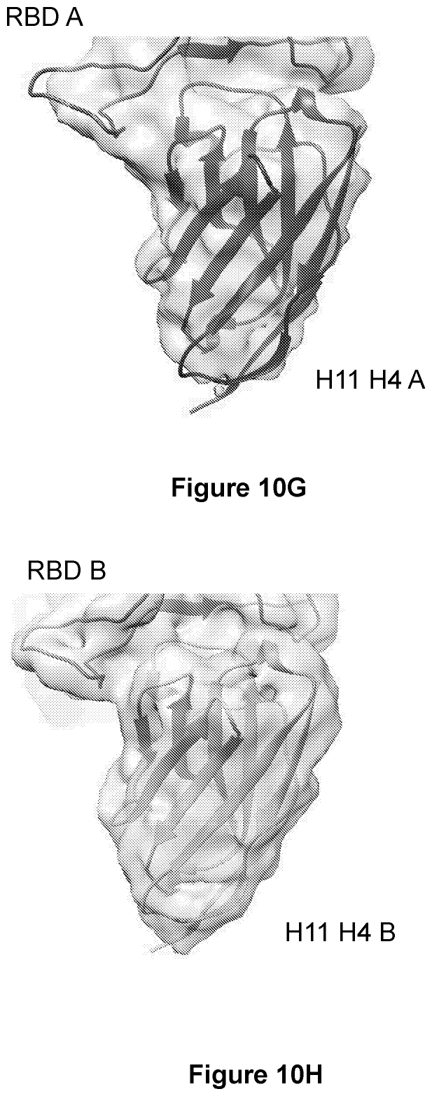

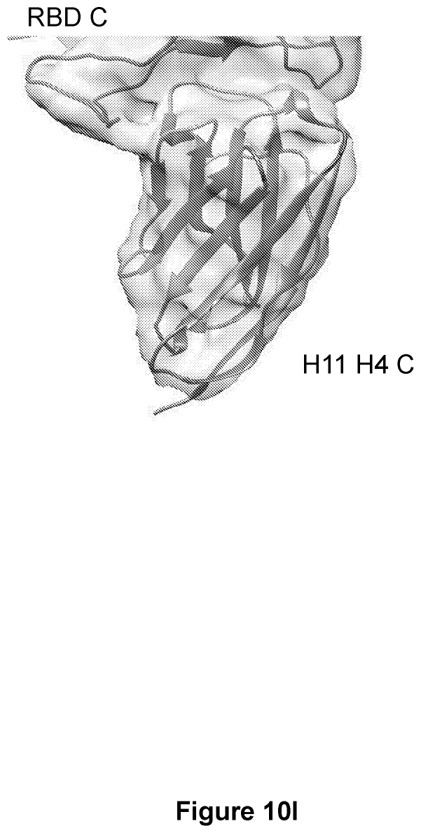

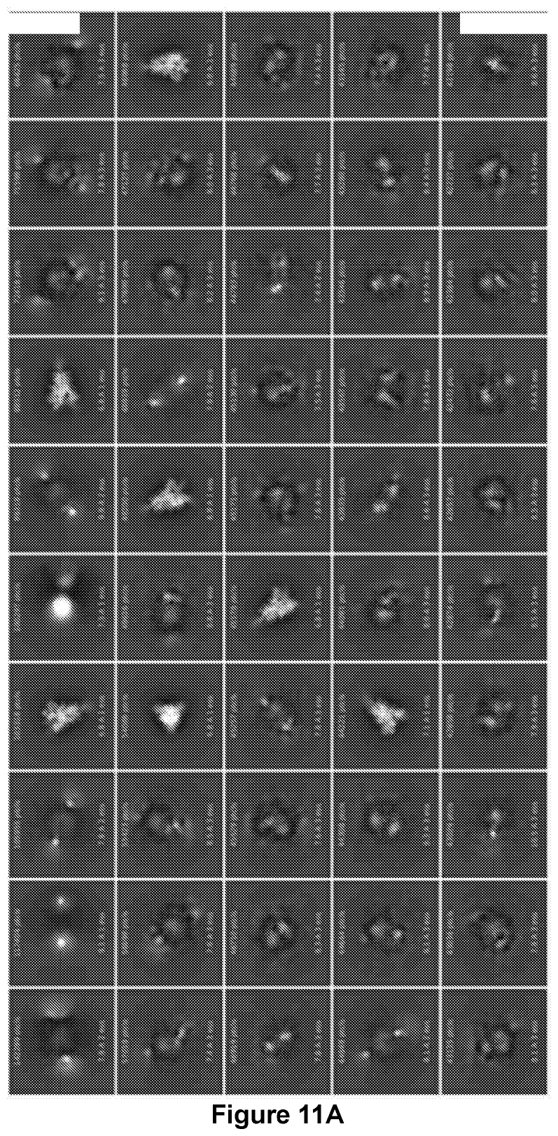

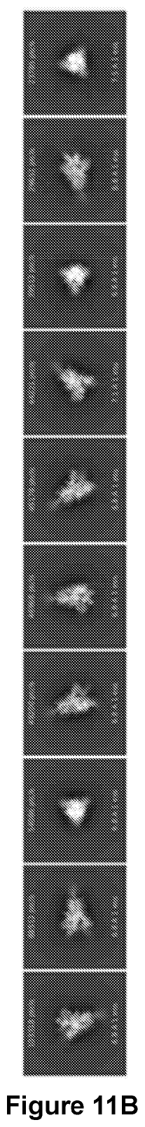

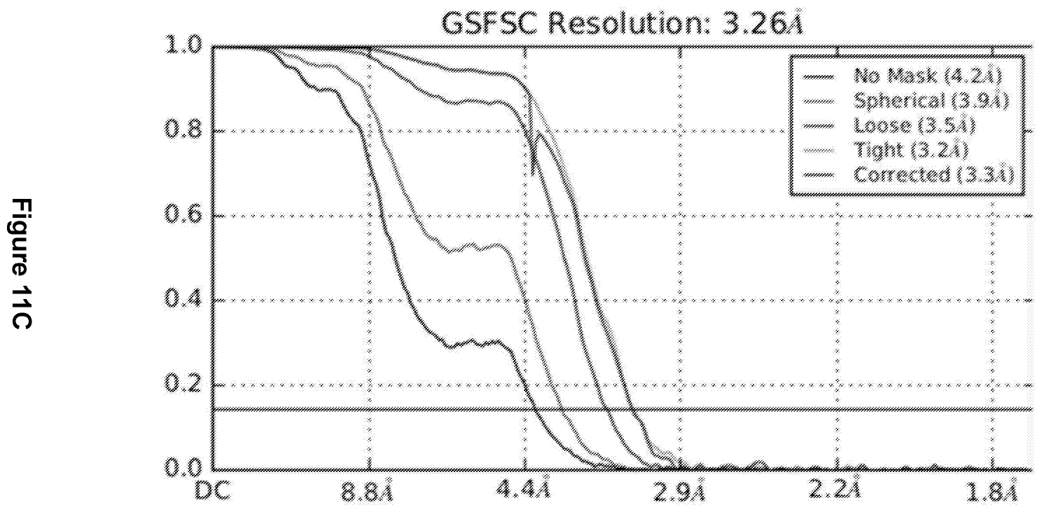

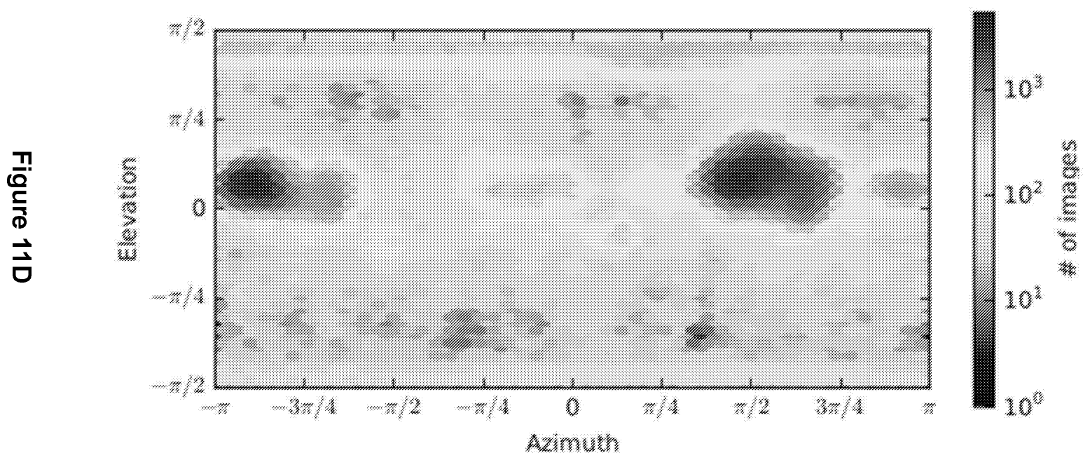

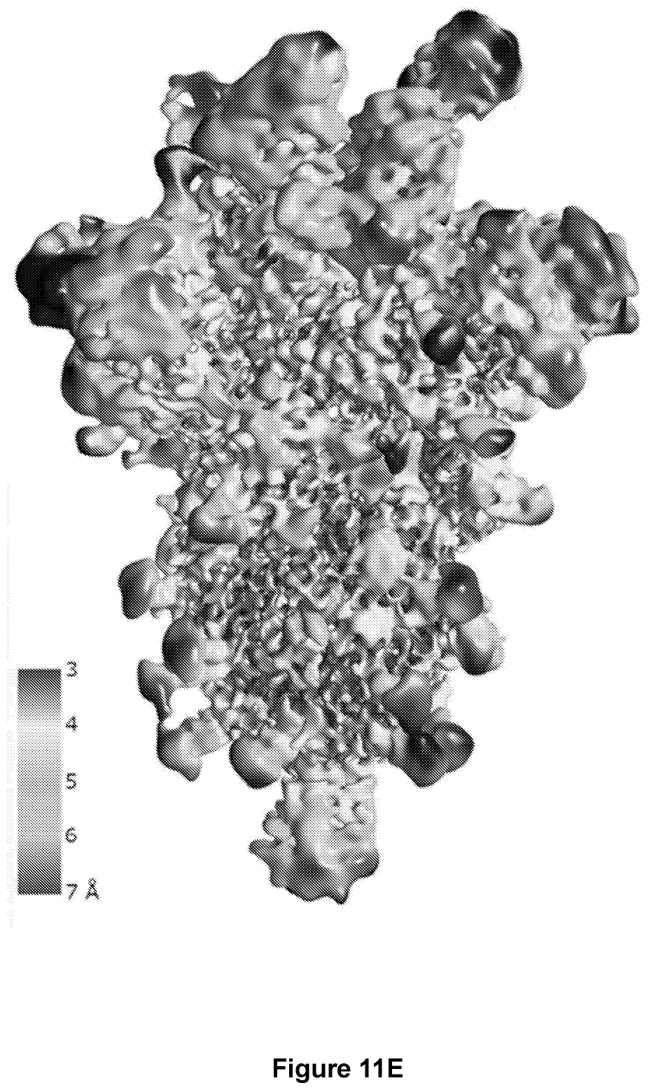

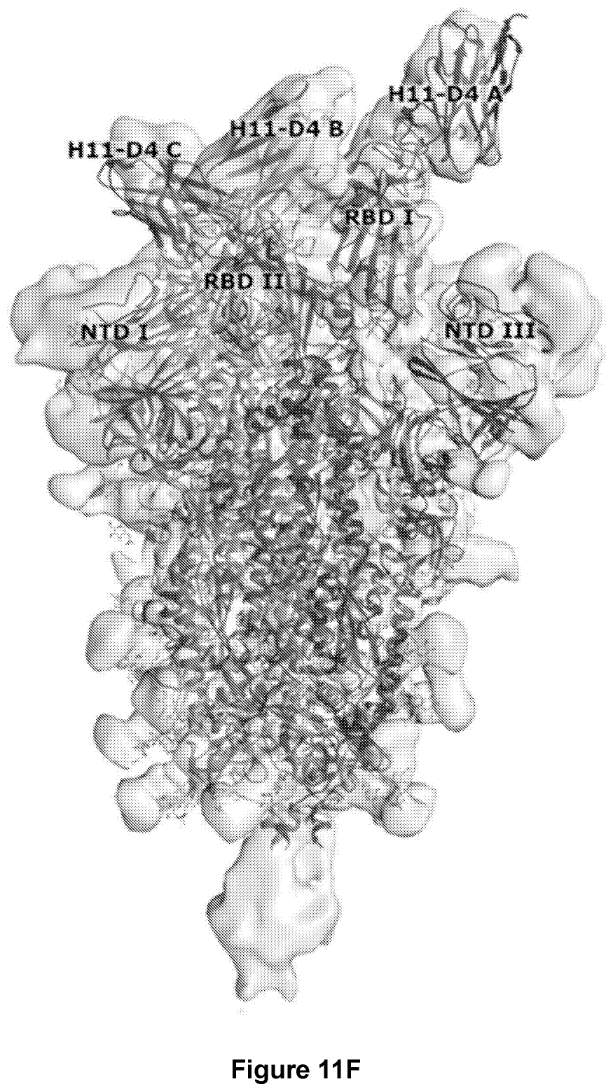

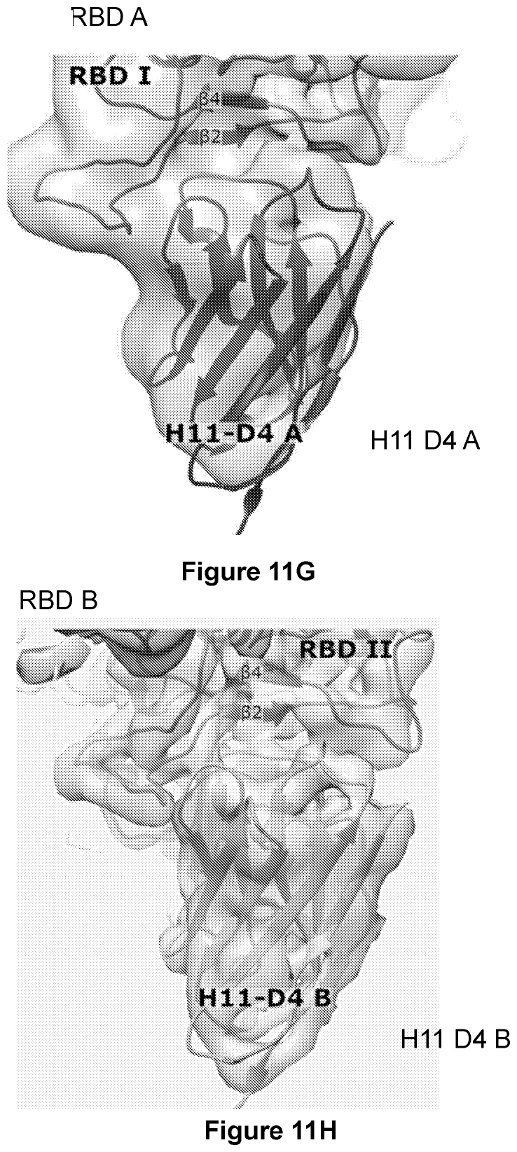

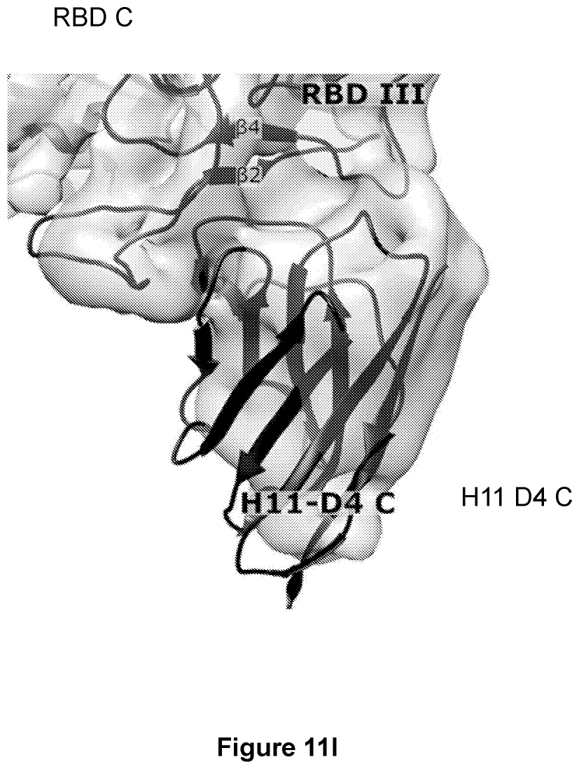

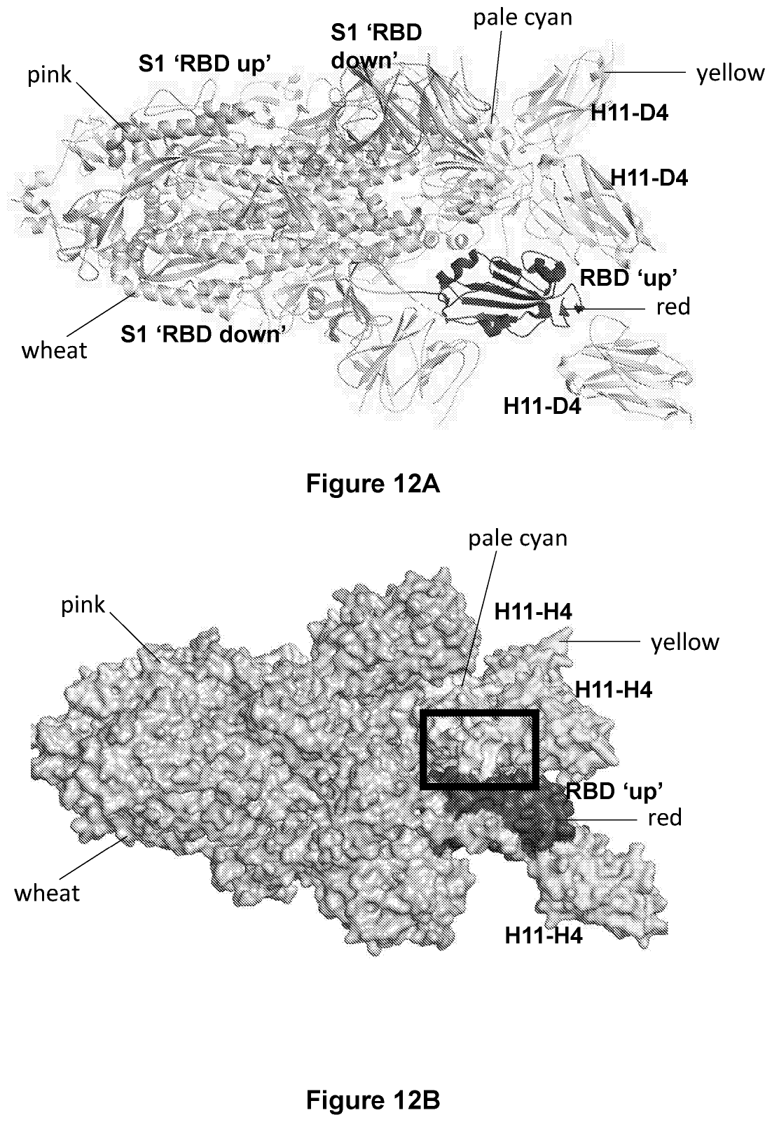

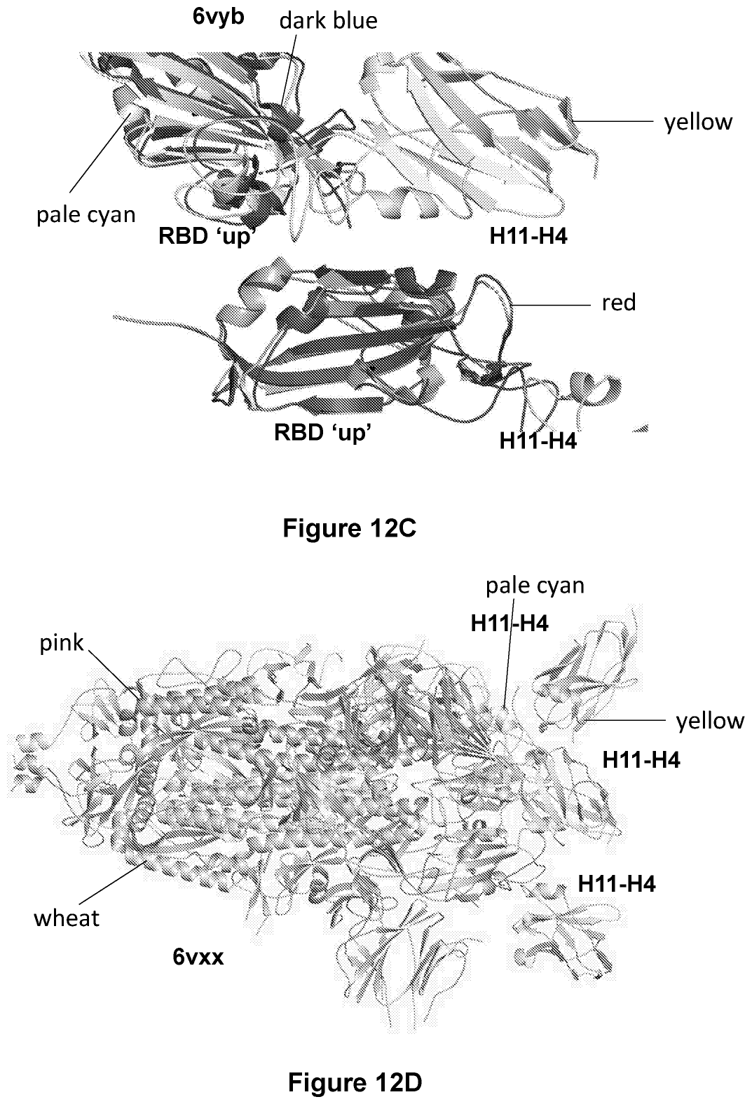

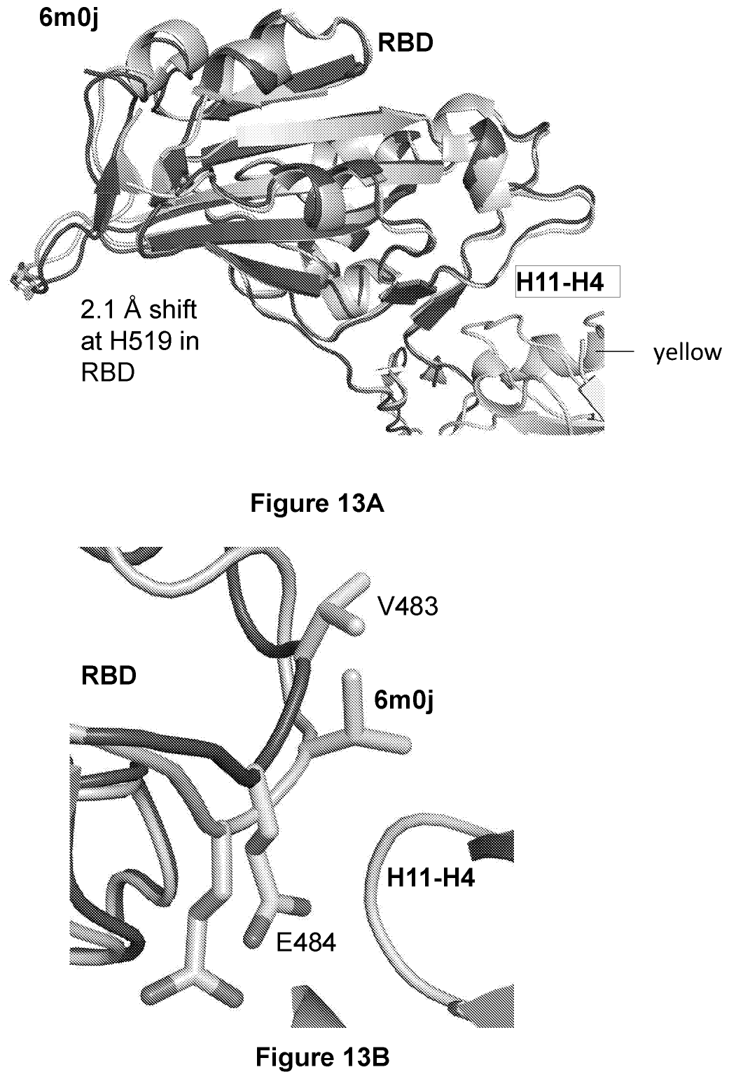

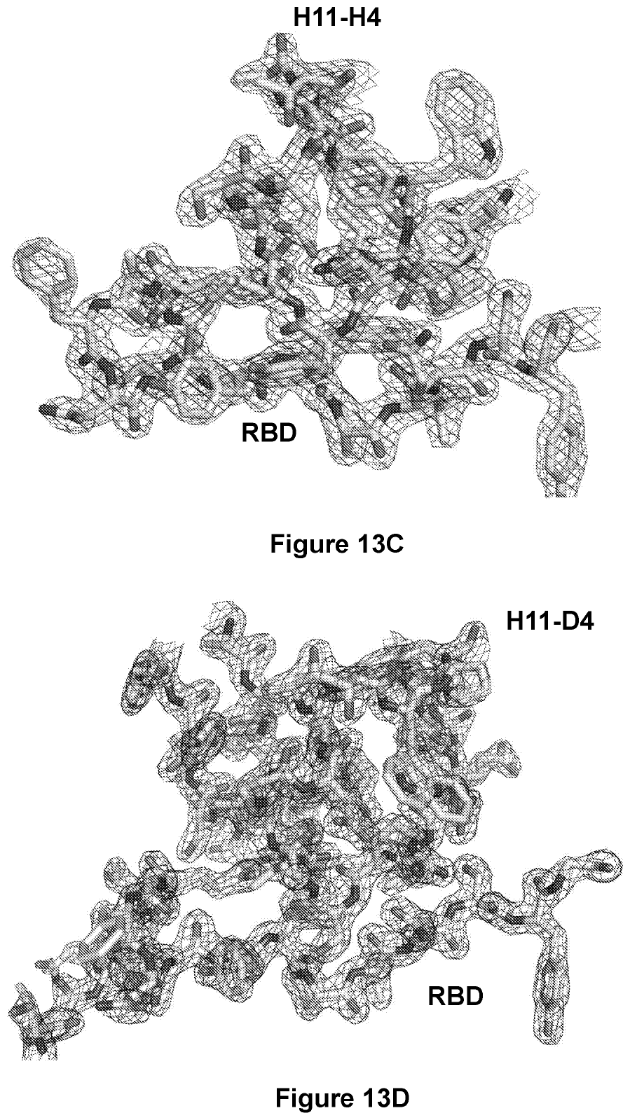

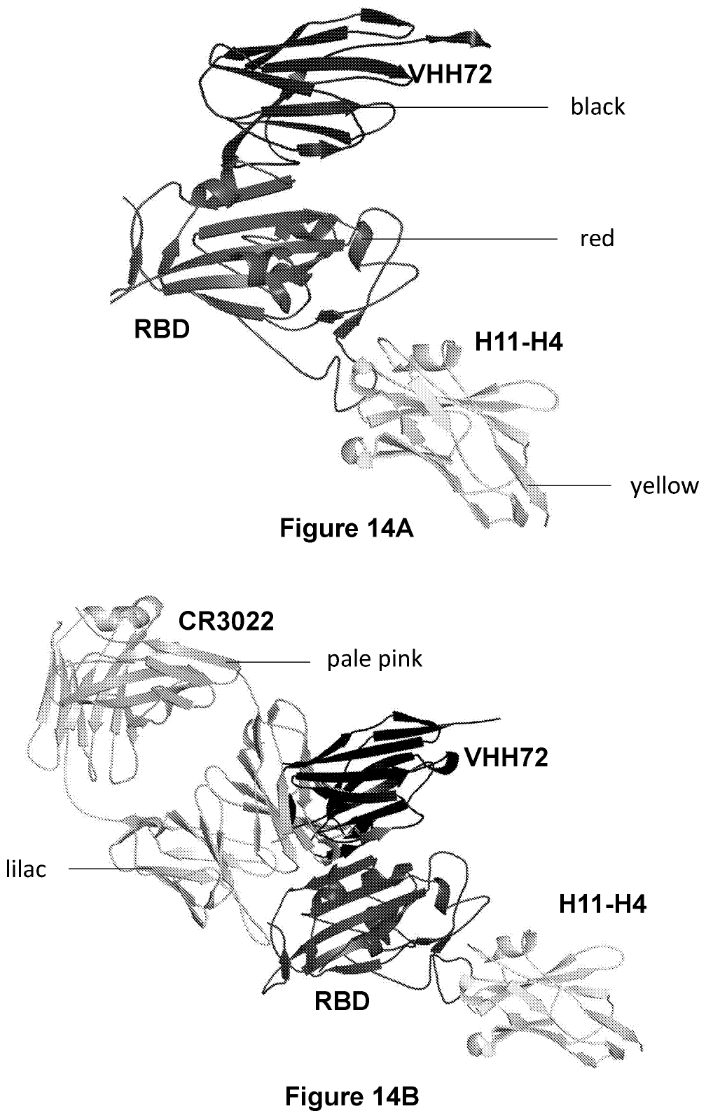

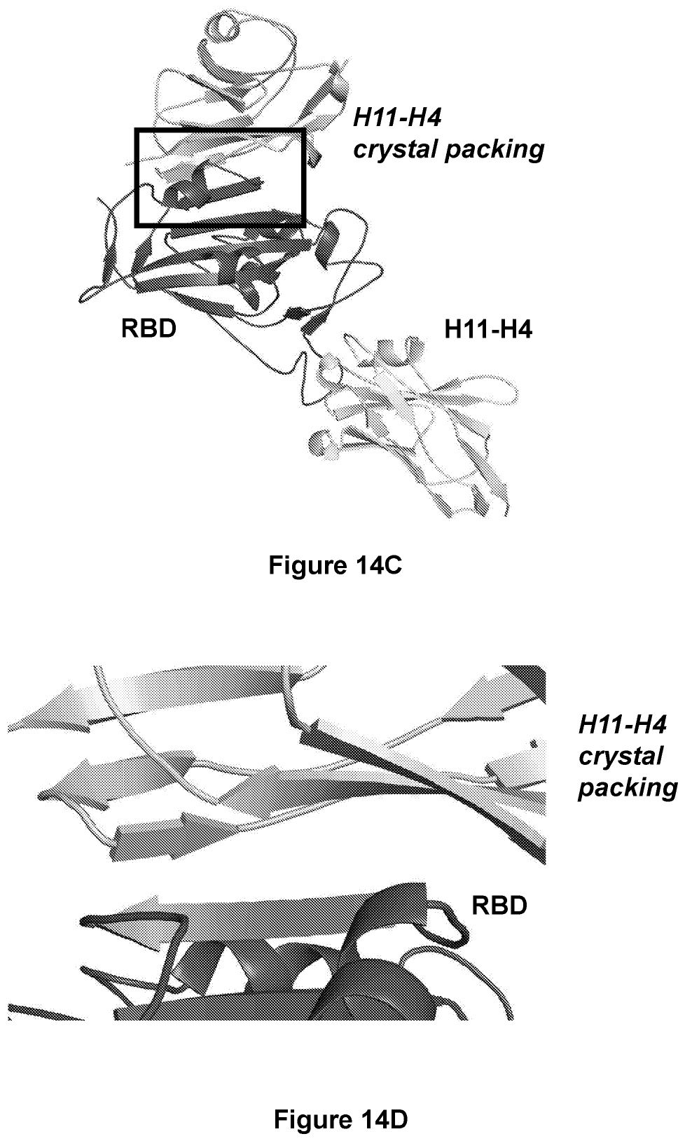

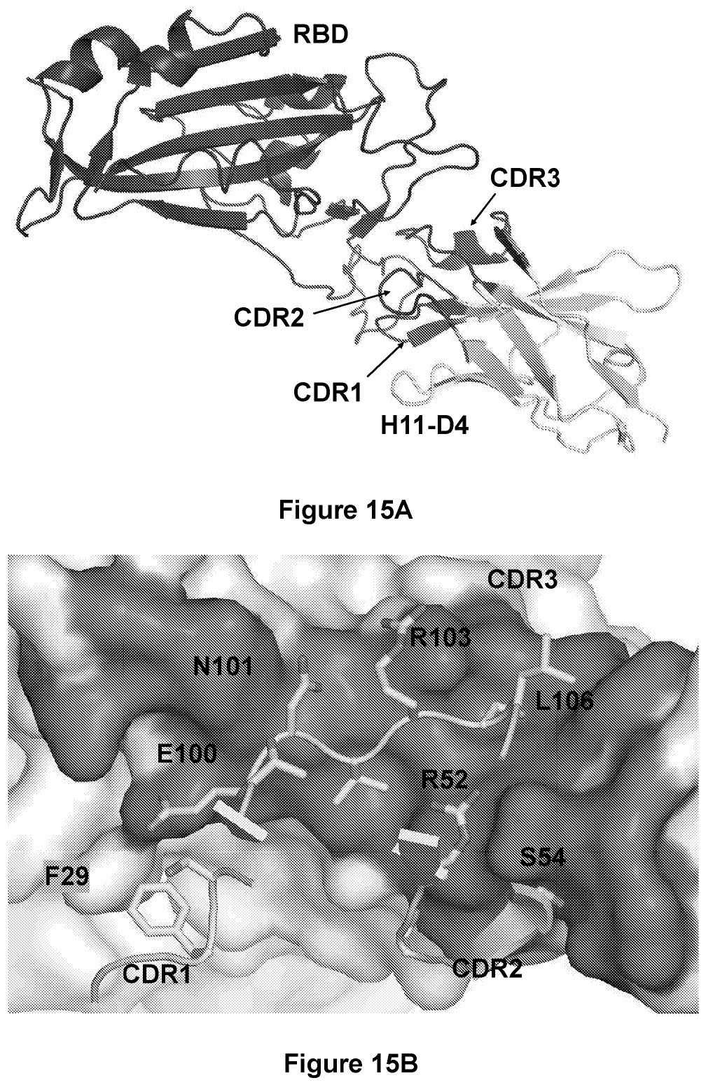

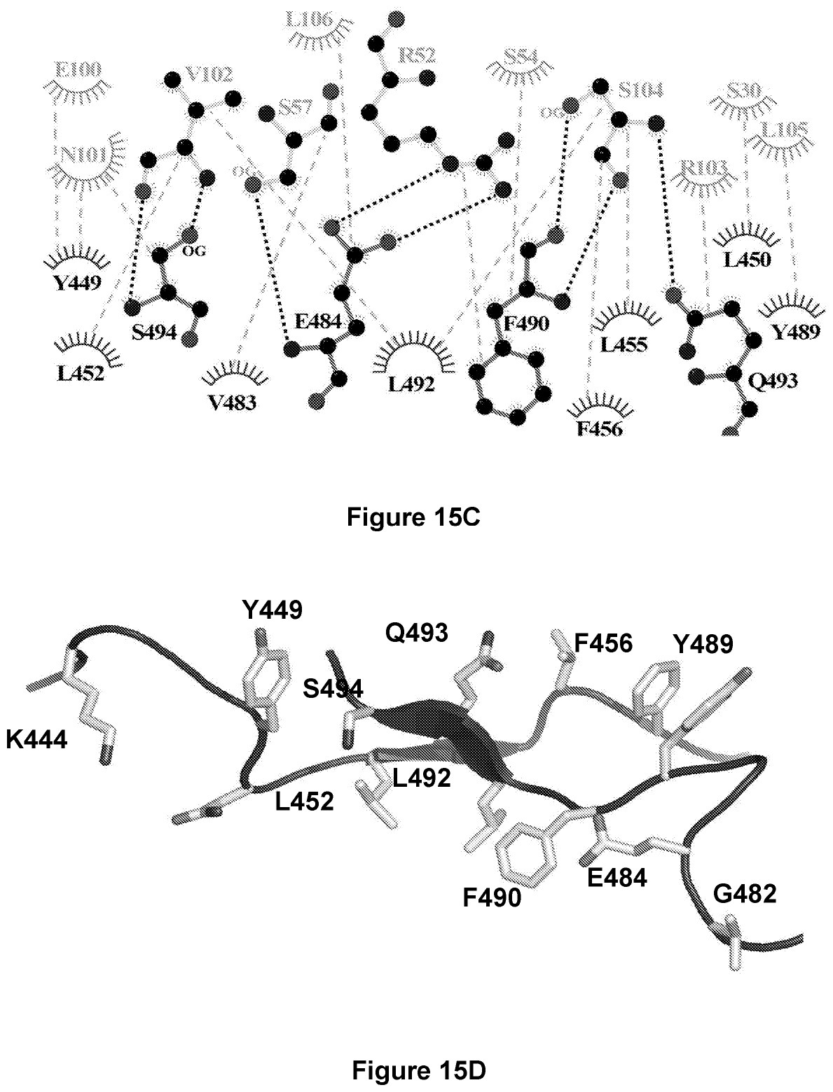

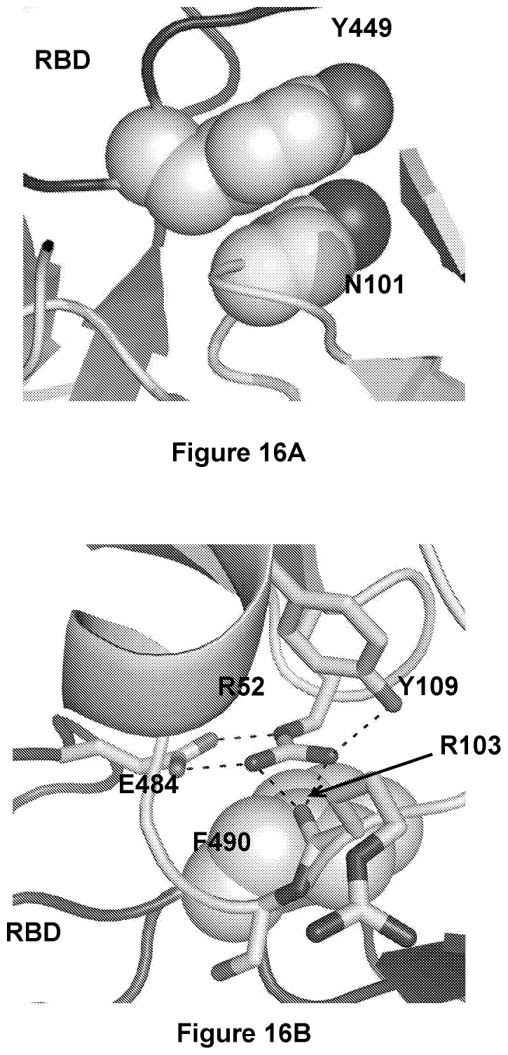

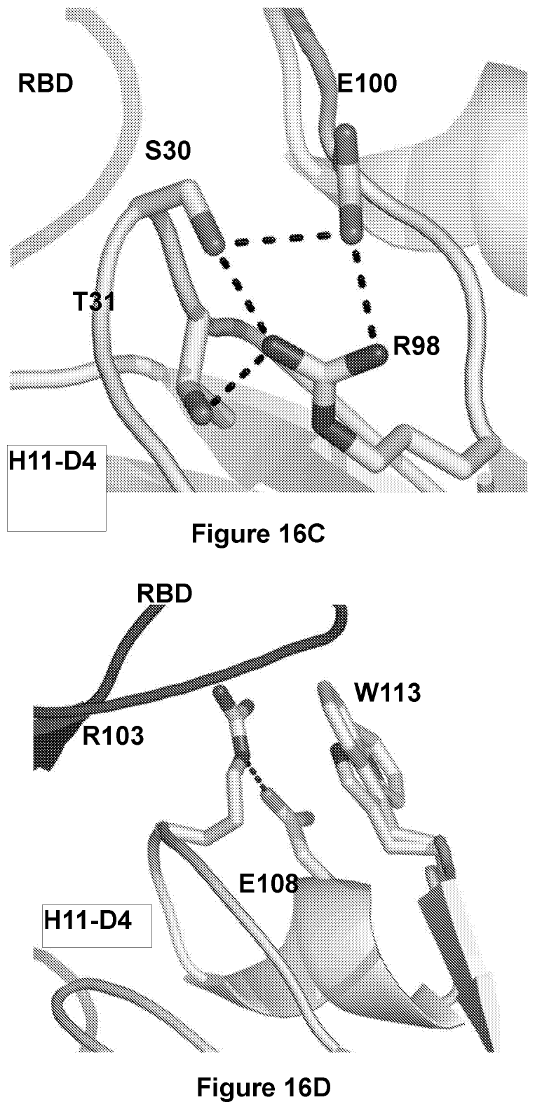

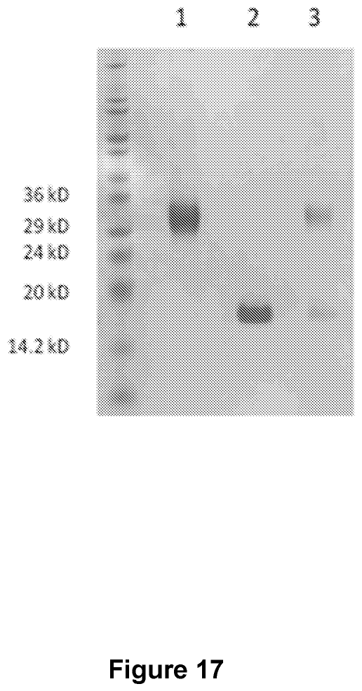

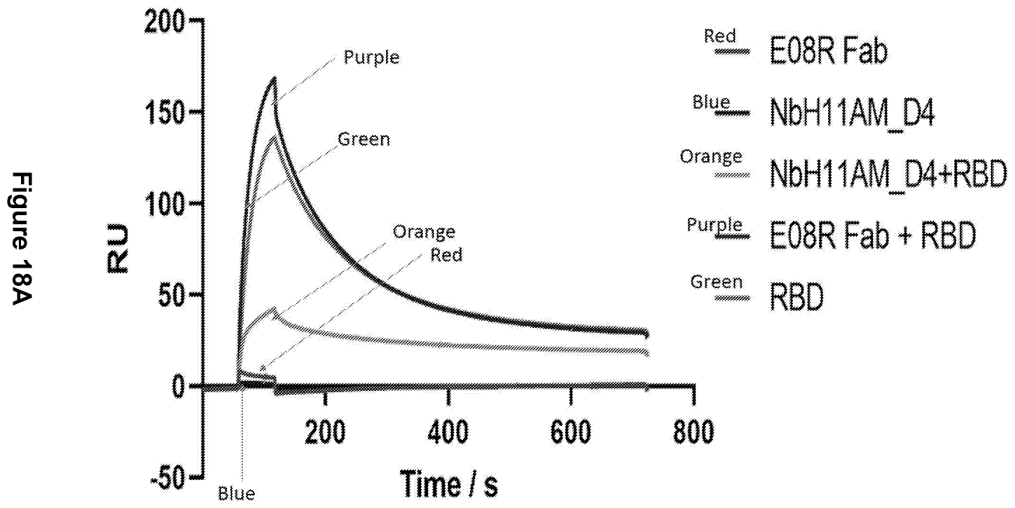

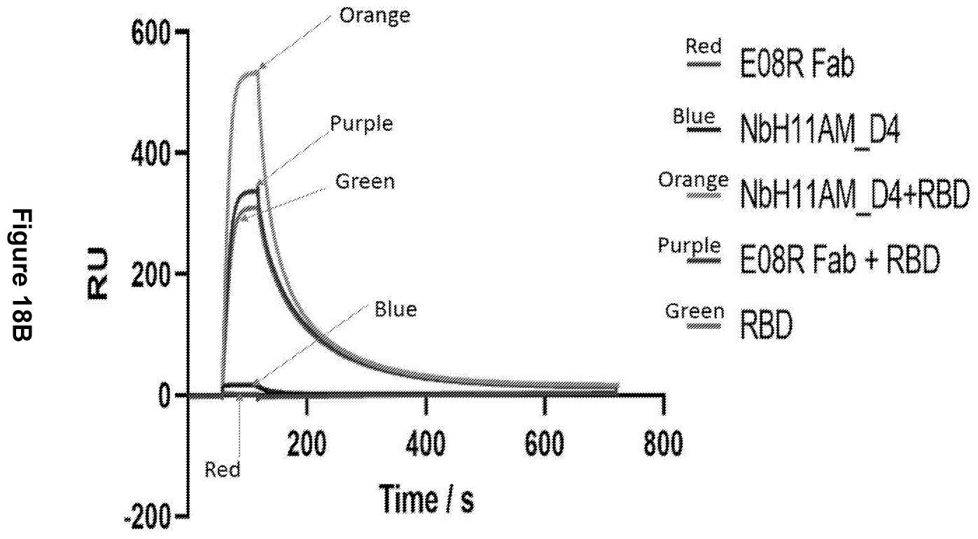

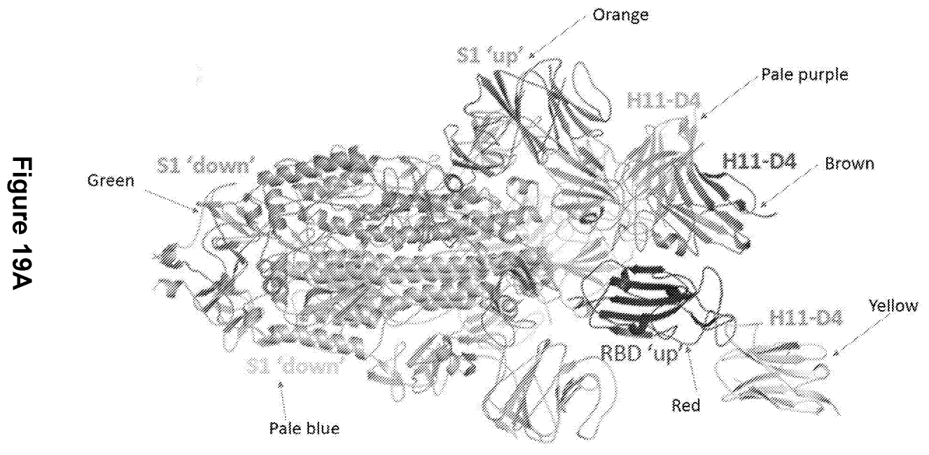

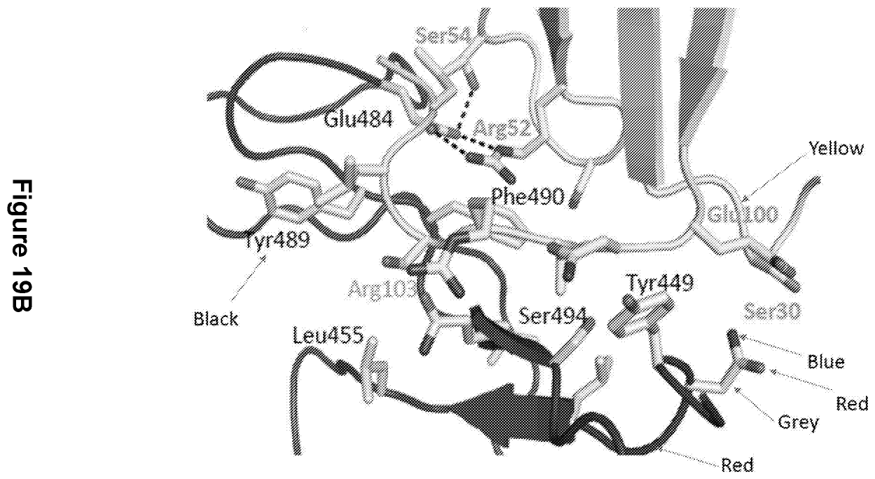

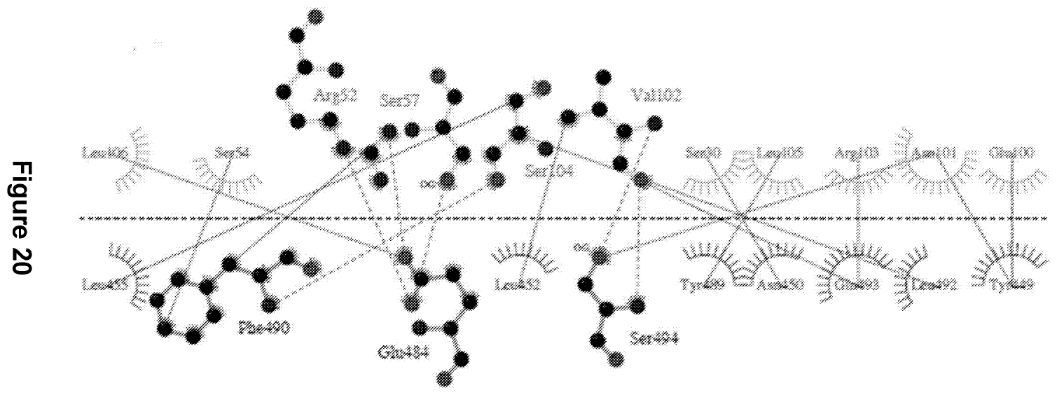

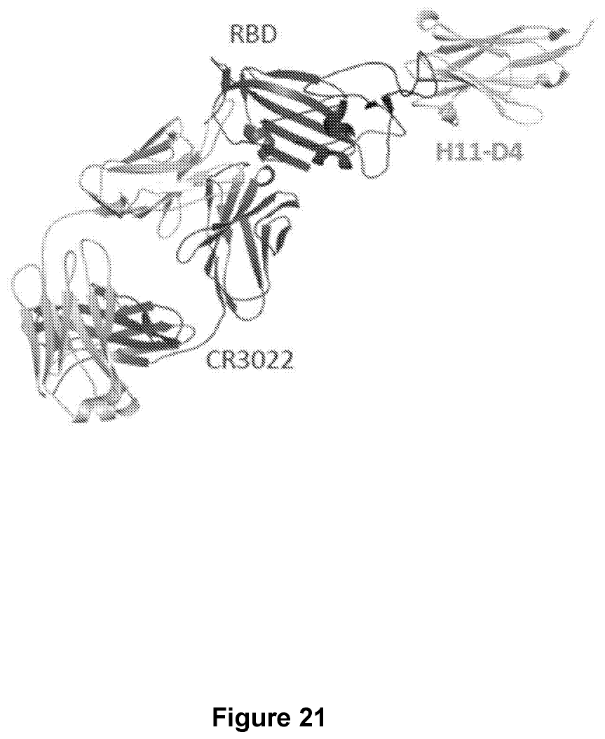

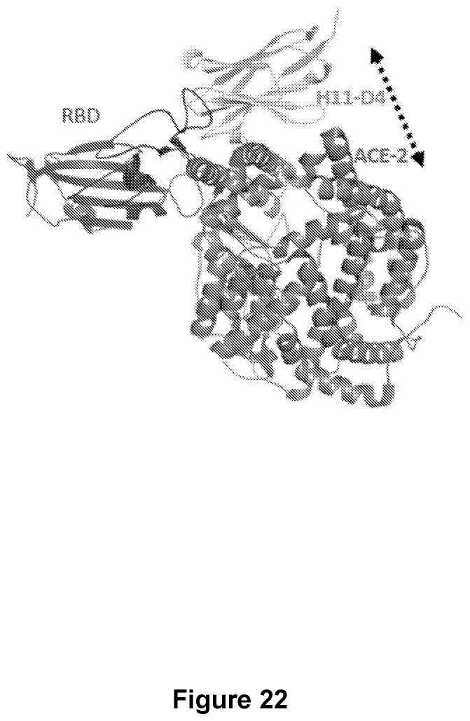

We used purified RBD of SARS-CoV-2 Spike to identify its binding partners in a naïve llama VHH library by in vitro phage display technology. We identified several nanobodies that bound to the RBD. The tightest binding nanobody, which we denoted H11, had a K_{D} of <1 μM (FIG. 6a, b). Using a random mutagenesis approach, we identified two affinity matured mutants, H11-D4 and H11-H4, which differ from H11 and each other at five residues within CDR3 (FIG. 1c, 2a). H11-H4 and H11-D4 were shown to bind RBD by surface plasmon resonance (SPR) with an estimated K_{D} of 5 nM and 10 nM respectively (FIG. 2b, FIG. 6c,d). We performed an SPR-based competition assay in which ACE2-Fc was immobilized and then binding of RBD was monitored in the presence or absence of H11-H4 or H11-D4; in a similar experiment, we also monitored Spike binding (instead of RBD). Both nanobodies inhibited the binding of both RBD and Spike to ACE2 (FIG. 2c, FIG. 6e). This suggested the nanobody epitope overlaps with the ACE2 binding site on RBD of Spike. When CR3022-Fc was immobilized and the binding of RBD measured, it was found that RBD binds to CR3022 whether H11-H4 or H11-D4 was present or not (FIG. 2d, FIG. 6f). This indicated that CR3022 and the nanobodies recognized non-overlapping epitopes on RBD. Repeating this experiment with Spike also showed binding in the presence and absence of H11-H4 (FIG. 2d, FIG. 6f).

The stoichiometry and thermodynamics of binding were characterized by isothermal titration calorimetry. H11-H4 binds to RBD with a K_{D} of 12±1.5 nM, H11-D4 with a K_{D} of 39±2 nM and both showed a 1:1 stoichiometry (FIG. 2e, FIG. 7a). When full length trimeric Spike was used, a single binding event was observed with a 1:1 nanobody:monomer (3:1 nanobody:Spike) stoichiometry and a K_{D} of 44±3 nM for H11-H4 and K_{D} of 79±2 nM for H11-D4 (FIG. 2f, FIG. 7b). Despite increased enthalpy, H11-D4 bound more weakly than H11-H4 as a result of an increased entropic penalty upon binding. The same enthalpy entropy compensation is observed when comparing Spike to RBD for both nanobodies. The Spike protein has been proposed to exist in multiple conformational states in solution²⁶, yet ITC showed a simple binding curve (FIG. 20. Either the nanobodies have bound equally well to all conformational states present or the equilibration between these states was faster than the binding event. The latter possibility seems less likely given the very high on rates (FIG. 2b).

### A Bivalent Fc—Nanobody Fusion Competes with ACE2 for RBD Binding

The nanobodies were fused to the Fc domain of human IgG1 to produce a homodimeric chimeric protein capable of bivalent binding (FIG. 3a). The ability of these constructs to block ACE2 binding to RBD was tested in two assays.

In the first assay, MDCK-SIAT1 cells stably expressing human ACE2 (MDCK-ACE2) were seeded on plates and the ability of various analytes (H11-H4-Fc, H11-D4-Fc, ACE2-Fc, CR3022¹⁸ and VHH72-Fc) to block binding of RBD was measured (FIG. 3b). VHH72²⁵ is a nanobody isolated from a llama immunized with Spike from SARS-CoV-1 which is cross reactive against Spike from SARS-CoV-2. The MDCK- ACE2 cell binding assay yielded an IC₅₀ of 61 nM for H11-H4-Fc, 161 nM for H11-D4-Fc and 262 nM for VHH72-Fc²⁵.

In the second competition assay, analytes (H11-H4-Fc, H11-D4-Fc, ACE2-Fc, CR3022¹⁸, VHH72-Fc²⁵) were assessed for their ability to block ACE2 binding to MDCK cells that expressed RBD on their surface (FIG. 3c). This assay yielded an IC₅₀ of 34 nM for H11-H4-Fc, 28 nM H11-D4-Fc and 33 nM for VHH72-Fc²⁵. As expected, CR3022 does not show a strong response in either assay since it does not block the RBD-ACE2 interaction^{17,18}.

### H11-H4-Fc and H11-D4-Fc Neutralize Virus

The chimeric fusions were tested in a plaque reduction neutralization test at the Public Health England Laboratory for SARS-CoV-2 virus, and showed an ND₅₀ of 6 nM for H11-H4-Fc (95% confidence interval (CI) 3-9 nM) and ND₅₀ of 18 nM for H11-D4-Fc (95% CI 9-68 nM) (FIG. 3d, FIG. 8). H11-H4-Fc neutralization was replicated at Oxford University and yielded an ND₅₀ of 4 nM. CR3022 was used as a positive control, and under these conditions an ND₅₀ of 93 nM was observed, similar to a previous report¹⁸ (FIG. 3e). The raw plates are shown in FIG. 9 and we observed a small plaque phenotype in the presence of H11-H4-Fc but not in the positive control CR3022. This is particularly surprising as it shows that the ability of H11-H4-Fc to prevent non-neutralized virus from spreading.

It should be noted that our assay method did not remove virus and neutralizing agent after incubation with cells, in line with UK standards. Some labs have reported a neutralization assay protocol where virus and neutralizing agent are removed during the assay, precise protocol differences may be responsible for the reported difference in CR3022 neutralization^{17,18}.

### Structures of Nanobody—Spike and Nanobody—RBD Complexes

The nanobodies were each incubated at room temperature with a purified prefusion-stabilized ectodomain of the SARS-CoV-2 Spike protein¹³ (Spike(trimer):nanobody=1:4) and then vitrified on cryo-EM grids. The cryo-EM single particle structure of this variant of Spike has been shown to be trimeric with a predominantly ‘up—down—down’ arrangement of the three RBDs¹³. After data collection and processing (Table 4, FIG. 10, 11) the maps clearly identified additional density at all three RBDs in the H11-D4 and H11-H4 complexes (FIG. 10, 11). Improvement in the Coulomb potential maps allowed fitting of the nanobody into the additional density at each RBD in both structures (Table 4, online Methods, FIG. 4a and FIG. 10, 11, 12a). The density for the nanobody bound to the ‘up’ RBD is weak, but still clearly discernible, whilst the density for the nanobodies bound to the ‘down’ RBDs is clearer (FIG. 10, 6). The structures of H11-H4-Spike (FIGS. 4a) and H11-D4-Spike complexes (FIG. 12a) are indistinguishable given their resolution (Table 4). We focus our description here on the complex with the higher affinity nanobody, H11-H4.

The region of the RBD in contact with the nanobody is ordered in the nanobody complex but is disordered in the EM pre-fusion stabilized holo Spike structures (PDB 6VSB, 6VYB, 6VXX)^{13,26}, precluding detailed analysis. However, we noted that in the nanobody—Spike complex, the ‘up’ RBD (subunit A) makes contacts with the nanobody that is bound to ‘down’ RBD (subunit C) (FIG. 12b); contacts that are absent in the holo Spike. These contacts have resulted in shifts of the RBD domains when compared to the non-complexed form^{13,26} (FIG. 12c). Matching previous reports²⁶, we have seen a mixture of two forms (‘three down’ and ‘one up two down’) on the grids for holo Spike protein. In the presence of the nanobody, only the ‘one up two down’ form was observed, indicating that nanobody binding reduced conformational heterogeneity. We suggest the additional interactions are responsible for this observation and for the higher enthalpy and greater entropic penalty observed for nanobody binding to Spike when compared to RBD (FIG. 2e, f).

Nanobodies rely on three variable loops denoted CDR1, CDR2 and CDR3 to form the antigen-binding site (FIG. 1c). To gain insight into the molecular basis of recognition, crystal structures of the H11-H4-SARS-CoV-2 RBD complex and the H11-D4-SARS-CoV-2 RBD complex were determined to 1.85 and 1.80Å resolution respectively (Table 5). Both crystal structures have a single copy of the complex in the asymmetric unit. Superposition of the two complexes has confirmed that both nanobodies recognize the same epitope (FIG. 4b). Comparison of the structures shows that the entire complex superimposes with an r.m.s.d. 1.0Å over 322 Cα atoms, but the individual RBD's superimpose with an r.m.s.d. 0.5Å over 195 Cα atoms and the individual nanobodies with an r.m.s.d. 0.4Å over 127 Cα atoms. The higher r.m.s.d. for the complex has arisen from a 7° pivot motion of the nanobodies with respect to each other (FIG. 4c). Given the very high degree of similarity between the complexes, we again focused the description on the H11-H4-RBD complex (FIG. 4d). There are differences compared to the H11-D4-RBD complex, due to sequence changes in the CDR3 loops, and a detailed description of the H11-D4-RBD interface is provided in FIGS. 15-16 and ‘Structural analysis of H11-D4 RBP complex’.

In the complex, CDR1 loop of H11-H4 has contributed very little to the interface (FIG. 4e). From CDR2, residues Arg52, Ser54 and Ser57 have made contacts with RBD (FIG. 4d,e). From CDR3, His100 to Leu106, the region modified during maturation, made contacts with RBD (FIG. 4d,e). The surface on RBD which contacts H11-H4 is formed by Lys444 to Phe456 and Gly482 to Ser494 (FIG. 4f). These two stretches of RBD sequence comprise 90% of buried surface area and make all the hydrogen bonds with H11-H4 (FIG. 4g). In addition to these direct contacts, there are multiple bridging water molecules. To our surprise the PISA server²⁷ does not identify either nanobody—RBD complex as stable.

Arg52 from CDR2 of H11-H4 was found at the heart of a network of interactions, including RBD residues Glu484 with which it made a bivalent salt link and Phe490 with which it made a π-cation interaction²⁸ (FIG. 4h). Arg52 also forms hydrogen bonds to the backbone carbonyl of Ser103 and side chain of Tyr109 (FIG. 4h) that may stabilize the conformation of the CDR3 loop. The seven-residue stretch of H11-H4 CDR3 region, which varied during maturation, contributes over 60% of the surface area buried by the complex and makes five hydrogen bonds to RBD (FIG. 4g).

Using the H11-D4-RBD complex, we created a model of three nanobodies bound to ‘down’ (closed) form of the Spike²⁶ (FIG. 12d). This model does not disclose any clashes, suggesting that the nanobody would bind to the Spike protein in all its conformational states consistent with the simple ITC curve (FIG. 2e).

### The Nanobody Epitope Compared to Other RBD Binders

Superposition of the RBD—ACE2 complex^{29,30} upon the H11-H4-RBD complex reveals that H11-H4 would, consistent with biophysics (FIG. 2c), plate assays (FIG. 3b, c) and neutralization experiments (FIG. 3d), prevent ACE2 binding to RBD (FIG. 5a). This is due to van der Waals clashes, principally between regions of H11-H4 that are not in contact with the RBD and regions of ACE2 (also not in contact with RBD) (FIG. 5a). Interestingly the contact surface of H11-H4 on RBD shows only a small overlap with the ACE2 contact surface (FIG. 1b, FIG. 5b). Comparison with the RBD—ACE2 complex29,30 reveals that residues 445-500 of RBD appear to move as a rigid unit upon H11-H4 binding (FIG. 13a). The structure of the loop centered at Val483 of the RBD has changed upon the binding of H11-H4 (FIG. 13b).

Given the potential for additive and synergistic effects that can arise from combinations of antibodies and/or nanobodies that recognize different epitopes, crystals of ternary complexes H11-H4-RBD-CR3022 (3.3Å) and H11-D4-RBD-CR3022 (2.7Å) were obtained (Table 5). The structures are similar, and we focus on the higher resolution H11-D4-RBD-CR3022 complex. As expected, the nanobody and the antibody bind to non-overlapping epitopes (FIG. 5c). Further, comparison of H11-D4-RBD-CR3022 ternary complex with both the H11-D4-RBD complex and RBD-CR3022 complex^{17,18} showed that binding of the nanobody does not perturb the recognition of the antibody and vice versa. This is consistent with biophysical analysis that shows CR3022 binds to RBD and to the nanobody RBD complex equally well (FIG. 2d, FIG. 6f).

### Discussion

It is assumed that during the virus life cycle, the Spike trimer exists in an equilibrium between the all ‘down’ configuration and mixed ‘up down’ states¹³. The Spike protein can only bind to ACE2 with the RBD in the ‘up’ state and this results in dissociation of the trimer. SARS-CoV-2 Spike binds to ACE2 with a 10 to 20-fold higher affinity (K_{D} of ˜15 nM) than SARS-CoV-1 Spike, a fact that has been proposed to drive its higher transmissibility^{13,31}. Neutralizing antibodies that have been identified to date for SARS-CoV-1 bind to the RBD of the Spike protein and many do so by blocking ACE2 binding³² but CR3022 operates by a different mechanism¹⁸. We have identified two nanobodies, H11-H4 and H11-D4, which differ in sequence at five residues within the CDR3 loop (FIG. 2a) and have shown some subtle differences in properties (FIG. 2, 3). Since the H11-H4 nanobody has the higher affinity for RBD (FIG. 2e,f), the discussion focuses on this variant but unless explicitly stated is equally valid for H11-D4.

We have shown that H11-H4 binds with high affinity to RBD (FIG. 2b,e,f), blocks ACE2 binding (FIGS. 2c, 3b,c) and neutralize the virus (FIG. 3d,e). Our analysis has suggested that H11-H4 would bind to both the ‘all down’ as well as ‘two down one up’ conformations of RBD within the Spike (FIG. 4a and FIG. 2c). The epitope on SARS-CoV-2 RBD that is recognized by H11-H4 overlaps only to a limited degree with the ACE2 binding region (FIG. 5a, b). This region of SARS-CoV-2 RBD has several sequence changes when compared to SARS-CoV-1 RBD (FIG. 1 b). The Pro469-Pro470 turn in the SARS-CoV-1 RBD structure³³ is very different to the structure at Val483-Glu484 in SARS-CoV-2. Additional sequence and structural changes between SARS-CoV-1 and SARS-CoV-2 (Tyr442->Leu455, Trp476->Phe490, Asn479->Gln493) combine to present a very different epitope and would seem to preclude cross-reactivity of H11-H4. The lack of conservation of the H11-H4 epitope between SARS-CoV-1 and SARS-CoV-2 raises the possibility SARS-CoV-2 variants may emerge that retain ACE2 receptor binding but are no longer recognized by H11-H4 or its relatives. At least some of the plausible escape mutations would perturb the position of Phe486 which inserts into a cleft in ACE2, an interaction important to SARS-CoV-2's increased affinity³⁰. The rapid pipeline from naïve library screen to maturation and thorough characterization does offer the possibility that new nanobodies could be generated against SARS-CoV-2 viruses that have escaped H11-H4.

The characterization of the cross-reactive (SARS-CoV-1 K_{D} 7 nM and SARS-CoV-2 K_{D} 40 nM) nanobody VHH72 has been reported recently^{25.} This nanobody blocks ACE2 binding and shows neutralization activity (ND₅₀ 0.2 μg/mL) against the SARS-CoV-2 pseudovirus²⁵. The crystal structure of the complex between VHH72 and RBD from SARS-CoV²⁵ showed that VHH72 recognizes an epitope that is different from that bound by H11-H4 (FIG. 14A). The epitope bound by VHH72 partly overlaps with the epitope bound by CR3022¹⁸(FIG. 14B) and is found in a crystal contact between H11-H4 and RBD in the complex (FIG. 14C and 14D). Another antibody which, like CR3022, does not block ACE2 binding but neutralizes the virus has also been published³⁴ but there are no further structural details. Humanized nanobodies with potent neutralization activity against SARS-CoV-2 virus (most potent ND₅₀ 17-36 nM in Vero cells) have been described^{35.} Some, but not all, of these nanobodies blocked ACE2 binding and no molecular insights into their mode of action were reported35. A preprint has reported a llama antibody, Ty1, that neutralizes pseudovirus and blocks ACE2 binding³⁶, but the coordinates of the EM structure were not available.

The use of convalescent serum has shown clinical promise in patients severely ill with SARS-CoV³⁷ and most recently SARS-CoV-29; such passive immune therapy has a long history in medicine³⁸. The use of laboratory produced reagents avoids some of the infection risks that arise from use of human serum and can be administered in smaller volumes. The use of antibodies as therapies is well established but nanobodies have now entered clinical trials²¹ with one, Caplacizumab²³ now licensed. The direct injection of a nanobody has also shown promise in a mouse model of cobra venom intoxication³⁹. Camelid VHH domains are highly conserved with human counter parts and their immunogenicity has been proposed to be low⁴⁰ although humanization strategies are well developed⁴¹.

To increase in vivo half-life and enhance avidity, nanobodies can be multimerized by a variety of means^{22.} For our in vitro binding assays (FIG. 3b, c) and neutralization experiments (FIG. 3d,e) we created a dimeric Fc fusion construct (FIG. 3a). Since the CR3022 antibodyl^{17,18} recognized a different epitope than H11-H4 (FIG. 2d, 5c) we investigated a combination of H11-H4 and CR3022 (CR3022 concentration fixed at 84 nM). Under these assay conditions, we observed evidence for an additive effect (FIG. 5d). Such additive combinations are a well-known strategy to reduce the propensity of the virus to escape by mutating.

This work establishes that nanobody maturation technology can be deployed to produce a highly neutralizing agent against an emerging viral threat in real time. The approach may be useful in identifying complementary epitopes to those identified by animal immunization approaches. The H11-H4 and H11-D4 nanobodies may find application in a cocktail of lab synthesized neutralizing antibodies given for passive immunization of severely ill COVID-19 patients.

### REFERENCES

1. Menachery, V. D. et al. A SARS-like cluster of circulating bat coronaviruses shows potential for human emergence. Nat Med 21, 1508-1513 (2015).
2. Ronco, C., Reis, T. & Husain-Syed, F. Management of acute kidney injury in patients with COVID-19. Lancet Resp. Med. 1-5 (2020).
3. Jaiswal, N. K. & Saxena, S. K. in Medical Virology: From Pathogenesis to Disease

Control: Coronavirus Disease 2019 (COVID-19) 141-150 (Springer Singapore, Singapore, 2020).
4. Salje, H. et al. Estimating the burden of SARS-CoV-2 in France. Science (2020).
5. Adams, M. L., Katz, D. L. & Grandpre, J. Population-Based Estimates of Chronic Conditions Affecting Risk for Complications from Coronavirus Disease, United States. Emerg Infect Dis 26, (2020).
6. Docherty, A. B. et al. Features of 20 133 UK patients in hospital with covid-19 using the ISARIC WHO Clinical Characterisation Protocol: prospective observational cohort study. BMJ 369, (2020).
7. Kucharski, A. J. et al. Early dynamics of transmission and control of COVID-19: a mathematical modelling study. Lancet Infect Dis 20, 553-558 (2020).
8. Bloch, E. M. et al. Deployment of convalescent plasma for the prevention and treatment of COVID-19. J Clin Invest 130, 2757-2765 (2020).
9. Shen, C. et al. Treatment of 5 Critically III Patients Wth COVID-19 With Convalescent Plasma. JAMA 323, 1582-1589 (2020).
10. Wan, Y., Shang, J., Graham, R., Baric, R. S. & Li, F. Receptor Recognition by the Novel Coronavirus from Wuhan: an Analysis Based on Decade-Long Structural Studies of SARS Coronavirus. J Virol 94, e00127-20 (2020).
11. Yan, R. et al. Structural basis for the recognition of SARS-CoV-2 by full-length human ACE2. Science 367, 1444-1448 (2020).
12. Li, F. Structure, Function, and Evolution of Coronavirus Spike Proteins. Annu Rev Virol 3, 237-261 (2016).
13. Wrapp, D. et al. Cryo-EM structure of the 2019-nCoV spike in the prefusion conformation. Science 367, 1260-1263 (2020).
14. Hoffmann, M. et al. SARS-CoV-2 Cell Entry Depends on ACE2 and TMPRSS2 and Is Blocked by a Clinically Proven Protease Inhibitor. Cell 181, 271-280.e8 (2020).
15. Zhu, Z. et al. Potent cross-reactive neutralization of SARS coronavirus isolates by human monoclonal antibodies. Proc Natl Acad Sci USA 104, 12123-12128 (2007).
16. Tian, X. et al. Potent binding of 2019 novel coronavirus spike protein by a SARS coronavirus-specific human monoclonal antibody. Emerg Microbes Infect 9, 382-385 (2020).
17. Yuan, M. et al. A highly conserved cryptic epitope in the receptor-binding domains of SARS-CoV-2 and SARS-CoV. Science 368, 630-633 (2020).
18. Huo, J. et al. Neutralization of SARS-CoV-2 by destruction of the prefusion Spike. Cell Host & Microbe Online, https://doi.org/10.1016/j.chom.2020.06.010 (2020).
19. Winarski, K. L. et al. Antibody-dependent enhancement of influenza disease promoted by increase in hemagglutinin stem flexibility and virus fusion kinetics. Proc Natl Acad Sci U S A 116, 15194-15199 (2019).
20. Kim, A. S., Leaman, D. P. & Zwick, M. B. Antibody to gp41 MPER alters functional properties of HIV-1 Env without complete neutralization. PLoS Pathog 10, e1004271 (2014).
21. Jovčevska, I. & Muyldermans, S. The Therapeutic Potential of Nanobodies. BioDrugs 34, 11-26 (2020).
22. Chanier, T. & Chames, P. Nanobody Engineering: Toward Next Generation Immunotherapies and Immunoimaging of Cancer. Antibodies (Basel) 8, 13 (2019).
23. Peyvandi, F. et al. Caplacizumab reduces the frequency of major thromboembolic events, exacerbations and death in patients with acquired thrombotic thrombocytopenic purpura. J Thromb Haemost 15, 1448-1452 (2017).
24. Zhou, P. et al. A pneumonia outbreak associated with a new coronavirus of probable bat origin. Nature 579, 270-273 (2020).
25. Wrapp, D. et al. Structural Basis for Potent Neutralization of Betacoronaviruses by Single-domain Camelid Antibodies. Cell 181, 1436-1441 (2020). 26. Walls, A. C. et al. Structure, Function, and Antigenicity of the SARS-CoV-2 Spike Glycoprotein. Cell 181, 281-292.e6 (2020).
27. Krissinel, E. & Henrick, K. Detection of protein assemblies in crystals. CompLife 3695, 163-174 (2005).
28. Gallivan, J. P. & Dougherty, D. A. Cation-pi interactions in structural biology. Proc Natl Acad Sci USA 96, 9459-9464 (1999).
29. Lan, J. et al. Structure of the SARS-CoV-2 spike receptor-binding domain bound to the ACE2 receptor. Nature 581, 215-220 (2020).
30. Shang, J. et al. Structural basis of receptor recognition by SARS-CoV-2. Nature 581, 221-224 (2020).
31. Song, W., Gui, M., Wang, X. & Xiang, Y. Cryo-EM structure of the SARS coronavirus spike glycoprotein in complex with its host cell receptor ACE2. PLoS Pathog 14, e1007236 (2018).
32. Suits, M. D., Sperandeo, P., Dehò, G., Polissi, A. & Jia, Z. Novel structure of the conserved gram-negative lipopolysaccharide transport protein A and mutagenesis analysis. J Mol Biol 380, 476-488 (2008).
33. Li, F., Li, W., Farzan, M. & Harrison, S. C. Structure of SARS coronavirus spike receptor-binding domain complexed with receptor. Science 309, 1864-1868 (2005).
34. Wang, C. et al. A human monoclonal 1 antibody blocking SARS-CoV-2 infection. Nature Comms 11, 2251 (2020).
35. Chi, X. et al. Humanized Single Domain Antibodies Neutralize SARS-CoV-2 by Targeting Spike Receptor Binding Domain. bioRxiv (2020).
36. Hanke, L. et al. An alpaca nanobody neutralizes SARS-CoV-2 by blocking receptor interaction. Biochem Biophys Acta BioRxiv, (2020).
37. Mair-Jenkins, J. et al. The effectiveness of convalescent plasma and hyperimmune immunoglobulin for the treatment of severe acute respiratory infections of viral etiology: a systematic review and exploratory meta-analysis. J Infect Dis 211, 80-90 (2015).
38. Slifka, M. K. & Amanna, I. J. Passive immunization. Plotkin's Vaccines, 7th Edition 8, 84-95 (2018).
39. Richard, G. et al. In vivo neutralization of a-cobratoxin with high-affinity llama single-domain antibodies (VHHs) and a VHH-Fc antibody. PLoS One 8, e69495 (2013).
40. Klarenbeek, A. et al. Camelid Ig V genes reveal significant human homology not seen in therapeutic target genes, providing for a powerful therapeutic antibody platform. MAbs 7, 693-706 (2015).
41. Vincke, C. et al. General strategy to humanize a camelid single-domain antibody and identification of a universal humanized nanobody scaffold. J Biol Chem 284, 3273-3284 (2009).
42. Laskowski, R. A. & Swindells, M. B. LigPlot+: multiple ligand-protein interaction diagrams for drug discovery. J Chem Inf Model 51, 2778-2786 (2011). Caly, L. et al. Isolation and rapid sharing of the 2019 novel coronavirus (SARS-CoV-2) from the first patient diagnosed with COVID-19 in Australia. Med J Aust 212, 459-462 (2020).
44. Johnson, R. M., Dahlgren, L., Siegfried, B. D. & Ellis, M. D. Acaricide, fungicide and drug interactions in honey bees (Apis mellifera). PLoS One 8, e54092 (2013).
45. de Madrid, A. T. & Porterfield, J. S. A simple micro-culture method for the study of group B arboviruses. Bulletin of the World Health Organization 40, 113 (1969).
46. Zivanov, J. et al. New tools for automated high-resolution cryo-EM structure determination in RELION-3. Elife 7, (2018).
47. Punjani, A., Rubinstein, J. L., Fleet, D. J. & Brubaker, M. A. cryoSPARC: algorithms for rapid unsupervised cryo-EM structure determination. Nat Methods 14, 290-296 (2017).
48. Asarnow, D., Palovcak, E. & Cheng, Y. UCSF pyem v0.5. Zenodo. https://doi.org/10.5281/zenodo.3576630 (2019).
49. Pettersen, E. F. et al. UCSF chimera - A visualization system for exploratory research and analysis. J. Comp. I Chem, 25, 1605-1612 (2004).
50. Emsley, P. & Cowtan, K. Coot: model-building tools for molecular graphics. Acta Crystallogr D Struct Biol 60, 2126-2132 (2004).
51. Liebschner, D. et al. Macromolecular structure determination using X-rays, neutrons and electrons: recent developments in Phenix. Acta Crystallogr D Struct Biol 75, 861-877 (2019).
52. Wagner, T. et al. SPHIRE-crYOLO is a fast and accurate fully automated particle picker for cryo-EM. Commun Biol 2, 218 (2019).
53. Murshudov, G. N. et al. REFMACS for the refinement of macromolecular crystal structures. Acta Crystallogr D Biol Crystallogr 67, 355-367 (2011).
54. Burnley, T., Palmer, C. M. & Winn, M. Recent developments in the CCP-EM software suite. Acta Crystallogr D Struct Biol 73, 469-477 (2017).
55. Winter, G., Lobley, C. M. & Prince, S. M. Decision making in xia2. Acta Crystallogr D Biol Crystallogr 69, 1260-1273 (2013).
56. Winter, G. xia2: an expert system for macromolecular crystallography data reduction. J Appl Crystallogr 43, 186-190 (2009).
57. Schmitz, K. R., Bagchi, A., Roovers, R. C., van Bergen en Henegouwen, P. M. & Ferguson, K. M. Structural evaluation of EGFR inhibition mechanisms for nanobodies/VHH domains. Structure 21, 1214-1224 (2013).

58. Kovalevskiy, O., Nicholls, R. A., Long, F., Carlon, A. & Murshudov, G. N. Overview of refinement procedures within REFMACS: utilizing data from different sources. Acta Crystallogr D Struct Biol 74, 215-227 (2018).
59. Joosten, R. P., Long, F., Murshudov, G. N. & Perrakis, A. The PDB_REDO server for macromolecular structure model optimization. IUCrJ 1, 213-220 (2014).
60. Davis, I. W. et al. MolProbity: all-atom contacts and structure validation for proteins and nucleic acids. Nucleic Acids Res 35, W375-83 (2007).
61. Painter, J. & Merritt, E. A. TLSMD web server for the generation of multi-group TLS models. J Appl Crystallogr. 39, 109-111 (2006).
62. McCoy, A. J. et al. Phaser crystallographic software. J Appl Crystallogr 40, 658-674 (2007).
63. Jakobi, A. J., Wilmanns, M. & Sachse, C. Model-based local density sharpening of cryo-EM maps. Elife 6, (2017).

1-26. (canceled)

27. An anti-SARS-CoV-2 single domain antibody comprising:
a CDR1 comprising SEQ ID NO:1, a CDR2 comprising SEQ ID NO:2 and a CDR3 comprising an amino acid sequence selected from SEQ ID NOs: 40, 41, 42, 43, 44, 45, 46, 47, 48, 49, 50, 51, 52, 53, 54, 55, 56, 57, 58, 59 and 60, wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications, and wherein the anti-SARS-CoV-2 single domain antibody
(a) blocks, modulates or inhibits the binding between the receptor binding domain of SARS CoV 2 Spike protein and the angiotensin converting enzyme 2 receptor (ACE2),
(b) has a Kd value for SARS-CoV-2 Spike protein of less than 20 nM and
(c) and has an ND50 value for SARS-CoV-2 of less than 100 nM .

28. The anti-SARS-CoV-2 single domain antibody to claim 27, wherein the amino acid modification is a substitution, insertion or deletion.

29. The anti-SARS-CoV-2 single domain antibody according to claim 27 comprising:
a CDR3 comprising an amino acid sequence selected from SEQ ID NOs: 40, 41, 42, 43 and 44.

30. The anti-SARS-CoV-2 single domain antibody of claim 27, wherein the single domain antibody further comprises four framework regions (FR1, FR2, FR3 and FR4).

31. The anti-SARS-CoV-2 single domain antibody of claim 27 comprising an amino acid sequence having at least 70% identity to a sequence selected from the group consisting of: SEQ 87, 88, 89, 90, 91, 92, 93, 94, 95, 96, 97, 98, 99, 101, 102, 103, 104, 105, 106 and 107.

32. A polynucleotide encoding a single domain antibody according to claim 27.

33. An expression vector comprising the polynucleotide of claim 32.

34. A host cell or cell line comprising the vector according to claim 33.

35. A method for the production of a single domain antibody, comprising culturing a host cell according to claim 34 in a culture medium under conditions to express the polynucleotide sequence of the expression vector.

36. A pharmaceutical composition comprising a single domain antibody according to claim 27.

37. A single domain antibody according to claim 27 for use in medicine, optionally wherein the coronavirus is SARS-CoV-2.

38. A single domain antibody according to claim 27 for use in the treatment or prophylaxis of a coronavirus infection, optionally wherein the coronavirus is SARS-CoV-2.

39. A method for the treatment or prevention of coronavirus, said method comprising administering to a subject a therapeutically active amount of a single domain antibody according to claim 27, optionally wherein the coronavirus is SARS-CoV-2.

40. Use of a single domain antibody according to claim 27 in the manufacture of a medicament for use in the treatment of a coronavirus.

41. A pharmaceutical device comprising a single domain antibody according to claim 27, wherein the pharmaceutical device is suitable for delivery of the single domain antibody via inhalation.

42. A method for diagnosing coronavirus infection in a subject, the method comprising:
(a) contacting an isolated sample with the single domain antibody of claim 27,
(b) detecting the number of antibody-antigen complexes,
(c) detecting the presence of coronavirus in the sample,
wherein the presence of the complex provides a positive diagnosis of coronavirus in the subject, optionally wherein the coronavirus is SARS-CoV-2.

43. A method for detecting coronavirus protein in a subject, wherein the method comprises the steps of
(a) obtaining a sample from a subject,
(b) contacting a sample from the subject with a single domain antibody of claim 27, and
(c) detecting the antibody-antigen complex,
wherein the presence of the complex indicates the presence of coronavirus protein in the subject, optionally wherein the coronavirus is SARS-CoV--2.

44. A method for detecting coronavirus protein in a sample from a patient, wherein the method comprises the steps of
(a) contacting a sample from the subject with a single domain antibody of claim 27, and
(b) detecting the antibody-antigen complex,
wherein the presence of the complex indicates the presence of coronavirus protein in the subject, optionally wherein the coronavirus is SARS-CoV-2.

45. An assay for the detection of a coronavirus, wherein the assay comprises contacting a sample obtained from a patient with a single domain antibody of claim 27, wherein the single domain antibody comprises a detectable label or reporter molecule to selectively isolate the coronavirus in the patient sample, optionally wherein the assay is an enzyme-linked immunosorbent assay (ELISA), a radioimmunoassay (RIA) or a fluorescence-activated cell sorting (FACS) .

46. A kit for the detection of a coronavirus, wherein the kit comprises:
(a) a detectable marker
(b) a single domain antibody of claim 27.

47. A single domain antibody according to a pharmaceutical composition according to claim 36 for use in medicine, optionally wherein the coronavirus is SARS-CoV-2.

48. A single domain antibody according to a pharmaceutical composition according to claim 36 for use in the treatment or prophylaxis of a coronavirus infection, optionally wherein the coronavirus is SARS-CoV-2.


SEQUENCE LISTING (128 sequences)

SEQ ID NO: 1 - protein, 8 aa, Artificial Sequence
  NbRBD_H11 CDR1
GRTFSTAA

SEQ ID NO: 2 - protein, 8 aa, Artificial Sequence
  NbRBD_H11 CDR2
IRWSGGSA

SEQ ID NO: 3 - protein, 20 aa, Artificial Sequence
  NbRBD_H11 CDR3
AQTRVTRSLLSDYATWPYDY

SEQ ID NO: 4 - protein, 8 aa, Artificial Sequence
  NbRBD_A7 CDR1
GFTFDDYA

SEQ ID NO: 5 - protein, 8 aa, Artificial Sequence
  NbRBD_A7 CDR2
ISWNGGGT

SEQ ID NO: 6 - protein, 20 aa, Artificial Sequence
  NbRBD_A7 CDR3
AQTRVTRSLLSDYATWPYDY

SEQ ID NO: 7 - protein, 8 aa, Artificial Sequence
  NbRBD_F9 CDR1
GFTFSSYF

SEQ ID NO: 8 - protein, 7 aa, Artificial Sequence
  NbRBD_F9 CDR2
IGDRGNT

SEQ ID NO: 9 - protein, 10 aa, Artificial Sequence
  NbRBD_F9 CDR3
YSRNRILQHY

SEQ ID NO: 10 - protein, 8 aa, Artificial Sequence
  NbRBD_C10 CDR1
GFAFSSYD

SEQ ID NO: 11 - protein, 8 aa, Artificial Sequence
  NbRBD_C10 CDR2
IDSGGGVT

SEQ ID NO: 12 - protein, 13 aa, Artificial Sequence
  NbRBD_C10 CDR3
NAVDGLGYLEYDY

SEQ ID NO: 13 - protein, 8 aa, Artificial Sequence
  NbRBD_B11 CDR1
GFTFDDYA

SEQ ID NO: 14 - protein, 8 aa, Artificial Sequence
  NbRBD_B11 CDR2
IYSYISTT

SEQ ID NO: 15 - protein, 12 aa, Artificial Sequence
  NbRBD_B11 CDR3
AARRLDTTDYKY

SEQ ID NO: 16 - protein, 8 aa, Artificial Sequence
  NbRDB_E11 CDR1
GFTFSNYA

SEQ ID NO: 17 - protein, 8 aa, Artificial Sequence
  NbRDB_E11 CDR2
IGSDGRHP

SEQ ID NO: 18 - protein, 13 aa, Artificial Sequence
  NbRDB_E11 CDR3
LPGWSTVGTFLDA

SEQ ID NO: 19 - protein, 8 aa, Artificial Sequence
  NbRBD_D1 CDR1
GFTFSSYG

SEQ ID NO: 20 - protein, 8 aa, Artificial Sequence
  NbRBD_D1 CDR2
INSGGGST

SEQ ID NO: 21 - protein, 16 aa, Artificial Sequence
  NbRBD_D1 CDR3
AKDASEDAGRLYWTDY

SEQ ID NO: 22 - protein, 8 aa, Artificial Sequence
  NbRBD_G7 CDR1
GFTFDTYD

SEQ ID NO: 23 - protein, 7 aa, Artificial Sequence
  NbRBD_G7 CDR2
EDSTGNK

SEQ ID NO: 24 - protein, 9 aa, Artificial Sequence
  NbRBD_G7 CDR3
AKGLRSWGA

SEQ ID NO: 25 - protein, 8 aa, Artificial Sequence
  NbRBD_F5 CDR1
GTIFRINA

SEQ ID NO: 26 - protein, 8 aa, Artificial Sequence
  NbRBD_F5 CDR2
ISNGDSGD

SEQ ID NO: 27 - protein, 12 aa, Artificial Sequence
  NbRBD_F5 CDR3
YCNVEASWPHKY

SEQ ID NO: 28 - protein, 8 aa, Artificial Sequence
  NbRBD_G11 CDR1
GFTFDDYG

SEQ ID NO: 29 - protein, 7 aa, Artificial Sequence
  NbRBD_G11 CDR2
VNSGGGT

SEQ ID NO: 30 - protein, 14 aa, Artificial Sequence
  NbRBD_G11 CDR3
AKRDGSWWGYTTDY

SEQ ID NO: 31 - protein, 8 aa, Artificial Sequence
  NbRBD_B4 CDR1
GFTFSSYW

SEQ ID NO: 32 - protein, 8 aa, Artificial Sequence
  NbRBD_B4 CDR2
INTGGDTT

SEQ ID NO: 33 - protein, 14 aa, Artificial Sequence
  NbRBD_B4 CDR3
ARTGVVRGPGDLDA

SEQ ID NO: 34 - protein, 8 aa, Artificial Sequence
  NbRBD_G9 CDR1
GFTFYDYH

SEQ ID NO: 35 - protein, 8 aa, Artificial Sequence
  NbRBD_G9 CDR2
IDTGGGRT

SEQ ID NO: 36 - protein, 13 aa, Artificial Sequence
  NbRBD_G9 CDR3
VRDGDARGPDYDY

SEQ ID NO: 37 - protein, 8 aa, Artificial Sequence
  NbRBD_C7 CDR1
GFTFSSYD

SEQ ID NO: 38 - protein, 8 aa, Artificial Sequence
  NbRBD_C7 CDR2
ISGSDSTT

SEQ ID NO: 39 - protein, 9 aa, Artificial Sequence
  NbRBD_C7 CDR3
ARPLGQYTS

SEQ ID NO: 40 - protein, 20 aa, Artificial Sequence
  NbRBD_H11-D4 CDR3
ARTENVRSLLSDYATWPYDY

SEQ ID NO: 41 - protein, 20 aa, Artificial Sequence
  NbRBD_H11-H4 CDR3
AQTHYVSYLLSDYATWPYDY

SEQ ID NO: 42 - protein, 20 aa, Artificial Sequence
  NbRBD_H11-H6 CDR3
AGSKITRSLLSDYATWPYDY

SEQ ID NO: 43 - protein, 20 aa, Artificial Sequence
  NbRBD_H11-A10 CDR3
AGFSATRSLLSDYATWPYDY

SEQ ID NO: 44 - protein, 20 aa, Artificial Sequence
  NbRBD_H11-B5 CDR3
ASYQATRSLLSDYATWPYDY

SEQ ID NO: 45 - protein, 20 aa, Artificial Sequence
  NbRBD_H11-A7 CDR3
AQTDNVRALLSDYATWPYDY

SEQ ID NO: 46 - protein, 20 aa, Artificial Sequence
  NbRBD_H11-F7 CDR3
AIFSNVRSLLSDYATWPYDY

SEQ ID NO: 47 - protein, 20 aa, Artificial Sequence
  NbRBD_H11-F6 CDR3
ARTSNVRSLLSDYATWPYDY

SEQ ID NO: 48 - protein, 20 aa, Artificial Sequence
  NbRBD_H11-G8 CDR3
AGSGVTRSLLSDYATWPYDY

SEQ ID NO: 49 - protein, 20 aa, Artificial Sequence
  NbRBD_H11-D1 CDR3
AGWRATRSLLSDYATWPYDY

SEQ ID NO: 50 - protein, 20 aa, Artificial Sequence
  NbRBD_H11-A9 CDR3
APYEATRSLLSDYATWPYDY

SEQ ID NO: 51 - protein, 20 aa, Artificial Sequence
  NbRBD_H11-C6 CDR3
AQTGYTSWLLSDYATWPYDY

SEQ ID NO: 52 - protein, 20 aa, Artificial Sequence
  NbRBD_H11-E3 CDR3
AVYHATRSLLSDYATWPYDY

SEQ ID NO: 53 - protein, 20 aa, Artificial Sequence
  NbRBD_H11-F4 CDR3
AESTITRSLLSDYATWPYDY

SEQ ID NO: 54 - protein, 20 aa, Artificial Sequence
  NbRBD_H11-C5 CDR3
AQTSYVSFLLSDYATWPYDY

SEQ ID NO: 55 - protein, 20 aa, Artificial Sequence
  NbRBD_H11-C2 CDR3
AGSRATRSLLSDYATWPYDY

SEQ ID NO: 56 - protein, 20 aa, Artificial Sequence
  NbRBD_H11-B11 CDR3
AETLNTRSLLSDYATWPYDY

SEQ ID NO: 57 - protein, 20 aa, Artificial Sequence
  NbRBD_H11-A3 CDR3
ARSDNVRSLLSDYATWPYDY

SEQ ID NO: 58 - protein, 20 aa, Artificial Sequence
  NbRBD_H11-D12 CDR3
ADWGVTRSLLSDYATWPYDY

SEQ ID NO: 59 - protein, 20 aa, Artificial Sequence
  NbRBD_H11-D6 CDR3
ASSSVTRSLLSDYATWPYDY

SEQ ID NO: 60 - protein, 20 aa, Artificial Sequence
  NbRBD_H11-F8 CDR3
ADASATRSLLSDYATWPYDY

SEQ ID NO: 61 - protein, 127 aa, Artificial Sequence
  NbRBD_H11
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTRVTRSLLSDYATWPYDYWGQGTQVTVSS

SEQ ID NO: 62 - protein, 123 aa, Artificial Sequence
  NbRBD_A7
QVQLVESGGGLVQPGGSLRLSCAASGFTFDDYAMSWVRQAPGKGLEWVSAISWNGGGTYYAESMKGRFTISRDNAKNTLYLQMNSLKSEDTAVYYCAKDFHRYGSSTLEYDYWGQGTQVTVSS

SEQ ID NO: 63 - protein, 116 aa, Artificial Sequence
  NbRBD_F9
QVQLVESGGGLVQPGGSLRVSCAASGFTFSSYFMSWVRQAPGKQRELVATIGDRGNTNYADSVKGRFTISRDNAKNTVYLQMNSLKPEDTAVYYCYSRNRILQHYWGQGTQVTVSS

SEQ ID NO: 64 - protein, 120 aa, Artificial Sequence
  NbRBD_C10
QVQLVESGGGLVQPGGSLRLSCAASGFAFSSYDMSWVRQAPGKGLEWVSAIDSGGGVTYYADSMKGRFTISRDNAKNTVYLQMNSLKPEDTAVYYCNAVDGLGYLEYDYWGQGTQVTVSS

SEQ ID NO: 65 - protein, 119 aa, Artificial Sequence
  NbRBD_B11
QVQLVESGGGLVQPGGSLRLSCAASGFTFDDYAMSWVRQAPGKGLEWVSNIYSYISTTHYADSVKGRFTISTDNAKNTLYLQMNSLKPEDTAVYYCAARRLDTTDYKYWGQGTQVTVSS

SEQ ID NO: 66 - protein, 120 aa, Artificial Sequence
  NbRDB_E11
QVQLVESGGGLVQPGGSLRLSCAASGFTFSNYAMTWVRQAPGKGLEWISDIGSDGRHPAYADSVKGRYTISRDNAKNTLYLQMNSLKPEDTAMYYCLPGWSTVGTFLDAWGQGTQVTVSS

SEQ ID NO: 67 - protein, 123 aa, Artificial Sequence
  NbRBD_D1
QVQLVESGGGLVQPGGSLRLSCAASGFTFSSYGMSWVRQAPGKGLEWVSGINSGGGSTSYADSVKGRFTISRDNAKNTLYLQMNSLKSEDTAVYYCAKDASEDAGRLYWTDYWGQGTQVTVSS

SEQ ID NO: 68 - protein, 115 aa, Artificial Sequence
  NbRBD_G7
QVQLVESGGGLVQPGGSLRLSCATSGFTFDTYDMSWVRQAPGNGPEWVSAEDSTGNKFYADSVKGRFAISRDNARNTLYLQMNSLTSEDTAVYYCAKGLRSWGAWGQGTQVTVSS

SEQ ID NO: 69 - protein, 119 aa, Artificial Sequence
  NbRBD_F5
QVQLVESGGGLVQAGGSLRLSCAASGTIFRINAMAWYRQAPGKQRELVAIISNGDSGDSTNYADSVKGRFTISRDSAKNTVYLQMNSLKPEDTAVYYCNVEASWPHKYWGQGTQVTVSS

SEQ ID NO: 70 - protein, 120 aa, Artificial Sequence
  NbRBD_G11
QVHLVESGGGLVQPGGSLRLSCAASGFTFDDYGMTWVRQAPGKGLEWVSAVNSGGGTSYADSVKGRFTISRDNAKNTLYLQMNSLKPEDTAVYYCAKRDGSWWGYTTDYWGKGTQVTVSS

SEQ ID NO: 71 - protein, 121 aa, Artificial Sequence
  NbRBD_B4
QVQLVESGGGLVQPGGSLRLSCAASGFTFSSYWMYWVRQAPGKGLEWVSVINTGGDTTYYADSVKGRFTISRDNDENTLSLQMNSLKPEDTALYYCARTGVVRGPGDLDAWGQGTQVTVSS

SEQ ID NO: 72 - protein, 120 aa, Artificial Sequence
  NbRBD_G9
QVQLVESGGGLVQPGGSLRLSCAASGFTFYDYHMSWVRQAPGKGLEWVSGIDTGGGRTYAADSVKGRFTISRDNAKNTLYLQMNSLKSEDTALYYCVRDGDARGPDYDYWGQGTMVTVSS

SEQ ID NO: 73 - protein, 116 aa, Artificial Sequence
  NbRBD_C7
QVQLVESGGGLVQPGGSLRLSCAASGFTFSSYDMSWVRQAPGKGLEWVSGISGSDSTTVYSDSVKDRFTISRDNAKNTLYLQMNKLKPEDTALYYCARPLGQYTSWGQGTQVTVSS

SEQ ID NO: 74 - DNA, 381 bp, Artificial Sequence
  NbRBD_H11
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacgcgggtcacgcggtccctccttagcgactatgccacttggccttatgactactggggccaggggacccaggtcaccgtctcctca

SEQ ID NO: 75 - DNA, 369 bp, Artificial Sequence
  NbRBD_A7
caggtgcagctggtagagtctgggggaggcttggtgcagcctggggggtctctgagactctcctgtgcagcctctggattcacttttgatgattatgccatgagctgggtccgacaggctccagggaaggggctggagtgggtctcagctattagctggaatggtggtggcacatactatgcagaatccatgaagggccgattcaccatctccagagacaacgccaagaacacgctgtatctgcaaatgaacagtctgaaatctgaggacacggccgtgtattactgtgcaaaagattttcatcgatacggtagtagcactttggagtatgactactggggccaggggacccaggtcaccgtctcctca

SEQ ID NO: 76 - DNA, 348 bp, Artificial Sequence
  NbRBD_F9
caggtgcagctggtagagtctgggggaggcttggtgcagcctggggggtctctgagagtctcctgtgcagcctctggattcaccttcagtagctacttcatgagctgggtccgccaggctccagggaagcagcgcgagttggtcgcgactattggtgatcgtggtaacacaaactatgcagactccgtgaagggccgattcaccatctccagagacaacgccaagaacacagtgtatctgcaaatgaacagcctgaaacctgaggacacggccgtctattactgttattcaagaaaccgtatcttgcaacactactggggccaggggacacaggtcaccgtctcctca

SEQ ID NO: 77 - DNA, 360 bp, Artificial Sequence
  NbRBD_C10
caggtgcagctggtagagtctgggggaggcttggtgcagcctggggggtctctgagactctcctgtgcagcctctggattcgccttcagtagctatgacatgagctgggtccgccaggctccagggaagggactcgaatgggtctcagcgattgatagtggtggtggtgtcacatactatgcagactccatgaagggccgattcaccatctccagagacaacgccaagaacacggtgtatctgcaaatgaacagcctgaaacctgaggacacggccgtgtattactgtaatgcagtagacggcttggggtacttagagtatgactactggggccaggggacccaggtcaccgtctcctca

SEQ ID NO: 78 - DNA, 357 bp, Artificial Sequence
  NbRBD_B11
caggtgcagctggtagagtctgggggaggcttggtacagcctggggggtctctgagactctcctgtgcagcctctggattcacttttgatgattatgccatgagctgggtccgacaggctccagggaaggggctggagtgggtgtccaatatttatagttacattagtaccacacactatgcagactccgtgaagggccgattcaccatctccacagacaacgccaagaacacactgtatctgcaaatgaacagcctgaaacctgaggacacggccgtttattactgtgcagcccgaagacttgacacgaccgactataaatactggggccaggggacacaggtcaccgtctcctca

SEQ ID NO: 79 - DNA, 360 bp, Artificial Sequence
  NbRDB_E11
caggtgcagctggtagagtctgggggaggcttggtgcagcctggggggtctctgagactctcctgtgcagcctctggattcaccttcagtaactatgccatgacctgggtccgccaggctccaggaaaggggctcgagtggatctcagatattggtagtgatggtaggcacccagcctatgcagactccgtgaagggccgatacaccatctccagagacaacgccaagaacacgctatatctgcaaatgaacagcctgaaacctgaggacacggccatgtattactgtctcccgggttggagtaccgtcgggacttttttggacgcatggggccaggggacccaggtcaccgtctcctca

SEQ ID NO: 80 - DNA, 369 bp, Artificial Sequence
  NbRBD_D1
caggtgcagctggtagagtctgggggaggcttggtgcagcctggggggtctctgagactctcctgtgcagcctctggattcaccttcagtagctatggcatgagctgggtccgccaggctccaggaaaggggctcgagtgggtctcaggtattaatagtggtggtggtagcacaagctatgcagactccgtgaagggccgattcaccatctccagagacaacgccaagaacacgctgtatctgcaaatgaacagtctgaaatctgaggacacggccgtgtattactgtgcaaaagacgctagtgaagatgcggggcgactatactggaccgactactggggccaggggacccaggtcaccgtctcctca

SEQ ID NO: 81 - DNA, 345 bp, Artificial Sequence
  NbRBD_G7
caggtgcagctggtggagtctgggggaggcttggtgcagcctgggggttctctgagactctcctgtgcaacctctggattcaccttcgatacgtatgacatgagctgggtccgccaggctccaggaaacgggcccgagtgggtctcagcggaggatagtactgggaacaaattctatgcagactctgtgaagggccgattcgccatctccagagacaacgccaggaacacactatatctgcaaatgaacagtctgacatctgaagacacggccgtgtattactgtgcaaaaggtctacgatcctggggtgcctggggccaggggacccaggtcaccgtctcctca

SEQ ID NO: 82 - DNA, 357 bp, Artificial Sequence
  NbRBD_F5
caggtgcagctggtagagtctgggggaggcttggtgcaggctggggggtctctgagactctcctgtgcagcctctggaaccatcttcagaatcaatgccatggcctggtaccgccaggctccagggaagcagcgcgagttggtcgcaattatttctaatggtgatagtggtgatagtacaaactatgcagactccgtgaagggccgattcaccatctccagagacagcgccaagaacacggtgtatctgcaaatgaacagcctgaaacctgaggacacagccgtctattactgtaatgtagaagcgtcctggccccataagtactggggccaggggacccaggtcaccgtctcctca

SEQ ID NO: 83 - DNA, 360 bp, Artificial Sequence
  NbRBD_G11
caggtgcacctggtagagtctgggggaggcttggtgcagcctggggggtctctgagactctcctgtgcagcctctggattcacttttgatgattatggcatgacctgggtccgccaggctccaggaaaggggctcgagtgggtctcagcggttaatagtggtggtggtacaagctatgcagactccgtgaagggccgattcaccatctccagagacaacgccaagaacacgctgtatctgcaaatgaacagcctgaaacctgaggacacggccgtgtattactgtgcaaaaagagatggtagttggtggggttataccacggactactggggcaaagggacccaggtcaccgtctcctca

SEQ ID NO: 84 - DNA, 363 bp, Artificial Sequence
  NbRBD_B4
caggtgcagctggtggagtctgggggaggcttggtgcagcctggggggtctctgagactctcctgtgcagcctctggattcaccttcagtagctactggatgtattgggtccgtcaggctccagggaaggggctcgagtgggtctcagtaattaatactggtggtgatactacatactatgcagactccgtgaagggccgattcaccatctccagagacaacgacgagaacacgctgtctctgcaaatgaacagcctgaaacctgaggacacggccctgtattactgtgcgagaacaggggtagtacgtggtccgggcgatttggacgcatggggccaggggacccaggtcaccgtctcctca

SEQ ID NO: 85 - DNA, 360 bp, Artificial Sequence
  NbRBD_G9
caggtgcagctggtggagtctgggggaggcttggtgcagcctggggggtctctgagactctcctgtgcagcctctggattcactttttatgattatcacatgagctgggtccgccaggctccagggaaggggctcgagtgggtctcaggtatcgatactggtggtgggcgcacatacgctgcagactccgtgaagggccgattcaccatctccagagacaacgccaagaacacactgtatctgcaaatgaacagcctgaaatctgaggacacggccctgtattattgtgtgagagatggagatgcacgggggcccgactatgactactggggccaggggaccatggtcaccgtctcctca

SEQ ID NO: 86 - DNA, 348 bp, Artificial Sequence
  NbRBD_C7
caggtgcagctggtagagtctgggggaggcttggtgcagcctggggggtctctgagactctcctgtgcagcctctggattcaccttcagtagctatgacatgagctgggtccgccaggctccaggaaaggggctcgagtgggtctcaggtattagcggaagtgatagtactacagtctattcagactccgtgaaggaccgattcaccatctccagagacaacgccaagaatacgttgtatctgcaaatgaacaaactgaaacctgaggacacggccctgtattactgtgcaagaccattgggacaatacacgagttggggccaggggacccaggtcaccgtctcctca

SEQ ID NO: 87 - protein, 127 aa, Artificial Sequence
  NbRBD_H11-D4
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSS

SEQ ID NO: 88 - protein, 127 aa, Artificial Sequence
  NbRBD_H11-H4
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSS

SEQ ID NO: 89 - protein, 124 aa, Artificial Sequence
  NbRBD_H11-H6
LVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGSKITRSLLSDYATWPYDYWGQGTQVTVSS

SEQ ID NO: 90 - protein, 127 aa, Artificial Sequence
  NbRBD_H11-A10
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGFSATRSLLSDYATWPYDYWGQGTQVTVSS

SEQ ID NO: 91 - protein, 127 aa, Artificial Sequence
  NbRBD_H11-B5
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCASYQATRSLLSDYATWPYDYWGQGTQVTVSS

SEQ ID NO: 92 - protein, 127 aa, Artificial Sequence
  NbRBD_H11-A7
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTDNVRALLSDYATWPYDYWGQGTQVTVSS

SEQ ID NO: 93 - protein, 127 aa, Artificial Sequence
  NbRBD_H11-F7
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAIFSNVRSLLSDYATWPYDYWGQGTQVTVSS

SEQ ID NO: 94 - protein, 127 aa, Artificial Sequence
  NbRBD_H11-F6
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTSNVRSLLSDYATWPYDYWGQGTQVTVSS

SEQ ID NO: 95 - protein, 127 aa, Artificial Sequence
  NbRBD_H11-G8
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGSGVTRSLLSDYATWPYDYWGQGTQVTVSS

SEQ ID NO: 96 - protein, 127 aa, Artificial Sequence
  NbRBD_H11-D1
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGWRATRSLLSDYATWPYDYWGQGTQVTVSS

SEQ ID NO: 97 - protein, 127 aa, Artificial Sequence
  NbRBD_H11-A9
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAPYEATRSLLSDYATWPYDYWGQGTQVTVSS

SEQ ID NO: 98 - protein, 127 aa, Artificial Sequence
  NbRBD_H11-C6
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTGYTSWLLSDYATWPYDYWGQGTQVTVSS

SEQ ID NO: 99 - protein, 127 aa, Artificial Sequence
  NbRBD_H11-E3
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAVYHATRSLLSDYATWPYDYWGQGTQVTVSS

SEQ ID NO: 100 - protein, 127 aa, Artificial Sequence
  NbRBD_H11-F4
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAESTITRSLLSDYATWPYDYWGQGTQVTVSS

SEQ ID NO: 101 - protein, 127 aa, Artificial Sequence
  NbRBD_H11-C5
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTSYVSFLLSDYATWPYDYWGQGTQVTVSS

SEQ ID NO: 102 - protein, 127 aa, Artificial Sequence
  NbRBD_H11-C2
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGSRATRSLLSDYATWPYDYWGQGTQVTVSS

SEQ ID NO: 103 - protein, 127 aa, Artificial Sequence
  NbRBD_H11-B11
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAETLNTRSLLSDYATWPYDYWGQGTQVTVSS

SEQ ID NO: 104 - protein, 127 aa, Artificial Sequence
  NbRBD_H11-A3
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARSDNVRSLLSDYATWPYDYWGQGTQVTVSS

SEQ ID NO: 105 - protein, 127 aa, Artificial Sequence
  NbRBD_H11-D12
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCADWGVTRSLLSDYATWPYDYWGQGTQVTVSS

SEQ ID NO: 106 - protein, 127 aa, Artificial Sequence
  NbRBD_H11-D6
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCASSSVTRSLLSDYATWPYDYWGQGTQVTVSS

SEQ ID NO: 107 - protein, 127 aa, Artificial Sequence
  NbRBD_H11-F8
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCADASATRSLLSDYATWPYDYWGQGTQVTVSS

SEQ ID NO: 108 - DNA, 381 bp, Artificial Sequence
  NbRBD_H11-D4
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacggacggagaatgttcggtccctccttagcgactatgccacttggccttatgactactggggccaggggacccaggtcaccgtctcctca

SEQ ID NO: 109 - DNA, 381 bp, Artificial Sequence
  NbRBD_H11-H4
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacgcattatgtttcttatctccttagcgactatgccacttggccttatgactactggggccaggggacccaggtcaccgtctcctca

SEQ ID NO: 110 - DNA, 381 bp, Artificial Sequence
  NbRBD_H11-H6
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaggttctaagattacgcggtccctccttagcgactatgccacttggccttatgactactggggccaggggacccaggtcaccgtctcctca

SEQ ID NO: 111 - DNA, 381 bp, Artificial Sequence
  NbRBD_H11-A10
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaggttttagtgcgactcggtccctccttagcgactatgccacttggccttatgactactggggccaggggacccaggtcaccgtctcctca

SEQ ID NO: 112 - DNA, 381 bp, Artificial Sequence
  NbRBD_H11-B5
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaagttatcaggcgactcggtccctccttagcgactatgccacttggccttatgactactggggccaggggacccaggtcaccgtctcctca

SEQ ID NO: 113 - DNA, 381 bp, Artificial Sequence
  NbRBD_H11-A7
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacggataatgtgcgggcgctccttagcgactatgccacttggccttatgactactggggccaggggacccaggtcaccgtctcctca

SEQ ID NO: 114 - DNA, 381 bp, Artificial Sequence
  NbRBD_H11-F7
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaattttttctaatgtgcggtccctccttagcgactatgccacttggccttatgactactggggccaggggacccaggtcaccgtctcctca

SEQ ID NO: 115 - DNA, 381 bp, Artificial Sequence
  NbRBD_H11-F6
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacggacgtcgaatgtgcggtccctccttagcgactatgccacttggccttatgactactggggccaggggacccaggtcaccgtctcctca

SEQ ID NO: 116 - DNA, 381 bp, Artificial Sequence
  NbRBD_H11-G8
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagggtctggtgtgacgcggtccctccttagcgactatgccacttggccttatgactactggggccaggggacccaggtcaccgtctcctca

SEQ ID NO: 117 - DNA, 381 bp, Artificial Sequence
  NbRBD_H11-D1
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagggtggcgtgcgacgcggtccctccttagcgactatgccacttggccttatgactactggggccaggggacccaggtcaccgtctcctca

SEQ ID NO: 118 - DNA, 381 bp, Artificial Sequence
  NbRBD_H11-A9
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaccgtatgaggctacgcggtccctccttagcgactatgccacttggccttatgactactggggccaggggacccaggtcaccgtctcctca

SEQ ID NO: 119 - DNA, 381 bp, Artificial Sequence
  NbRBD_H11-C6
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacgggttatacgtcttggctccttagcgactatgccacttggccttatgactactggggccaggggacccaggtcaccgtctcctca

SEQ ID NO: 120 - DNA, 381 bp, Artificial Sequence
  NbRBD_H11-E3
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagtgtatcatgctactcggtccctccttagcgactatgccacttggccttatgactactggggccaggggacccaggtcaccgtctcctca

SEQ ID NO: 121 - DNA, 381 bp, Artificial Sequence
  NbRBD_H11-F4
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagagagtacgattactcggtccctccttagcgactatgccacttggccttatgactactggggccaggggacccaggtcaccgtctcctca

SEQ ID NO: 122 - DNA, 381 bp, Artificial Sequence
  NbRBD_H11-C5
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacgtcgtatgtgagttttctccttagcgactatgccacttggccttatgactactggggccaggggacccaggtcaccgtctcctca

SEQ ID NO: 123 - DNA, 381 bp, Artificial Sequence
  NbRBD_H11-C2
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaggtagtcgggcgacgcggtccctccttagcgactatgccacttggccttatgactactggggccaggggacccaggtcaccgtctcctca

SEQ ID NO: 124 - DNA, 381 bp, Artificial Sequence
  NbRBD_H11-B11
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagagacgttgaatactcggtccctccttagcgactatgccacttggccttatgactactggggccaggggacccaggtcaccgtctcctca

SEQ ID NO: 125 - DNA, 381 bp, Artificial Sequence
  NbRBD_H11-A3
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacgttctgataatgtgcggtccctccttagcgactatgccacttggccttatgactactggggccaggggacccaggtcaccgtctcctca

SEQ ID NO: 126 - DNA, 381 bp, Artificial Sequence
  NbRBD_H11-D12
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagattggggtgtgactcggtccctccttagcgactatgccacttggccttatgactactggggccaggggacccaggtcaccgtctcctca

SEQ ID NO: 127 - DNA, 381 bp, Artificial Sequence
  NbRBD_H11-D6
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaagttcttctgtgacgcggtccctccttagcgactatgccacttggccttatgactactggggccaggggacccaggtcaccgtctcctca

SEQ ID NO: 128 - DNA, 381 bp, Artificial Sequence
  NbRBD_H11-F8
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagatgcttcggcgacgcggtccctccttagcgactatgccacttggccttatgactactggggccaggggacccaggtcaccgtctcctca